(19) United States
(12) Patent Application Publication
(10) Pub. No.: US 2023/0149318 A1
(43) Pub. Date: May 18, 2023

(54) COMBINATIONS OF NANOPARTICLE-ENCAPSULATED CARGO ENTITIES AND METHODS FOR MAKING AND USING SAME

(71) Applicant: Nurevelation, LLC, Raleigh, NC (US)

(72) Inventors: Tolan Naomie Lucas, Raleigh, NC (US); Eric William Seidel, Raleigh, NC (US); Davide Deodato, Hesingue (FR); César Andrés Carrasco-Lopez, New Haven, CT (US)

(21) Appl. No.: 17/985,636

(22) Filed: Nov. 11, 2022

Related U.S. Application Data
(60) Provisional application No. 63/293,408, filed on Dec. 23, 2021.
(60) Provisional application No. 63/278,696, filed on Nov. 12, 2021.

(51) Int. Cl. A61K 9/51; A61K 31/05; A61K 9/00; A61K 31/401; A61K 31/549; A61K 31/165; A61K 31/4184; A61K 31/343; A61K 31/57; A61K 31/7048; A61K 31/09; A61K 36/28; A61K 36/9066; A61K 31/573; A61K 36/258; A23L 27/30; A23L 27/00; A23L 2/60

(52) U.S. Cl. CPC A61K 9/5123; A23L 2/60; A23L 27/33; A23L 27/72; A23L 27/84; A61K 9/0095; A61K 9/5192; A61K 31/05; A61K 31/09; A61K 31/57; A61K 31/165; A61K 31/343; A61K 31/401; A61K 31/549; A61K 31/573; A61K 31/4184; A61K 31/7048; A61K 36/28; A61K 36/258; A61K 36/9066; A23V 2002/00

(57) ABSTRACT

Combinations of nanoparticle-encapsulated cargo entities and methods of making and using same.

### CROSS-REFERENCE TO RELATED APPLICATIONS

This application claims priority to U.S. Provisional Application No. 63/293,408, filed Dec. 23, 2021, and U.S. Provisional Application No. 63/278,696, filed Nov. 12, 2021, both of which are incorporated by reference in their entirety.

### BACKGROUND

Demand is increasing for foods, beverages, supplements, pharmaceuticals, and other comestible products that contain various entities such as active pharmaceutical ingredients (APIs), nutraceuticals, cannabinoids, among other comestible ingredients. Of particular interest are water-based or alcohol-based products, such as beverages and water-based comestibles that include one or more cargo entities of interest, in addition to solid or semi-solid comestibles. Unfortunately, many desirable entities have limited solubility in water. Other entities may be difficult to process or be difficult to effectively combine with other ingredients. Although progress has been made in producing water-soluble or alcohol-soluble products, current practices have several significant drawbacks.

For example, because many comestible ingredients have undesirable flavors, it is useful to develop a method that masks any bitter aftertaste and other taste properties of the ingredients in foods, beverages, supplements, pharmaceutical products, and other comestibles. However, current methods for incorporating ingredients into such products fail to adequately address the problem of taste. Current methods for incorporating certain ingredients into comestible products also have drawbacks with respect to the manufacturing process. For example, known methods generally require high volumes of additives to increase ingredient solubility, which can dilute the concentration of the active ingredient in the product.

Similarly, some methods for producing a water-soluble or alcohol-soluble product require the use of organic solvents to solubilize the ingredient, including for example, hexane, ethanol, and isopropyl alcohol. Other methods require the use of oils to encapsulate the ingredient, such as a carrier oil suitable for forming an oil-in-water emulsion. Such substances can be undesirable in comestible products for a variety of reasons, including toxicity and alteration of taste. In addition, known methods can also alter the active ingredient itself, which can result in the formation of unknown or undesirable byproducts.

It is therefore desirable to develop alternatives to existing techniques for incorporating ingredients into comestible products, including foods, beverages, and pharmaceutical products. Particularly, a need exists for a process capable for producing a water-soluble and/or alcohol-soluble nanoparticle-encapsulated cargo entity that is efficient, scalable, and which results in a product with a suitable concentration of the ingredient. It is also desirable to develop a water-soluble or alcohol-soluble compositions that are edible, that mask the taste of the unsavory ingredients, and that can easily be introduced into downstream product formulations, including foods and beverages, for example as a ready-to-use powder. Combining multiple ingredients can present further problems, thus creating a need for effective methods for preparing complex combination nanoparticle-encapsulated cargo entities, for example that can be ready-to-use by a consumer or easily diluted from powder form with a liquid such as water or alcohol (or process into a food) by a manufacturer. These needs and others are met by the following methods and products.

### SUMMARY

In one aspect, this disclosure relates to a composition comprising a plurality of particles encapsulating a cargo entity; wherein the particles comprise a sugar, sugar alcohol, or sugar substitute carrier that encapsulates the cargo entity; wherein at least two particles comprise a different cargo entity; wherein at least a first particle does not comprise the same cargo entity as at least a second particle; wherein each cargo entity is independently selected from cannabinoids, nutraceuticals, active pharmaceutical ingredients (APIs), minerals, inorganic or organic salts or compounds, supplements, amino acids, peptides, polypeptides, plant extracts, ubiquinones, herbs, vitamins, fatty acids, and any combination thereof; and wherein the particles have an average particle size ranging from about 200 nm to about 2,000 nm. In a further aspect, the particles have an average particle size ranging from about 1 nm to about 1,000 nm.

Also disclosed is a method for making a composition comprising a plurality of nanoparticle-encapsulated cargo entities, the method comprising: (a) recovering a first nanoparticle from a first dispersion comprising: (i) a sugar, sugar alcohol, or sugar substitute carrier that upon recovery encapsulates a first cargo entity; (ii) water; and (iii) the first cargo entity; wherein the mass ratio of the first cargo entity to the sugar, sugar alcohol, or sugar substitute is from about 1:1,000 to about 1:3; wherein the first cargo entity is selected from cannabinoids, nutraceuticals, active pharmaceutical ingredients (APIs), minerals, inorganic or organic salts or compounds, supplements, amino acids, peptides, polypeptides, plant extracts, ubiquinones, herbs, vitamins, fatty acids, and any combination thereof; and (b) recovering a second nanoparticle from a second dispersion comprising: (i) a sugar, sugar alcohol, or sugar substitute carrier that upon recovery encapsulates a second cargo entity; (ii) water; and (iii) the second cargo entity; wherein the mass ratio of the second cargo entity to the sugar, sugar alcohol, or sugar substitute is from about 1:1,000 to about 1:3; wherein the second cargo entity is selected from cannabinoids, nutraceuticals, active pharmaceutical ingredients (APIs), minerals, inorganic or organic salts or compounds, supplements, amino acids, peptides, polypeptides, plant extracts, ubiquinones, herbs, vitamins, fatty acids, and any combination thereof; wherein the second cargo entity is different from the first cargo entity; and (c) combining the first and second nanoparticles to provide the composition comprising the plurality of nanoparticle-encasulated cargo entities.

Still other objects and advantages of the present disclosure will become readily apparent by those skilled in the art from the following detailed description, which is shown and described by reference to preferred aspects, simply by way of illustration of the best mode. As will be realized, the disclosure is capable of other and different aspects, and its several details are capable of modifications in various respects, without departing from the disclosure. Accordingly, the description is to be regarded as illustrative in nature and not as restrictive.

### BRIEF DESCRIPTION OF THE DRAWINGS

The accompanying drawings, which are incorporated in and constitute part of this specification and together with the description, serves to explain the principles of the disclosure.

FIG. 1 is a process flow diagram illustrating an exemplary aspect of making a disclosed nanoparticle-encapsulated cargo entity.

FIG. 2 is a Transmission Electron Microscopy (TEM) image of an exemplary nanoparticle-encapsulated CBD sample prepared according to the disclosed methods.

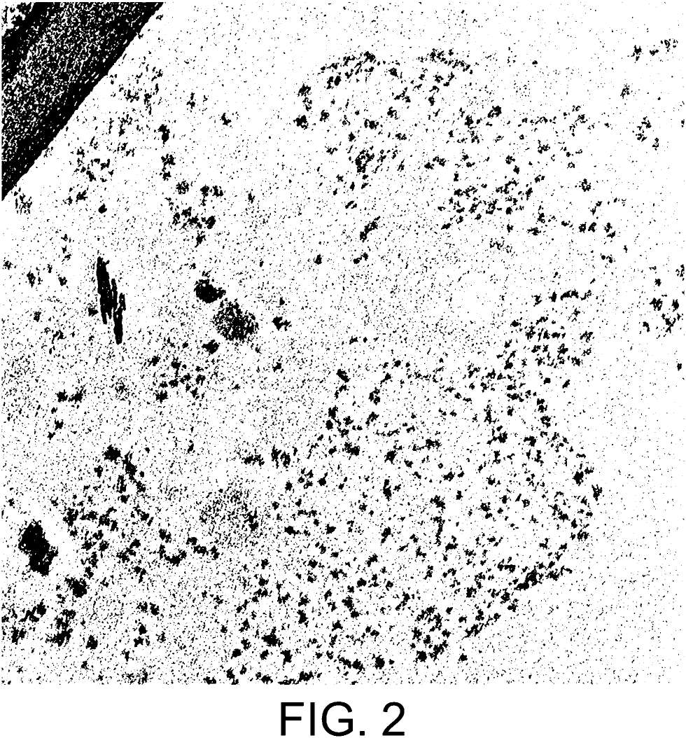

FIG. 3 shows plots of NMR spectra demonstrating the stability of CBD after encapsulation into nanoparticles using the disclosed methods. As shown, CBD remains intact after encapsulation (bottom spectrum) relative to a sample non-encapsulated CBD sample (top spectrum).

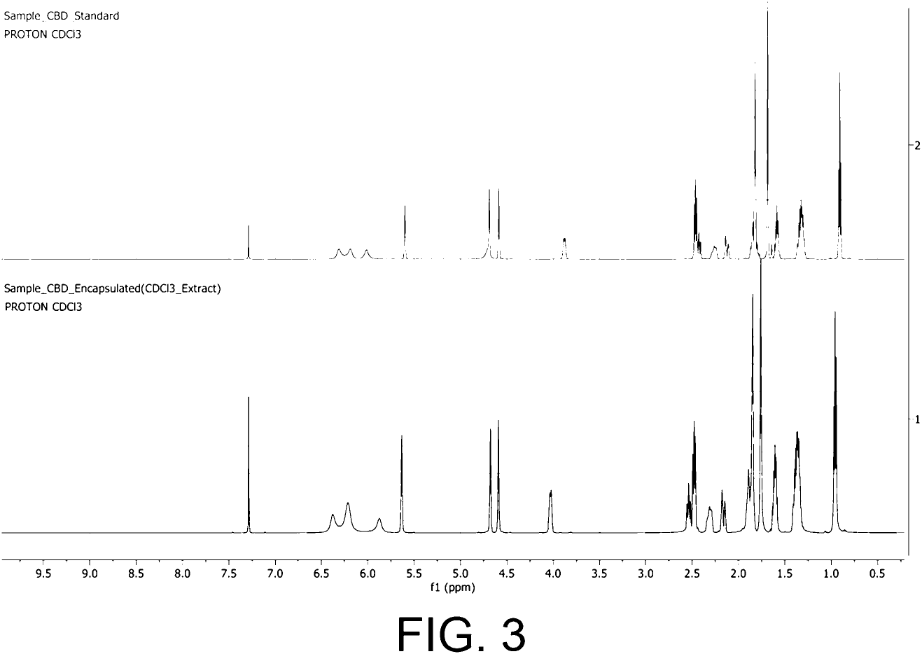

### DETAILED DESCRIPTION

The present invention can be understood more readily by reference to the following detailed description of the invention and the Examples included therein.

Disclosed are components that can be used to perform the disclosed methods. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and products. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

While aspects of this disclosure can be described and claimed in a particular statutory class, this is for convenience only and one of skill in the art will understand that each aspect of this disclosure can be described and claimed in any statutory class. Unless otherwise expressly stated, it is in no way intended that any method or aspect set forth herein be construed as requiring that its steps be performed in a specific order. Accordingly, where a method claim does not specifically state in the claims or description that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present application is not entitled to antedate such publication by virtue of prior invention. Further, stated publication dates may be different from actual publication dates, which can require independent confirmation.

### A. Definitions

Listed below are definitions of various terms. These definitions apply to the terms as they are used throughout this specification, unless otherwise limited in specific instances, either individually or as part of a larger group.

As used in the specification and in the claims, the term “comprising” can include the aspects “consisting of” and “consisting essentially of.”

As used in the specification and claims, the singular forms “a,” “an,” and “the” include plural referents unless the context clearly dictates otherwise.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

“Optional” or “optionally” means that the subsequently described event or circumstance may or may not occur, and that the description includes instances where said event or circumstance occurs and instances where it does not.

As used herein, the term “by weight,” when used in conjunction with a component, unless specially stated to the contrary is based on the total weight of the formulation or composition in which the component is included. For example, if a particular element or component in a composition or article is said to have 8% by weight, it is understood that this percentage is in relation to a total compositional percentage of 100%.

A weight percent of a component, or weight %, or wt%, unless specifically stated to the contrary, is based on the total weight of the formulation or composition in which the component is included.

References in the specification and concluding claims to parts by weight of a particular element or component in a composition or product, denote the weight relationship between the element or component and any other elements or components in the composition or product for which a part by weight is expressed. Thus, in a composition or a selected portion of a composition containing 2 parts by weight of component X and 5 parts by weight component Y, X and Y are present at a weight ratio of 2:5, and are present in such a ratio regardless of whether additional components are contained in the composition.

As used herein, the term “substantially,” in, for example, the context “substantially free of” refers to a composition having less than about 10% by weight, e.g., less than about 5%, less than about 1%, less than about 0.5%, less than about 0.1%, less than about 0.05%, or less than about 0.01% by weight of the stated material, based on the total weight of the composition.

It is further understood that the term “substantially,” when used in reference to a composition, refers to at least about 60% by weight, e.g., at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% by weight, based on the total weight of the composition, of a specified feature, component, or a combination of the components. It is further understood that if the composition comprises more than one component, the two or more components can be present in any ratio predetermined by one of ordinary skill in the art.

The terms “fruit water,” and “plant water,” as used herein, refer to the liquid fluid or juice that can be derived from the fruit, plant, or a vegetable produced by the plant. Non-limiting examples of fruit waters include coconut water, pineapple water, cherry water, mango water, apple water, pomegranate water, and the like. Non-limiting examples of plant or vegetable waters include cactus water, aloe vera water, beet water, carrot water, and the like.

As used herein, the term “sugar” is a collective term encompassing a variety of monosaccharides (e.g., glucose, dextrose, fructose, galactose), disaccharides (e.g., sucrose, lactose, maltose, trehalose), and oligosaccharides or polysaccharides (e.g., maltodextrin).

The term “sugar alcohol,” as used herein, refers to organic compounds, typically derived from a sugar, containing one hydroxyl group attached to each carbon atom. Sugar alcohols are also known as polyhydric alcohols, polyalcohols, alditols or glycitols.

As used herein, the term “sugar substitute” refers to a food additive that provides a sweet taste similar to that of sugar while containing less food energy than sugar-based sweeteners, e.g., a zero-calorie or low-calorie sugar substitute. Suitable sugar substitutes include those that do not include sucrose, fructose, or glucose, for example. The sugar substitute can be natural (e.g., plant derived) or artificial.

“Stevia,” as used herein, refers to any product derived from Stevia rebaudiana including the leaves thereof, and any product comprising steviol glycosides. Examples include without limitation liquid or solid stevia sweeteners, e.g., stevia extract, stevia leaf extract, stevia powder, stevia extract powder, organic stevia, sugar-free stevia, and the like.

The term “cannabinoid,” as used herein, refers to a class of chemical compounds capable of interacting with any mammalian cannabinoid receptor, for example the human CB₁ or CB₂ receptor. The term encompasses naturally-occurring cannabinoids (e.g., phytocannabinoids found in the cannabis plant), synthetic cannabinoids, cannabinoid mimetics, as well as salts, precursors, and metabolites thereof.

The term “nutraceutical” is used to describe medicinally or nutritionally functional food ingredients. Nutraceuticals have also been referred to as medical foods, designer foods, phytochemicals, functional foods and nutritional supplements, and include products such as vitamins, herbal remedies, genetically modified foods, and numerous supplements. In some instances, a nutraceutical may be a pharmaceutically active ingredient.

“Nanoparticle,” as used herein, refers to a solid nanoparticle entity formed by physical aggregation or noncovalent chemical association (e.g., through one or more noncovalent bonds) of two or more molecular entities, e.g., a cannabinoid and a sugar, sugar alcohol, or sugar substitute. In one aspect, a disclosed cannabinoid is encapsulated within the nanoparticle. “Nanoparticle-encapsulated cargo entity” as used herein, refers to a solid nanoparticle encapsulating one or more cargo entities with a sugar, sugar alcohol, or sugar substitute carrier, and includes for example, nanoparticles in which the cargo entity is at least partially encapsulated by the sugar, sugar alcohol, or sugar substitute. “Nanoparticle-encapsulated cargo entity” also includes nanoparticles in which the cargo entity and sugar, sugar alcohol, or sugar substitute is self-assembled through physical aggregation or noncovalent chemical association, in addition to nanoparticles that have a micelle or micelle-like structure. In one aspect,the disclosed nanoparticles have a size ranging from about 200 nm to about 2,000 nm, e.g., from about 200 nm to about 1,000 nm, or from about 200 nm to about 500 nm. In a further aspect, the disclosed nanoparticles have a size ranging from about 1 nm to about 1,000 nm, e.g., from about 1 nm to about 200 nm, or from about 1 nm to about 50 nm. Particle size can be determined using methods known in the art, e.g., transmission electron microscopy, light scattering, or zeta potential measurements. Particle size, as referred to herein, refers to the mean or average particle size of a given sample of a nanoparticle-encapsulated cargo entity.

“Fine powder” means a powder that can pass through a sieve with a nominal mesh aperture of 180 microns and not more than 40% by weight of the powder passes through a sieve with a nominal mesh aperture of 125 microns.

The term “mass ratio,” as used herein, refers to the mass of one substance (S1) relative to the mass of another substance (S2), where both masses have identical units (e.g., grams), expressed as S1:S2. For a substance such as water with a density of about 1 mg/mL, it is understood that reference to a volume of water (e.g., in mL) is equivalent to mass (e.g., in units of mg).

As used herein, the term “comestible” refers to any edible product or product suitable for ingestion by a human or animal. The term includes without limitation a liquid comestible (e.g., a water-based or alcohol-based product), and a solid or semi-solid comestible, including various food products, supplements, vitamins, ingestible therapeutic products, and the like.

As used herein, the term “subject” can be a vertebrate, a mammal, a fish, a bird, a reptile, an amphibian, or an invertebrate. Thus, the subject of the herein disclosed methods can be a human, non-human primate, horse, pig, rabbit, dog, sheep, goat, cow, cat, guinea pig or rodent. The subject can also be non-mammalian, e.g., a parakeet or a zebrafish. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subj ects.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). Thus, the term “subject” includes all categories of food-producing animals (e.g., livestock, poultry, and aquaculture species), companion animals (e.g., dogs, cats, horses, guinea pigs), laboratory animals, and animals maintained in zoological parks.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a clinical, medical, or physical examination by a person of skill, for example, a physician, and found to have a condition that can be diagnosed or treated by the compounds, compositions, or methods disclosed herein.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, “dosage form” means a pharmacologically active material in a medium, carrier, vehicle, or device suitable for administration to a subject. A dosage forms can comprise inventive a disclosed compound, a product of a disclosed method of making, or a salt, solvate, or polymorph thereof, in combination with a pharmaceutically acceptable excipient, such as a preservative, buffer, saline, or phosphate buffered saline. Dosage forms can be made using conventional pharmaceutical manufacturing and compounding techniques. Dosage forms can comprise inorganic or organic buffers (e.g., sodium or potassium salts of phosphate, carbonate, acetate, or citrate) and pH adjustment agents (e.g., hydrochloric acid, sodium or potassium hydroxide, salts of citrate or acetate, amino acids and their salts) antioxidants (e.g., ascorbic acid, alpha-tocopherol), surfactants (e.g., polysorbate 20, polysorbate 80, polyoxyethylene9-10 nonyl phenol, sodium desoxycholate), solution and/or cryo/lyo stabilizers (e.g., sucrose, lactose, mannitol, trehalose), osmotic adjustment agents (e.g., salts or sugars), antibacterial agents (e.g., benzoic acid, phenol, gentamicin), antifoaming agents (e.g., polydimethylsilozone), preservatives (e.g., thimerosal, 2-phenoxyethanol, EDTA), polymeric stabilizers and viscosity-adjustment agents (e.g., polyvinylpyrrolidone, poloxamer 488, carboxymethylcellulose) and co-solvents (e.g., glycerol, polyethylene glycol, ethanol). A dosage form formulated for injectable use can have a disclosed compound, a product of a disclosed method of making, or a salt, solvate, or polymorph thereof, suspended in sterile saline solution for injection together with a preservative.

The term “pharmaceutically acceptable” describes a material that is not biologically or otherwise undesirable, i.e., without causing an unacceptable level of undesirable biological effects or interacting in a deleterious manner.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

A temperature or temperature range, as expressed herein, refers to the temperature or temperature range at a pressure of 1 atm and equivalents thereof. For example, the phrase “at a temperature equivalent to from about 210° F. to about 280° F. at a pressure of 1 atm” refers not only to the temperature range at the stated atmospheric pressure but also to equivalent temperatures at lower and higher atmospheric pressures. Thus, a stated temperature range can encompass a lower equivalent temperature range at a pressure lower than 1 atm and a higher equivalent temperature range at a pressure higher than 1 atm. Similarly, in some aspects, a stated temperature range can encompass a higher equivalent temperature range at a pressure lower than 1 atm to achieve a kinetic energy equivalent to that achieved at the stated temperature range.

### B. Nanoparticle-Encapsulated Cargo Entities

In one aspect, this disclosure relates to a composition comprising a plurality of particles encapsulating a cargo entity; wherein the particles comprise a sugar, sugar alcohol, or sugar substitute carrier that encapsulates the cargo entity; wherein at least two particles comprise a different cargo entity; wherein each cargo entity is independently selected from cannabinoids, nutraceuticals, active pharmaceutical ingredients (APIs), minerals, inorganic or organic salts or compounds, supplements, amino acids, peptides, polypeptides, plant extracts, ubiquinones, herbs, vitamins, fatty acids, and any combination thereof; and wherein the particles have an average particle size ranging from about 200 nm to about 2,000 nm. In a further aspect, the particles have an average particle size ranging from about 1 nm to about 1,000 nm.

In one aspect, at least two particles comprise a different cargo entity. In one aspect, at least three particles comprise a different cargo entity. In one aspect, at least four particles comprise a different cargo entity. In one aspect, at least five particles comprise a different cargo entity. In one aspect, at least six particles comprise a different cargo entity. In one aspect, at least seven particles comprise a different cargo entity. In one aspect, at least eight particles comprise a different cargo entity. In one aspect, at least nine particles comprise a different cargo entity. In one aspect, at least ten particles comprise a different cargo entity. In one aspect, at least eleven particles comprise a different cargo entity. In one aspect, at least twelve particles comprise a different cargo entity. In one aspect, at least thirteen particles comprise a different cargo entity. In one aspect, at least fourteen particles comprise a different cargo entity. In one aspect, at least fifteen particles comprise a different cargo entity. In one aspect, at least sixteen particles comprise a different cargo entity. In one aspect, at least seventeen particles comprise a different cargo entity. In one aspect, at least eighteen particles comprise a different cargo entity. In one aspect, at least nineteen particles comprise a different cargo entity. In one aspect, at least 20 or more particles comprise a different cargo entity.

In a further aspect, at least a first particle in the composition comprises a cargo entity that is not the same as that encapsulated by at least a second particle. In other words, each cargo entity can be separately encapsulated according to the disclosed process, and individual batches of nanoparticle encapsulated cargo entities can be combined together to form a powdered composition, such that each nanoparticle comprises a different cargo entity that is not encapsulated by another nanoparticle in the composition. In one aspect, at least two nanoparticles comprise cargo entities that are not the same. In a further aspect, at least three nanoparticles comprise cargo entities that are not the same. In a further aspect, at least four nanoparticles comprise cargo entities that are not the same. In a further aspect, at least five nanoparticles comprise cargo entities that are not the same. In a further aspect, at least six nanoparticles comprise cargo entities that are not the same. In a further aspect, at least seven nanoparticles comprise cargo entities that are not the same. In a further aspect, at least eight nanoparticles comprise cargo entities that are not the same. In a further aspect, at least nine nanoparticles comprise cargo entities that are not the same. In a further aspect, at least ten nanoparticles comprise cargo entities that are not the same. In a further aspect, at least eleven nanoparticles comprise cargo entities that are not the same. In a further aspect, at least twelve nanoparticles comprise cargo entities that are not the same. In a further aspect, at least thirteen nanoparticles comprise cargo entities that are not the same. In a further aspect, at least fourteen nanoparticles comprise cargo entities that are not the same. In a further aspect, at least fifteen nanoparticles comprise cargo entities that are not the same. In a further aspect, at least sixteen nanoparticles comprise cargo entities that are not the same. In a further aspect, at least seventeen nanoparticles comprise cargo entities that are not the same. In a further aspect, at least eighteen nanoparticles comprise cargo entities that are not the same. In a further aspect, at least nineteen nanoparticles comprise cargo entities that are not the same. In a further aspect, at least 20 nanoparticles comprise cargo entities that are not the same.

In a further aspect, at least one particle in the composition can comprise two or more cargo entities. For example, certain B vitamins can solidify better when encapsulated together with other cargo entities. Vitamin B3 and Vitamin B5, for instance, can form a more solidified nanoparticle composition when co-encapsulated with Vitamin B1. Similarly, L-Arginine can form a more solidified nanoparticle composition when co-encapsulated with Curcumin. In one aspect, at least two particles in the composition can comprise two or more cargo entities, e.g., at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, or at least 20 particles in the composition can comprise two or more cargo entities.

In one aspect, the composition is any of the compositions listed below in Tables 2-57, without regard to the specific amounts of components listed. In one aspect, the composition is in the form of a fine powder. In one aspect, any of the compositions listed below in Table 2-57 can be formulated into a food, beverage, pharmaceutical, veterinary, or pet product. In one aspect, the composition can be diluted with 16 or 32 ounces of water and administered to a subject as described below. In one aspect, the composition can be diluted with an effective amount of alcoholic or non-alcoholic beverage and consumed. Specific non-limiting examples include beer, wine, cider, distilled spirit, hard seltzer, tea, coffee, milk, juice, fruit water, soft drinks and the like.

### C. Methods for Making the Nanoparticle-Encapsulated Cargo Entity

In one aspect, the disclosed cannabinoid nanoparticle can be made by recovering the nanoparticle from a dispersion comprising a sugar, sugar alcohol, or sugar substitute, water, and a cannabinoid, wherein the mass ratio of the cannabinoid to the sugar, sugar alcohol, or sugar substitute is from about 1:300 to about 1:3, and the dispersion comprises less than 10% by weight of alcohol.

### 1. Preparing the Dispersion

In various aspects, the dispersion can be prepared by mixing the desired amount of sugar, sugar alcohol, or sugar substitute, water, and cargo entity. In one aspect, the sugar, sugar alcohol, or sugar substitute can first be mixed with water, followed by addition of the desired amount of cargo entity. In one aspect, the water can be distilled, filtered, or otherwise purified to remove impurities typically present in tap water.

In one aspect, the water used for preparing the dispersion can be distilled, filtered, or otherwise purified and have a suitable pH. In a further aspect, the purified water used to prepare the dispersion can have a pH of from about 6 to about 9. In a still further aspect, the purified water used to prepare the dispersion can have a pH of from about 7 to about 8. In yet a further aspect, the purified water used to prepare the dispersion can have a pH within or near physiological limits, i.e., about 7 to about 8, or about 7.2 to about 7.5.

In some aspects, the water used for preparing the dispersion can be fruit or plant water, including water derived from a vegetable produced from a plant. Non-limiting examples of suitable fruit waters include coconut water, pineapple water, cherry water, mango water, apple water, pomegranate water, and the like. Non-limiting examples of suitable plant or vegetable waters include cactus water, aloe vera water, beet water, carrot water, and the like. In some aspects, the fruit or plant water can comprise from about 70% to about 98% water by weight. In a further aspect, the fruit or plant water comprising from about 70% to about 98% water by weight can be further diluted with additional water.

In one aspect, the dispersion can be prepared in a vessel comprising a surface that will not adhere to the cargo entity, e.g., stainless steel. The inventors have discovered that the use of vessels comprising Teflon and silicone results in certain cargo entities adhering to the Teflon or silicone. Similarly, in various aspects, any utensils used for stirring, mixing, or agitating the dispersion can be stainless steel or glass, as the inventors have discovered that the use of wooden utensils can result in certain cargo entities sticking to the wood.

In various aspects, the mass ratio of the sugar, sugar alcohol, or sugar substitute to the water can vary generally depending on the amount of cargo entity desired. In one aspect, for example, the mass ratio of the sugar, sugar alcohol, or sugar substitute to the water is from about 1:8 to about 1:1.2 prior to the recovering step, i.e., before the volume of the dispersion is reduced during any heating step. In a further aspect, the mass ratio of the sugar, sugar alcohol, or sugar substitute to the water is from about 1:8 to about 1:1.3 prior to the recovering step. In a still further aspect, the mass ratio of the sugar, sugar alcohol, or sugar substitute to the water is from about 1:8 to about 1:1.5 prior to the recovering step. In yet a further aspect, the mass ratio of the sugar, sugar alcohol, or sugar substitute to the water can be about 1:8, 1:7, 1:6, 1:5, 1:4, 1:3, 1:2, 1:1.5, 1:1.3, or 1:1.2 prior to recovering the nanoparticle-encapsulated cargo entity from the dispersion. In some aspects, the mass ratio of the sugar, sugar alcohol, or sugar substitute to the water is from about 1:6 to about 1:4 prior to the recovering step. In one aspect, the mass ratio of the sugar, sugar alcohol, or sugar substitute to the water is from about 1:4 prior to the recovering step.

In one aspect, for example, it is contemplated that when the amount of cargo entity added to the dispersion ranges from about 2-6 grams, the mass ratio of the sugar, sugar alcohol, or sugar substitute to the water can be about 3:4 (or about 1:1.33) or less. In a further aspect, when the amount of cargo entity added to the dispersion is 6 grams or above (e.g., 6-12 grams), the mass ratio of the sugar, sugar alcohol, or sugar substitute to the water can be at least about 1:4. In other words, according to some aspects, more water can be added to the dispersion as the amount of cargo entity added to the dispersion increases. Amounts of the cargo entity and other components of the dispersion can be scaled up as desired.

The mass ratio of the cargo entity to the sugar, sugar alcohol, or sugar substitute in the dispersion can vary. In one aspect, the mass ratio of the cargo entity to the sugar, sugar alcohol, or sugar substitute is from about 1:300 to about 1:3. For certain cargo entities, however, including vitamins such as Vitamin D3, it was discovered that the ratio of the entity to the sugar, sugar alcohol, or sugar substitute should about 1:1000. In another aspect, the mass ratio of the cannabinoid to the sugar, sugar alcohol, or sugar substitute is from about 1:50 to about 1:3. In a further aspect, the mass ratio of the cargo entity to the sugar, sugar alcohol, or sugar substitute is from about 1:50 to about 1:3. In another aspect, the mass ratio of the cargo entity to the sugar, sugar alcohol, or sugar substitute is from about 1:40 to about 1:3. In a further aspect, the mass ratio of the cargo entity to the sugar, sugar alcohol, or sugar substitute is from about 1:30 to about 1:10. In a still further aspect, the mass ratio of the cannabinoid to the sugar, sugar alcohol, or sugar substitute is from about 1:30 to about 1:15. For example, the mass ratio of the cannabinoid to the sugar, sugar alcohol, or sugar substitute can be about 1:50, 1:45, 1:40, 1:35, 1:30, 1:25, 1:20, 1:18, 1:16, 1:15, 1:13, 1:12, 1:11, 1:10, 1:9, 1:8, 1:7, 1:5, 1:4, or 1:3. In one aspect, the mass ratio of the cargo entity to the sugar, sugar alcohol, or sugar substitute is about 1:5.

### 2. Recovering the Nanoparticle-Encapsulated Cargo Entity

In one aspect, the dispersion comprising the sugar, sugar alcohol, or sugar substitute, water, and cargo entity can be heated at a temperature equivalent to from about 210° F. to about 280° F. at a pressure of 1 atm for a sufficient time to reduce the volume of the dispersion. As the volume of the dispersion decreases, the nanoparticle can form and solidify from the dispersion, e.g., by precipitation or coprecipitation of the cargo entity and the sugar, sugar alcohol, or sugar substitute, thereby forming the nanoparticle-encapsulated cargo entity. In a further aspect, the dispersion comprising the sugar, sugar alcohol, or sugar substitute, water, and cannabinoid can be heated at a temperature equivalent to from about 220° F. to about 240° F. at a pressure of 1 atm. In a still further aspect, the dispersion comprising the sugar, sugar alcohol, or sugar substitute, water, and cargo entity can be heated at a temperature equivalent to from about 225° F. to about 230° F. at a pressure of 1 atm. In yet another aspect, the dispersion comprising the sugar, sugar alcohol, or sugar substitute, water, and cargo entity can be heated at a temperature equivalent to a temperature at a pressure of 1 atm sufficient to induce the dispersion to boil for a sufficient time to reduce the volume of the dispersion and thereby solidify the nanoparticle-encapsulated cargo entity.

In some aspects, when the dispersion is heated to a temperature sufficient to induce the dispersion to boil for a sufficient amount of time at a stated pressure, equivalent boiling temperatures at different pressures can be calculated according to the Clausius-Clapeyron equation:

$$\text{ln} \left({P}_{2}\right) - ln \left({P}_{1}\right) = \frac{Δ {H}_{v a p}}{R} \left[\frac{1}{{T}_{1}} - \frac{1}{{T}_{2}}\right]$$

where P₁ and T₁ are standard atmospheric pressure and the known boiling point of water, respectively, ΔHᵥₐₚ is the enthalpy of vaporization of water, and R is the gas constant (8.3145 J/mol*K). Using the Clausius-Clapeyron equation, for example, it can be determined that the boiling temperature of water at 2 atm of pressure (about 247° F.) can be equivalent to the boiling temperature of water at 1 atm of pressure (about 212° F.). Other known methods for determining temperatures equivalent to stated temperatures at stated atmospheric pressures can also be used.

The dispersion can be heated to the desired temperature or temperature range using methods known in the art. In one aspect, for example, the dispersion can be heated in a suitable vessel (e.g., a stainless steel vessel) by a suitable heat source, such as, for example, an induction cooktop, or larger industrial equipment. The heat source can be maintained at a suitable temperature or temperature range such that the temperature of the dispersion stays at a temperature equivalent to from about 210° F. to about 280° F. at a pressure of 1 atm. Temperature of the dispersion can be monitored during the heating step using a thermometer, thermocouple, or other suitable device. The dispersion can also be heated under pressure at a suitable temperature equivalent to those described herein.

In various aspects, once the dispersion is heated for a sufficient time at the desired temperature, recovery of the nanoparticle-encapsulated cargo entity from the dispersion can include various steps. In some aspects, for example, the nanoparticle-encapsulated cargo entity can solidify or precipitate from the dispersion as the volume of the dispersion reduces to a certain level during heating. In one aspect, the dispersion can be heated at a temperature equivalent to from about 210° F. to about 280° F. at a pressure of 1 atm (e.g., equivalent to from about 220° F. to about 240° F. at 1 atm, or equivalent to from about 225° F. to about 230° F. at 1 atm) for a time sufficient to reduce the volume of the dispersion by about 10-95%, e.g., about 10%, 20%, 30%, 40%, 50%, 60%, 70%, 75%, 80%, 85%, 90%, or 95%, to thereby solidify or precipitate the nanoparticle-encapsulated cargo entity from the dispersion.

Optionally, as the volume of the dispersion comprising the sugar, sugar alcohol, or sugar substitute, water, and cargo entity decreases during the heating step, various additional steps can be performed to aid in the recovery of the nanoparticle-encapsulated cargo entity from the dispersion. In one aspect, for example, when the volume of the dispersion decreases to the desired level during the heating step, the dispersion can be agitated or stirred while maintaining the dispersion at the desired temperature or temperature range. In a further aspect, when the volume of the dispersion decreases to the desired level during the heating step, the dispersion can be agitated or stirred but not so vigorously as to create a vortex in the dispersion, while maintaining the dispersion at the desired temperature or temperature range. In one aspect, for example, the dispersion comprising the sugar, sugar alcohol, or sugar substitute, water, and cannabinoid can be agitated or stirred when the volume of the dispersion has decreased to the desired level, e.g., decreased by about 10-95%, e.g., about 10%, 20%, 30%, 40%, 50%, 60%, 70%, 75%, 80%, 85%, 90%, or 95%, to thereby solidify or precipitate the nanoparticle-encapsulated cargo entity from the dispersion.

In one aspect, as the volume of the dispersion comprising the sugar, sugar alcohol, or sugar substitute, water, and cargo entity decreases during the heating step, additional sugar, sugar alcohol, or sugar substitute can be added to the dispersion to aid in the solidification (e.g., by, optionally seeding the formation of a precipitate) of the nanoparticle-encapsulated cargo entity. For example, according to one aspect, additional sugar, sugar alcohol, or sugar substitute can be added in increments, e.g., increments of about 0.2 to about 0.5 grams, along with agitation or stirring, until visible solidification (e.g., precipitation) or agglomeration (e.g., syrup formation) of the nanoparticle-encapsulated cargo entity from the dispersion is observed. In a further aspect, solidification of the nanoparticle-encapsulated cargo entity from the dispersion can be aided by the use of an additive such as an antisolvent, e.g., a solvent that will not readily dissolve the nanoparticle-encapsulated cargo entity.

In some aspects, once solidification of the nanoparticle-encapsulated cargo entity from the dispersion has been observed, the dispersion can remain at the desired temperature equivalent to from about 210° F. to about 280° F. at 1 atm (e.g., equivalent to from about 220° F. to about 240° F. at 1 atm, or equivalent to from about 225° F. to about 230° F. at 1 atm) for a time sufficient to evaporate most or all of the liquid remaining in the dispersion. In another aspect, once solidification has been observed, the dispersion can be removed from the heat source with continuous agitation or stirring until the liquid remaining in the dispersion evaporates and the dispersion slowly cools. According to one aspect, for example, when the dispersion begins to attain the consistency of a slurry, the dispersion can be removed from the heat source and continuously agitated or stirred until the slurry resembles wet sand. Additional stirring or agitation can be performed until the slurry comprising the nanoparticle-encapsulated cargo entity dries to a solid, granular form, or in some instances, the nanoparticle-encapsulated cargo entity may remain a syrup which can be processed into a fine solidified powder if desired. Alternatively, according to other aspects, once solidification of the nanoparticle-encapsulated cargo entity occurs, e.g., when the dispersion begins to attain the consistency of a slurry, the dispersion can be filtered through a suitable filter to provide a filtride comprising the nanoparticle-encapsulated cargo entity. Similarly, in some aspects, once solidification of the nanoparticle-encapsulated cargo entity has occurred, the dispersion can be dried according to methods known in the art, e.g., drying under reduced pressure.

In a further aspect, the dispersion comprising the nanoparticle-encapsulated cargo entity can be cooled after the heating step (i.e., in which the dispersion is heated at a temperature equivalent to from about 210° F. to about 280° F. at 1 atm). In one aspect, for example, the dispersion can be heated at the desired temperature until there is no visible liquid (i.e., until the dispersion attains the consistency of a slurry), or until solid nanoparticle-encapsulated cargo entity formation begins to occur. The dispersion can then be removed from the heat and placed on a cooling tray with a larger surface area to facilitate uniform cooling (e.g., a stainless steel surface). This can in some aspects reduce the potential for the nanoparticle-encapsulated cargo entity to adhere to the heating vessel, which can result in lower yields. The cooling surface comprising the dispersion can be kept at a temperature generally less than 180° F. until the product appears dry and granular. In some aspects, the cooling surface can then be kept at a temperature of about 160° F. or below for a few minutes or a sufficient time to enable to nanoparticle product to solidify and cool.

Optionally, depending on the desired application, the dried nanoparticle-encapsulated cargo entity obtained from the dispersion can be ground into a powder. A powder of the nanoparticle-encapsulated cargo entity can be formed using methods known in the art, such as for example through the use of a food processor. In some aspects, once the nanoparticle-encapsulated cargo entity has been recovered from the dispersion, the solid nanoparticle-encapsulated cargo entity can be stored in a cool, dark, and dry environment until further use. In one aspect, for example, the nanoparticle-encapsulated cargo entity can be stored at a temperature equivalent to about 75° F. or less at a pressure of 1 atm (i.e., room temperature or below).

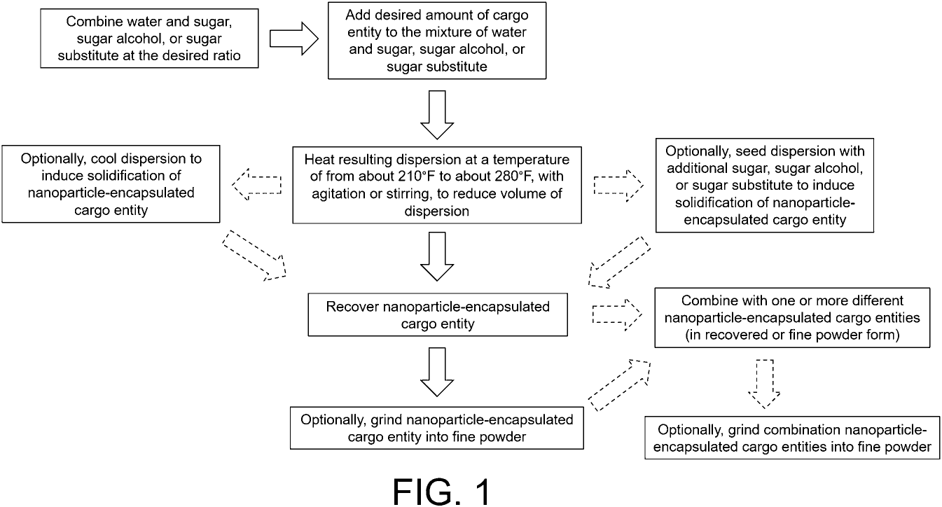

Referring now to FIG. 1, in one aspect, the method for making the nanoparticle-encapsulated cargo entity can comprise combining water and the sugar, sugar alcohol, or sugar substitute at the desired ratio, followed by adding the desired amount of cargo entity to the mixture of water and the sugar, sugar alcohol, or sugar substitute. The resulting dispersion can be heated at a temperature equivalent to from about 210° F. to about 280° F. at a pressure of 1 atm for a sufficient time to reduce the volume of the dispersion. As the volume of the dispersion decreases, the nanoparticle-encapsulated cargo entity can solidify from the dispersion (or form a syrup which can be subsequently solidified), depending on the dispersion composition. If the nanoparticle-encapsulated cargo entity does not readily solidify, optional steps can be carried out in which the dispersion is optionally seeded with additional sugar, sugar alcohol, or sugar substitute. Additionally, in some aspects, the dispersion can be cooled to induce solidification of the nanoparticle-encapsulated cargo entity. Once solidification of the nanoparticle-encapsulated cargo entity has occurred, the nanoparticle-encapsulated cargo entity can be recovered from the dispersion and optionally ground into a fine powder suitable for use in a consumer product such as those described below.

In addition, the nanoparticle-encapsulated cargo entity can be combined with another nanoparticle-encapsulated cargo entity (comprising a different cargo entity) either after the recovery step or after the optional grinding step. The combination of nanoparticle-encapsulated cargo entities can be ground into a fine powder (if not already) and provided as a dry powder suitable for formulation with a comestible such as a food, beverage, water, pharmaceutical product, and the like as further described below. In a further aspect, a beverage or other food or comestible product can be prepared by adding two or more separate cargo-containing nanoparticle compositions to the beverage or food product (i.e., adding one nanoparticle encapsulated cargo entity at a time for example as a pre-powder form).

### 3. Composition of the Dispersion

In one aspect, the nanoparticle-encapsulated cargo entity can be water- and/or alcohol-soluble and thus suitable for infusion into a water-based or alcohol-based comestible, or can be incorporated into any solid or semi-solid food or pharmaceutical product, or product for a pet such as a dog. Surprisingly, in one aspect, the inventors discovered that formation of the water-and/or alcohol-soluble nanoparticle-encapsulated cargo entity can be obtained from a dispersion that is substantially free of any solvent other than water, e.g., a dispersion comprising less than about 10% by weight of any non-water-based solvent such as an organic solvent (e.g., hexane, alcohols, and the like). For example, according to one aspect, the dispersion can be substantially free of alcohol, e.g., comprise less than about 10% by weight of alcohol, relative to the total weight of the dispersion. Thus, in some aspects, the nanoparticle-encapsulated cargo entity can be recovered from the dispersion without the use of an alcohol such as isopropyl alcohol or ethanol.

In a further aspect, the dispersion comprising the sugar, sugar alcohol, or sugar substitute, water, and cargo entity can be substantially free of ethanol. In one aspect, the dispersion can comprise less than about 10%, less than about 5%, less than about 1%, less than about 0.5%, less than about 0.1%, less than about 0.05%, or less than about 0.01% of ethanol.

In other aspects, the dispersion comprising the sugar, sugar alcohol, or sugar substitute, water, and cargo entity can be substantially free of isopropyl alcohol. In one aspect, for example, the dispersion can comprise less than about 10%, less than about 5%, less than about 1%, less than about 0.5%, less than about 0.1%, less than about 0.05%, or less than about 0.01% of isopropyl alcohol.

In a further aspect, the dispersion comprising the sugar, sugar alcohol, or sugar substitute, water, and cargo entity can be substantially free of ethanol and isopropyl alcohol, e.g., comprise less than about 10%, less than about 5%, less than about 1%, less than about 0.5%, less than about 0.1%, less than about 0.05%, or less than about 0.01% of ethanol and isopropyl alcohol.

In another aspect, the dispersion can be free of ethanol. In a further aspect, the dispersion can be free of isopropyl alcohol. In a still further aspect, the dispersion can be free of ethanol and isopropyl alcohol. Similarly, in some aspects, the dispersion can be free of any organic solvent, including for example hexane or alcohol-based solvents.

In one aspect, the nanoparticle-encapsulated cargo entity can be recovered from the dispersion comprising the sugar, sugar alcohol, or sugar substitute, water, and cargo entity without the use of a carrier oil to improve the solubility of the cargo entity, e.g., without creating an emulsion or other multi-phase system in the dispersion. Thus, in some aspects, the dispersion comprising the sugar, sugar alcohol, or sugar substitute, water, and cargo entity is free of any carrier oil, including but not limited to medium chain triglyceride (MCT) oil, long chain triglyceride (LCT) oil, vegetable oil, canola oil, olive oil, sunflower oil, coconut oil (including fractionated coconut oil), hemp oil, palm oils, and/or other oils suitable for human or animal consumption. Similarly, the dispersion comprising the sugar, sugar alcohol, or sugar substitute, water, and cargo entity can be free of other water-soluble agents used for creating an emulsion in the dispersion or improving the solubility of the cargo entity, including without limitation a starch such as a modified food starch, gum arabic, quillaja extract, or cyclodextrin. In some aspects, the dispersion can be similarly free of a sugar alcohol or maltodextrin, as further described below.

In a further aspect, the dispersion from which the nanoparticle-encapsulated cargo entity can be recovered consists essentially of the sugar, sugar alcohol, or sugar substitute, the water, and the cargo entity. In one aspect, as discussed below, the dispersion is free of a sugar alcohol, and thus the dispersion can consist essentially of the sugar or sugar substitute, the water, and the cannabinoid. Similarly, in some aspects, the dispersion is free of a sugar alcohol and sugar substitute and consists essentially of the sugar, the water, and the cargo entity.

In a still further aspect, the dispersion from which the nanoparticle-encapsulated cargo entity can be recovered consists of the sugar, sugar alcohol, or sugar substitute, the water, and the cannabinoid. In one aspect, as discussed below, the dispersion is free of a sugar alcohol, and thus the dispersion can consist of the sugar or sugar substitute, the water, and the cargo entity. Similarly, in some aspects, the dispersion is free of a sugar alcohol and sugar substitute and consists of the sugar, the water, and the cargo entity.

### A. Sugars, Sugar Alcohols, and Sugar Substitutes

A variety of sugars, sugar alcohols, and sugar substitutes are contemplated for use in the dispersion. In one aspect, the sugar, when present in the dispersion, can comprise allulose, glucose, dextrose, fructose, galactose, sucrose, lactose, maltose, trehalose, maltodextrin, or a combination thereof. In a further aspect, the sugar, when present in the dispersion, can comprise sucrose, fructose, glucose, or a combination thereof. In a still further aspect, the sugar, when present in the dispersion, can comprise sucrose, fructose, and glucose. In one aspect, for example, the sugar can be a naturally-occurring sugar, such as cane sugar, which comprises sucrose, fructose, and glucose.

In one aspect, sugar alcohols suitable for use with the dispersion can include without limitation ethylene glycol, glycerol, erythritol, threitol, arabitol, xylitol, ribitol, mannitol, sorbitol, galactitol, fucitol, iditol, inositol, volemitol, isomalt, maltitol, lactitol, maltotriitol, maltotetraitol, polyglycitol, or a combination thereof. In a further aspect, the sugar alcohol, when present in the dispersion, can comprise erythritol, xylitol, or a combination thereof.

In another aspect, the dispersion is substantially free of a sugar alcohol. According to one aspect, for example, the dispersion can comprise less than about 10%, less than about 5%, less than about 1%, less than about 0.5%, less than about 0.1%, less than about 0.05%, or less than about 0.01% of any sugar alcohol. In one aspect, the dispersion can be substantially free of glycol, glycerol, erythritol, threitol, arabitol, xylitol, ribitol, mannitol, sorbitol, galactitol, fucitol, iditol, inositol, volemitol, isomalt, maltitol, lactitol, maltotriitol, maltotetraitol, and polyglycitol. In a further aspect, the dispersion can be substantially free of isomalt, mannitol, sorbitol, xylitol, lactitol, maltitol, and erythritol.

In one aspect, the dispersion is free of a sugar alcohol. Thus, for example, according to one aspect, the dispersion is free of glycol, glycerol, erythritol, threitol, arabitol, xylitol, ribitol, mannitol, sorbitol, galactitol, fucitol, iditol, inositol, volemitol, isomalt, maltitol, lactitol, maltotriitol, maltotetraitol, and polyglycitol. In a further aspect, the dispersion is free of isomalt, mannitol, sorbitol, xylitol, lactitol, maltitol, and erythritol.

A variety of sugar substitutes are also contemplated for use with the dispersion. Suitable plant-derived sugar substitutes include without limitation brazzein, curculin, erythritol (also known as a sugar alcohol), fructooligosaccharide, glycyrrhizin, glycerol (also known as a sugar alcohol), hydrogenated starch hydrolysates, inulin, isomalt (also known as a sugar alcohol), isomaltooligosaccharide, isomaltulose, lactitol (also known as a sugar alcohol), mogroside mix, mabinlin, maltitol (also known as a sugar alcohol), maltodextrin (also referred to in some instances as a sugar), mannitol (also known as a sugar alcohol), miraculin, monatin, monellin, osladin, pentadin, polydextrose, psicose, sorbitol (also known as a sugar alcohol), stevia, tagatose, thaumatin, xylitol (also known as a sugar alcohol), or a combination thereof.

Suitable artificial sugar substitutes contemplated for use with the dispersion include without limitation acesulfame potassium, advantame, alitame, aspartame, salts of aspartame-acesulfame, sodium cyclamate, dulcin, glucin, neohesperidin dihidryochalcone, neotame, P-4,000, saccharin, sucralose, or a combination thereof. In a further aspect, the dispersion can comprise an artificial sweetener comprising sucralose.

In another aspect, the dispersion is substantially free of any sugar substitute. According to one aspect, for example, the dispersion can comprise less than about 10%, less than about 5%, less than about 1%, less than about 0.5%, less than about 0.1%, less than about 0.05%, or less than about 0.01% of any sugar substitute. In one aspect, the dispersion can be substantially free of brazzein, curculin, erythritol (also known as a sugar alcohol), fructooligosaccharide, glycyrrhizin, glycerol (also known as a sugar alcohol), hydrogenated starch hydrolysates, inulin, isomalt (also known as a sugar alcohol), isomaltooligosaccharide, isomaltulose, lactitol (also known as a sugar alcohol), mogroside mix, mabinlin, maltitol (also known as a sugar alcohol), maltodextrin (also referred to in some instances as a sugar), mannitol (also known as a sugar alcohol), miraculin, monatin, monellin, osladin, pentadin, polydextrose, psicose, sorbitol (also known as a sugar alcohol), stevia, tagatose, thaumatin, and xylitol (also known as a sugar alcohol). In a further aspect, the dispersion can be substantially free of acesulfame potassium, advantame, alitame, aspartame, salts of aspartame-acesulfame, sodium cyclamate, dulcin, glucin, neohesperidin dihidryochalcone, neotame, P-4,000, saccharin, and sucralose.

In another aspect, the dispersion is free of any sugar substitute. In one aspect, for example, the dispersion can be free of brazzein, curculin, erythritol (also known as a sugar alcohol), fructooligosaccharide, glycyrrhizin, glycerol (also known as a sugar alcohol), hydrogenated starch hydrolysates, inulin, isomalt (also known as a sugar alcohol), isomaltooligosaccharide, isomaltulose, lactitol (also known as a sugar alcohol), mogroside mix, mabinlin, maltitol (also known as a sugar alcohol), maltodextrin (also referred to in some instances as a sugar), mannitol (also known as a sugar alcohol), miraculin, monatin, monellin, osladin, pentadin, polydextrose, psicose, sorbitol (also known as a sugar alcohol), stevia, tagatose, thaumatin, and xylitol (also known as a sugar alcohol). In a further aspect, the dispersion can be free of acesulfame potassium, advantame, alitame, aspartame, salts of aspartame-acesulfame, sodium cyclamate, dulcin, glucin, neohesperidin dihidryochalcone, neotame, P-4,000, saccharin, and sucralose.

### B. Cargo Entities

Any suitable cargo entity that can be consumed by a subject can be used, as well as different cargo entities combined together to form combined nanoparticle-encapsulated cargo entity formulations. Examples include cannabinoids, nutraceuticals, active pharmaceutical ingredients (APIs), minerals, inorganic or organic salts or compounds, supplements, amino acids, peptides, polypeptides, plant extracts, ubiquinones, herbs, vitamins, fatty acids, and any combination thereof. Specific non-limiting examples of cargo entities, particular in combination with one another, are listed below in Tables 3-57.

In addition to active cargo entities, the disclosed compositions can include other ingredients such as flavorants, food products (e.g., bacon or bacon flavoring), among other ingredients. In some aspects, flavorants can be the cargo entity encapsulated by the carrier, e.g., bacon grease can be a cargo entity. In one aspect, the composition is free of any surfactant, dispersant, binder, or other agent or filler commonly present in oral tablet or pill forms of therapeutic agents, nutraceuticals, vitamins, and the like. As described elsewhere, a disclosed powdered composition can be packaged or can be incorporated into any comestible food product or beverage. Specific non-limiting examples include protein bars, gelatines, and the like.

### I. Cannabinoids

Cannabis is a genus of flowering plants that includes at least three species, Cannabis sativa, Cannabis indica, and Cannabis ruderalis. Cannabis plants produce a family of terpeno-phenolic compounds called cannabinoids. More than 100 cannabinoids have been identified from crude cannabis. Most cannabinoids exist in two forms, as acids and in neutral (decarboxylated) forms. The acid form is designated by an “A” at the end of its acronym, e.g., TCHA. Cannabinoids are synthesized in the plant as acid forms, and while some decarboxylation does occur in the plant, it increases significantly post-harvest, and the kinetics of decarboxylation increase at high temperatures. Decarboxylation can be achieved by thorough drying of the plant material followed by heating it or exposing it to light or alkaline conditions.

In various aspects, the disclosed dispersions include one or more cannabinoids. The cannabinoids can be in the acid or neutral form and can be derived from a cannabis plant or produced synthetically. Thus, in various aspects, the cannabinoid can be a cannabinoid acid. In some aspects, the cannabinoid in the dispersion can be Δ⁹-tetrahydrocannabinol (Δ⁹-THC), Δ⁸-tetrahydrocannabinol (Δ⁸-THC), Δ⁸-tetrahydrocannabiphorol (Δ⁸-THCP), Δ⁹-tetrahydrocannabiphorol (Δ⁹-THCP), cannabichromene (CBC), cannabicyclol (CBL), cannabidiol (CBD), cannabidiphorol (CBDP), cannabielsoin (CBE), cannabigerol (CBG), cannabinidiol (CBND), cannabinol (CBN), cannabitriol (CBT), or a combination thereof. The cannabinoid in the dispersion can also be in acid form, e.g., Δ⁹-THCA, Δ⁸-THCA, CBCA, CBLA, CBDA, CBEA, CBGA, CBNDA, CBNA, CBTA, or a combination thereof. In some aspects, the cannabinoid is CBD, CDPB, CBDA, THC, THCP, THCA, or a combination thereof. In further aspects, the cannabinoid is CBD, THC, or a combination thereof. In further aspects, the cannabinoid is CBD. In still further aspects, the cannabinoid is THC.

In other aspects, the dispersion is substantially free of THC (including Δ⁹-tetrahydrocannabinol (Δ⁹-THC), Δ⁸-tetrahydrocannabinol (Δ⁸-THC), Δ⁸-tetrahydrocannabiphorol (Δ⁸-THCP), and Δ⁹-tetrahydrocannabiphorol (Δ⁹-THCP)). In some aspects, the dispersion has less than about 10 wt% THC, less than about 5 wt% THC, less than about 3 wt% THC, less than about 1 wt% THC, less than about 0.8 wt% THC, less than about 0.7 wt% THC, less than about 0.6 wt% THC, less than about 0.5 wt% THC, less than about 0.4 wt% THC, less than about 0.3 wt% THC, less than about 0.2 wt% THC, less than about 0.1 wt% THC, less than about 0.05 wt% THC, or less than about 0.01 wt% THC, based on the total weight of the dispersion. In a further aspect, the dispersion is free of THC.

In some aspects, the dispersion is substantially free of CBN. In some aspects, the dispersion has less than about 10 wt% CBN, less than about 5 wt% CBN, less than about 3 wt% CBN, less than about 1 wt% CBN, less than about 0.8 wt% CBN, less than about 0.7 wt% CBN, less than about 0.6 wt% CBN, less than about 0.5 wt% CBN, less than about 0.4 wt% CBN, less than about 0.3 wt% CBN, less than about 0.2 wt% CBN, less than about 0.1 wt% CBN, less than about 0.05 wt% CBN, or less than about 0.01 wt% CBN, based on the total weight of the dispersion. In a further aspect, the dispersion is free of CBN.

In further aspects, the dispersion is substantially free of THC and CBN. Thus, in various further aspects, the dispersion has less than about 10 wt%, less than about 5 wt%, less than about 3 wt%, less than about 1 wt%, less than about 0.8 wt%, less than about 0.7 wt%, less than about 0.6 wt%, less than about 0.5 wt%, less than about 0.4 wt%, less than about 0.3 wt%, less than about 0.2 wt%, less than about 0.1 wt%, less than about 0.05 wt%, or less than about 0.01 wt%, of both THC and CBN, based on the total weight of the dispersion. In a further aspect, the dispersion is free of THC and CBN.

In some aspects, the cannabinoid in the dispersion can have a purity of at least 90%. In a further aspect, the purity of the cannabinoid in the dispersion ranges from 90-100%. In a still further aspect, the purity of the cannabinoid in the dispersion ranges from 92-97%. In general, the inventors have discovered that the encapsulation process described herein can be improved with the use of a pure cannabinoid starting material (e.g., from 92-97% pure). Similarly, the nanoparticle encapsulated cannabinoid products can advantageously maintain the purity of the cannabinoid over extended periods of time. In some aspects, the purity of the cannabinoid starting material can be maintained once encapsulated for a period of twelve months or longer, e.g., 18 months or longer, or even 24 months or longer. The stable shelf life of the cannabinoid nanoparticles is an advantage over many existing methods for delivering cannabinoid products. In some aspects, the stability of the cannabinoid can be measured using NMR, e.g., by comparing a pre-encapsulated cannabinoid to a cannabinoid extracted from a nanoparticle prepared by a disclosed method.

In various aspects, the one or more cannabinoids in the dispersion can be obtained commercially, prepared synthetically, or extracted from a cannabis plant. In other aspects, synthetic biology methods can be used to prepare the cannabinoids, e.g., through microbial factories. Once harvested, cannabis plant material typically includes flowers, leaves, and/or stems. In some aspects, cannabis plant material can be frozen for a suitable period of time, e.g., 36 hours, prior to being dried and extracted. Once dried, cannabis plant material can be extracted using a variety of techniques, including hydrocarbon extraction and supercritical CO₂ extraction.

In some aspects, the dispersion can comprise neutral cannabinoids, which can be prepared by decarboxylating cannabinoid acids. In various aspects, cannabinoid acids obtained from cannabis plant material can be decarboxylated by heating the dried plant material at a temperature of about 220° F. for at least 10-15 minutes followed by heating for about 280° F. for at least 45 minutes. Other known methods for decarboxylating cannabinoid acids from cannabis plant material can also be used.

According to one aspect, the cannabinoid in the dispersion can be provided from a cannabinoid composition that comprises a certain amount of cannabinoid. In some aspects, for example, the cannabinoid can be provided from a composition that comprises at least about 90% by weight of a cannabinoid. In a further aspect, the cannabinoid can be provided from a composition that comprises at least about 95% by weight of a cannabinoid. In a still further aspect, the cannabinoid can be provided from a composition that comprises at least about 99% by weight of a cannabinoid. For example, in one aspect, the cannabinoid is provided from commercially-available CBD isolate, which is typically a crystalline or solid powder comprising 99% CBD by weight.

In a further aspect, one or more cannabinoids can be in the dispersion and final cannabinoid nanoparticle. Thus, for example, the dispersion and final cannabinoid nanoparticle can comprise two or more, three or more, or four or more cannabinoids. In one aspect, the dispersion and final cannabinoid nanoparticle can comprise two different cannabinoids

### II. APIs and Nutraceuticals

In various aspects, the disclosed dispersions and resulting compositions include one or more therapeutic agents or nutraceuticals, for example in combination with other nanoparticle-encapsulated cargo entities. The therapeutic agent or nutraceutical can be naturally occurring, e.g., derived from a cell or plant, or can be produced synthetically. In a further aspect, the therapeutic agent or nutraceutical can be produced by genetically modified cells or microbial factories.

In some aspects, when the therapeutic agent is a solid at a temperature equivalent to 25° C. at a pressure of 1 atm, the therapeutic agent has a melting point of 70° C. to 340° C. In a further aspect, when the therapeutic agent is a solid at a temperature equivalent to 25° C. at a pressure of 1 atm, the therapeutic agent has a melting point of 100° C. to 340° C. In additional aspects, when the therapeutic agent is a liquid at a temperature equivalent to 25° C. at a pressure of 1 atm, the therapeutic agent has a boiling point of at least 130° C., e.g., 130° C. to 200° C.

In one aspect, the therapeutic agent or nutraceutical can be stable, i.e., will not degrade, at the temperatures used in the disclosed method. Thus, in some aspects, the therapeutic agent or nutraceutical can be stable for at least about 30 minutes when exposed to a temperature of from about 90° C. to about 122° C. at a pressure of 1 atm. In a further aspect, the therapeutic agent or nutraceutical can be stable for at least about 30 minutes when exposed to a temperature of from about 104° C. to about 116° C. at a pressure of 1 atm. In a still further aspect, the therapeutic agent or nutraceutical can be stable for at least about 30 minutes when exposed to a temperature of from about 107° C. to about 110° C. at a pressure of 1 atm.

The therapeutic agent or nutraceutical can have a variety of molecular weights. It is contemplated that both small molecules and larger molecules, e.g., biologics, peptides, amino acid chains, and the like, can be incorporated into the dispersion. For larger molecules such as collagen for example, the disclosed process results in complexation of the carrier and the collagen. Collagen and other large molecules with molecular weight up to and exceeding 250,000 daltons (Da) are contempleted for use with the disclosed methods and compositions. According to a further aspect, the therapeutic agent or nutraceutical has a molecular weight of about 6,000 daltons (Da) or less. In a further aspect, the therapeutic agent or nutraceutical has a molecular weight of about 1,000 daltons (Da) or less. In a still further aspect, the therapeutic agent or nutraceutical has a molecular weight of from about 150 g/mol to about 1,000 g/mol. In a still further aspect, the therapeutic agent or nutraceutical has a molecular weight of from about 150 g/mol to about 500 g/mol. In a further aspect, the therapeutic agent, when present, has a molecular weight of 120 to 1,000 g/mol. The units “g/mol” are equivalent to daltons (Da). In a further aspect, the therapeutic agent comprises at least one aromatic group, including for example, an aryl or heteroaryl group as defined above.

According to one aspect, the therapeutic agent or nutraceutical has an octanol-water partition coefficient (logP) of from about -5 to about 15. In a further aspect, the therapeutic agent or nutraceutical has an octanol-water partition coefficient (logP) of from about -2 to about 10. In a further aspect, the therapeutic agent or nutraceutical has a pKa of from about 1 to about 13, e.g., about 2, 3, 4, 5, 6, 7, 8, 9, 10, 11, 12, or 13. According to a further aspect, the therapeutic agent or nutraceutical has a water solubility of at least about 0.001 pg/mL.

The effective amount of the therapeutic agent or nutraceutical present in the dispersion can vary depending on the therapeutic agent or nutraceutical and its use. According to one aspect, the effective amount of the therapeutic agent or nutraceutical is a therapeutically effective amount. According to a further aspect, the effective amount of the therapeutic agent or nutraceutical is a prophylactically effective amount.

A wide variety of therapeutic agent or nutraceuticals can be incorporated into the disclosed dispersions to form a therapeutic agent or nutraceutical composition. According to one aspect, the therapeutic agent or nutraceutical is a medicament, vitamin, mineral supplement, or a substance used for the treatment, prevention, diagnosis, cure, or mitigation of a disease or illness.

In a further aspect, the therapeutic agent is a biologic, an adjuvant, anti-infective agent, anti-cancer agent, anti-neoplastic agent, poly ADP ribose polymerase (PARP) inhibitor, DNA damage response modifier, epigenetic agent, histone deacetylase (HDAc) inhibitor, iron chelator, ribonucleotide reductase inhibitor, proteasome inhibitor, Nedd8-activating enzyme (NAE) inhibitor, mammalian target of rapamycin (mTOR) inhibitor, cytotoxic agent, immune checkpoint blockade agent, toll-like receptor (TLR) agonist, immune modifier, cell therapeutic, anti-ALS agent, analgesic, anorexic, anti-inflammatory agent, anti-epileptic agent, anesthetic, hypnotic, sedative, antipsychotic agent, neuroleptic agent, antidepressant, anxiolytics, antagonist, neuron blocking agent, anticholinergic agent, cholinomimetic agent, antimuscarinic agent, muscarinic agent, antiadrenergic, antiarrhythmic, antihypertensive agent, hormone, nutrient, nutraceutical, antiarthritic, antiasthmatic, anticonvulsant, antihistamine, antinauseant, antineoplastic, antipruritic, antipyretic, antispasmodic, cardiovascular preparation, diuretic, vasodilator, central nervous system stimulant, cough or cold agent, decongestant, diagnostic, bone growth stimulant, bone resorption inhibitor, immunosuppressive, muscle relaxant, psychostimulant, tranquilizer, pain or fever reducer, antiviral, antiretroviral, antibacterial, platelet reducer, anticoagulant, an agent for treating muscle spasms, or an agent for treating dry mouth.

According to one aspect, two or more therapeutic agents or nutraceuticals can be present in the dispersion. Alternatively, according to a further aspect, a therapeutic agent or nutraceutical composition can be prepared with a first therapeutic agent or nutraceutical, and one or more additional therapeutic agent or nutraceutical complexes or syrups can be prepared with additional therapeutic agent(s) or nutraceutical(s), and the resulting complexes or syrups can be mixed together to form a composite composition. Such a composite composition may be useful for example in preventing unwanted interaction between two or more therapeutic agents or nutraceuticals.

In one aspect, for example, a single composition or a composite of such complexes or syrups can comprise one or more antibacterial therapeutic agents, e.g., β-lactam antibiotic plus an aminoglycoside, which can be useful for subjects with bacteremia and neutropenia. Similarly, for example, infections with Pseudomonas aeruginosa or Acinetobacter baumannii isolates that are resistant to antibiotics except polymyxins, several antibiotic combinations can demonstrate increased activity compared with that of any single agent.

In a further aspect, the following combination therapies are contemplated for use with the therapeutic agent complexes or syrups: Pylera (bismuth sub/metronidazole/tetracycline), TRUVADA (emtricitabine/tenofovir), Sinemet (carbidopa/levodopa), BIKTARVY (bictegruvir/emtricitabine/tenofivir), Lisinopril-hydrochlorothiazide, Amlodipine/atorvastatin, Hydrocodome-Apap, Oxycodone-Apap, Buprenorphine-naloxone, Atenolol-chlorthalidone, Xigduo (Dapagliflozin/Metformin), various prenatal vitamins (e.g., Folic acid) comprising multiple vitamins/minerals/DHA, among other combination therapies known in the art.

According to a further aspect, the therapeutic agent or nutraceutical is not a cannabinoid. In other aspects, the therapeutic agent or nutraceutical is not an agent known for treating or preventing cough. According to one aspect, for example, the therapeutic agent or nutraceutical does not include dextromethorphan, camphor, eucalyptus oil, menthol, guaifenesin, or any combination thereof. In a further aspect, the therapeutic agent or nutraceutical composition does not comprise caffeine.

In a further aspect, the therapeutic agent can be a non-steroidal anti-inflammatory, e.g., aspirin, a carboxylic acid-containing therapeutic agent, an acidic or basic amino acid, Imatinib, doxycycline for a variety of subjects including mammals, e.g., pets or human subjects, levofloxacin, ivermectin, or an anti-HIV drug.

In a still further aspect, the therapeutic agent can be any of the agents listed in Table 1A below together or in any logical or therapeutically useful combination. Any of the agents below can be present either in neutral or salt form, including without limitation any of the salt forms approved by the FDA. Thus, although certain salts and neutral counterparts are specifically described and listed, it is contemplated that when a neutral form is described, salts or the neutral form are described, and when a salt form is described, neutral counterparts of the salt are described.

Exemplary Therapeutic Agents

| Compound | Use | Molecular Weight (g/mol) |
|---|---|---|
| Acetaminophen | Pain/Fever Reduction | 151.163 |
| Acyclovir | Antiviral | 225.21 |
| Albuterol sulfate | Asthma | 576.7 |
| Amiodarone hydrochloride | Heart Rhythm | 645.31 |
| Amoxicillin | Anti-bacterial | 365.4 |
| Amphetamine aspartate | ADD | 421.5 |
| Anagrelide hydrochloride | Platelet Reducer | 292.5 |
| Anastrozole | Breast Cancer | 293.374 |
| Apixaban | Anticoagulant | 459.5 |
| Aripiprazole | Atypical antipsychotic | 448.4 |
| Aspirin | Pain | 180.16 |
| Atenolol | Beta blocker | 266.34 |
| Baclofen | muscle spasms | 213.67 |
| Benztropine | Used to treat a type of movement disorder due to antipsychotics known as dystonia and parkinsonism. Benefits are seen within 2 hours and last for up to 10 hours | 307.44 |
| Bethanechol chloride (bethanechol) | dry mouth | 196.68 |
| Bisoprolol fumarate | High blood pressure | 325.44 |
| Bisacodyl | Laxative | 361.39 |
| Brompheniramine maleate | antihi stamine | 435.3 |
| Budesonide | Crohn’s disease and ulcerative colitis | 430.53 |
| Buspirone | Anxiety | 385.51 |
| Buspirone hydrochloride | Anxiety | 422 |
| Butalbital | Pain | 224.26 |
| Carbamazepine | seizures, nerve pain, and bipolar disorder | 236.27 |
| Carvedilol | Heart failure, left ventricular dysfunction, and hypertension. Belongs to a class of drugs known as alpha and beta blockers. | 713.92 |
| Cedazuridine | myelodysplastic syndromes (MDS) | 268.21 |
| Cetirizine | Second-generation histamine H1 antagonist | 388.89 |
| Chlordiazepoxide Hydrochloride | anxiety, alcohol withdrawal symptoms, and tremor | 336.2 |
| Chlorzoxazone | pain and stiffness caused by muscle spasm | 169.57 |
| Chloroquine | Immunosuppressive and anti-parasite | 319.88 |
| Citalopram | Antidepressant | 324.4 |
| Clemastine fumarate | hay fever and allergy symptoms | 460 |
| Clidinium bromide | peptic ulcer | 432.3 |
| Clopidogrel | Antiplatelet | 321.82 |
| Colchicine | Anti-inflammatory | 399.44 |
| Colesevelam hydrochloride | cholesterol levels and treat type 2 diabetes. | 618.2 |
| Cyclobenzaprine hydrochloride | pain and discomfort related to muscle spasms, sprains and other muscle injuries | 311.8 |
| Cysteamine bitartrate | Cystine reducer | 227.24 |
| Decitabine | myelodysplastic syndromes (MDS) | 228.21 |
| Deferasirox | chronic iron overload | 373.4 |
| Dextromethorphan, hydrobromide | Cough relief | 370.3 |
| Diazoxide | low blood sugar (hypoglycemia) | 230.67 |
| Diphenylhydantoin | Anticonvulsant | 252.27 |
| Donepezil | Cognition-enhancing | 379.49 |
| Doxepin Hydrochloride | depression, anxiety, and sleep disorders | 315.8 |
| Doxycycline | Anti-bacterial | 444.4 |
| Doxycycline hyclate | Anti-bacterial | 512.9 |
| Doxylamine | hay fever and other allergy symptoms | 270.37 |
| Doxylamine Succinate | hay fever and other allergy symptoms | 388.5 |
| Dutasteride | Urinary retention | 528.5 |
| D-penicillamine | Immunosuppressive drug and Heavy metal toxicity medication | 149.21 |
| Eletriptan hydrobromide | migraine headaches | 463.4 |
| Erlotinib hydrochloride | cancer, including pancreatic and non-small cell lung cancer | 429.9 |
| Erythromycin | Antibiotics and Gut motility stimulator | 733.93 |
| Estazolam | insomnia | 294.7 |
| Ethinyl estradiol | estrogen medication | 296.4 |
| Everolimus | cancer of the kidney, pancreas, breast, and brain | 958.2 |
| Famotidine | Pepetic ulcer disease, gastroesophageal reflux disease (GERD), Zollinger-Ellison syndrome | 337.44 |
| Fenofibrate | Cholesterol | 360.83 |
| Fingolimod | multiple sclerosis (MS) | 343.9 |
| Fluconazole | fungal infections | 306.27 |
| Fluphenazine | schizophrenia | 437.52 |
| Fluphenazine hydrochloride | schizophrenia | 510.4 |
| Fluoxetine hydrochloride | Antidepressant, Separation Disorder in pets such as dogs | 345.79 |
| Fostemsavir tromethamine | antiretroviral medication (HIV) | 704.6 |
| Glimepiride | Anti-diabetic | 490.62 |
| Glipizide | Anti-diabetic | 445.54 |
| Griseofulvin | fungal infections | 352.76 |
| Guaifenesin | Expectorant (cough and cold) | 198.22 |
| Guanfacine Hydrochloride | antihypertensive | 282.5 |
| Hydrochlorothiazide | high blood pressure and fluid retention (edema) | 297.74 |
| Ibuprofen | Non-steroidal anti-inflammatory | 206.29 |
| Imatinib | Chronic myelogenous leukemia, leukemia, leukemia (CML), gastrointestinal stromal tumors (GISTs), and other malignancies | 493.62 |
| Imatinib Mesylate | leukemia and other kinds of cancer | 589.7 |
| Insulin | Blood sugar control | 5734 |
| Irbesartan | Antihypertensive | 428.5 |
| Itraconazole | Fungal infections in immunocompromised and non-immunocompromised patients: pulmonary and extrapulmonary blastomycosis, histoplasmosis, aspergillosis, and onychomycosis. | 705.64 |
| Lamotrigine | Mood stabilizer, bipolar disorder, seizures | 256.1 |
| Leucovorin calcium | prevent the side effects of a cancer medication | 473.44 |
| Levothyroxine | Thyroid hormone deficiency, including myxedema coma. Also used to treat and prevent certain types of thyroid tumors. | 776.87 |
| Liothyronine sodium | Hypothyroidism | 650.97 |
| Lidocaine | Antiarrhythmic and Anesthetic | 234.34 |
| Levodopa | Parkinson’s disease and dopamine-responsive dystonia | 197.19 |
| Levofloxacin | Antibiotic to treat bacterial infections including acute bacterial sinusitis, pneumonia, urinary tract infections, chronic prostatitis and some type of gastroenteritis. Along with other antibiotics, it may be used to treat tuberculosis, meningitis or pelvic inflammatory disease. | 361.37 |
| Lisinopril dihydrate | ACEI inhibitor, hypertension, heart failure, myocardial infarction | 441.53 |
| Loratadine | Allergies, including allergic rhinitis (hay fever) and hives | 382.89 |
| Lovastatin | High cholesterol and triglyceride | 404.54 |
| Meloxicam | Nonsteroidal anti-inflammatory drug | 351.4 |
| Memantine | Cognition-enhancing medication | 179.3 |
| Mesalamine | Nonsteroidal anti-inflammatory drug | 153.13 |
| Metaxalone | Muscle relaxant | 221.25 |
| Methotrexate sodium | cancer of the blood, bone, lung, breast, head, and neck | 454.44 |
| Methylphenidate | ADHD and narcolepsy | 233.31 |
| Metronidazole | Antibacterial, skin redness | 171.16 |
| Metyrosine | symptoms caused by adrenal gland tumors (pheochromocytoma) | 195.22 |
| Mexitil (Mexiletine HCl) | irregular heartbeats (arrhythmias) | 215.72 |
| Mifepristone | Progesterone blocker | 429.6 |
| Mycophenolate Mofetil | prevent organ rejection after transplant | 433.49 |
| Naloxegol oxalate | constipation caused by opiate (narcotic) pain medications in adults with chronic (ongoing) pain | 741.8 |
| Naproxen | Nonsteroidal anti-inflammatory | 230.26 |
| Naloxone | Opiate overdose | 327.3 |
| Nifedipine | Antihypertensive drug and Calcium channel blocker | 346.3 |
| Norgestimate | estrogen medication | 369.5 |
| Olanzapine | mental disorders, including schizophrenia and bipolar disorder | 312.4 |
| Olmesartan medoxomil | high blood pressure | 558.59 |
| Ondanestron | nausea and vomiting | 293.4 |
| Oxycodone hydrochloride | Pain | 315.36 |
| Penicillamine | Immunosuppressive drug and Heavy metal toxicity medication | 149.21 |
| Phenytoin | Anticonvulsant | 252.27 |
| Piaglitazone | Anti-diabetic | 356.44 |
| Pioglitazone hydrochloride | Anti-diabetic medication | 392.9 |
| Plavix (Clopidogrel Bisulfate) | prevent stroke, heart attack, and other heart problems | 419.9 |
| Prasugrel hydrochloride | Blood thinner | 409.9 |
| Pravachol (Pravastatin Sodium) | high cholesterol and triglyceride levels | 446.5 |
| Prednisone | Immunosuppressant, inflammation, etc. | 358.43 |
| Pregabalin | Nerve pain medication | 159.23 |
| Prucalopride succinate | impaired motility associated with chronic constipation | 486 |
| Pyrazinamide | tuberculosis | 123.11 |
| Pyridoxine Hydrochloride | dietary supplement | 205.64 |
| Ranitidine | Antihistamine and Antacid | 314.4 |
| Rosuvastatin | high cholesterol and triglyceride levels | 481.5 |
| Risperidone | Antipsychotic | 410.49 |
| Safinamide Mesylate | idiopathic Parkinson’s disease (PD) | 302.34 |
| Selegiline (BBB) | Parkinson’s and major depressive disorder | 187.29 |
| Sertraline | Antidepressant | 306.23 |
| Sildenafil | Vasodilator | 474.6 |
| Spironolactone | high blood pressure | 416.57 |
| Sulfamethoxazole | Antibiotic | 253.28 |
| Tadalafil | erectile dysfunction and enlarged prostate | 389.4 |
| Tolcapone | symptoms of Parkinson’s disease | 273.25 |
| Tolterodine tartrate | overactive bladder symptoms | 475.6 |
| Triheptanoin | long-chain fatty acid oxidation disorders | 428.6 |
| Trimethoprim | Antibiotics and Folate synthesis inhibitor | 290.32 |
| Ultramicrosize | antifungal | 352.8 |
| Vancomycin | Antibiotic | 1,449.30.00 |
| Vardenafil | Erectile dysfunction | 525.1 |
| Venlafaxine | Antidepressant and Nerve pain medication | 277.4 |
| Voriconazole | fungal infections | 349.31 |
| Warfarin | Blood thinner | 308.33 |
| Zidovudine | HIV antiviral | 267.24 |

In a further aspect, the therapeutic agent can be a non-steroidal anti-inflammatory, e.g., aspirin, a carboxylic acid-containing therapeutic agent, an acidic or basic amino acid, Imatinib, doxycycline for a variety of subjects including mammals, e.g., pets or human subjects, levofloxacin, ivermectin, or an anti-HIV drug.

In one aspect, the nutraceutical can be any of the agents listed in Table 1B below either alone or in combination.

Exemplary Nutraceuticals

| | |
|---|---|
| Nutraceutical | Reported Benefits |
| Moringa | Anti-oxidant and anti-inflammatory |
| Vitamin D3 | Various |
| L-Theanine | Anti-anxiety |
| Methylsulfonylmethane | Anti-inflammatory |
| (MSM) | |
| Asian Ginseng | Performance |
| Guarana | Anti-oxidant and stimulant |
| Ginger Root | Gastroprotective, Anti-oxidant/anti-inflammatory, |
| | Neuroprotective, Cardioprotective |
| Ceylon Cinnamon | Various |
| Curcumin | Anti-inflammatory |
| Magnesium Oxide | Performance |
| Tumeric | Anti-inflammatory |
| Caralluma Fimbrita | Various |
| Beet Root | Various |
| Green Tea Extract | Various |
| Garcinia Cambogia | Various |
| Conjugated linoleic acid (CLA) | Various |
| CoQ10 | Anti-oxidant/anti-inflammatory |
| Maca | Various |
| Echinacea | Various |
| L-Tryptophan | Anti-anxiety |
| Coconut Oil | Various |
| Milk Thistle | Anti-oxidant |
| Lime | Various |
| Valerian Root | Anti-anxiety |
| St. John’s Wort | Various |
| Kava Tincture | Various |
| Black seed oil | Various |
| Melatonin | Sleep aid |
| L-Carnitine | Various |
| L-Thyroxine | Various |
| Lemongrass | Various |
| Lemon | Various |
| Fennel | Various |
| Oregano | Various |
| Peppermint | Various |

In a further aspect, any of the following categories and examples therein can be cargo entities in the combination nanoparticle composition.

Antianxiety compounds: Alpha-casozepine (Zylkene), Ashwagandha, CBD, Chamomile, Gamma-aminobutyric acid (GABA), L-Theanine, L-Tryptophan, Licorice Root, Melatonin, Rhodiola Rosea, Valerian root.

Antioxidants: Alpha-Lipoic acid, Astaxanthin, Beta Alanine, Beta-carotene, Bladderwrack, Boswellia, Ceylon Cinnamon, Chondroitin sulfate, Coenzyme q10, Curcumin, Devil’s Claw, Eggshell Membrane, Flaxseed/Flaxseed oil, Ginger root extract, Glucosamine, Goji Berry, Guarana, Kelp seaweed, Krill oil, Lutein, Lycopene, Methylsulfonylmethane (MSM), Milk thistle, Monk Fruit Extract, Moringa, Peppermint oil, Pomegranate Extract, Quercetin, Selenium, Turmeric, Vitamin C, Vitamin E, Yucca root.

Compounds for Blood/Heart Health: Acetyl-L-carnitine, Bilberry extract, Coenzyme q10, Colostrum, Copper, Fish Oil, Ginger root extract, Hawthorn Berry Extract, Hesperidin Extract, Iron, Krill oil, L-Arginine, L-Carnitine, L-Taurine, Lycopene, Magnesium Oxide, Naringin Extract, Pomegranate Extract, Psyllium, Reishi Mushroom, Rosehip Extract, Vitamin B3 (Niacin), Vitamin B9 (Folic Acid), Vitamin B12, Vitamin D, Vitamin E, Vitamin K.

Compounds for Brain Health: Acetyl-L-carnitine, Alpha-Lipoic acid, Citicoline, Coenzyme q10, Copper, Eleuthero root (Siberian Ginseng), Fish Oil, Ginger root extract, Ginkgo Biloba, Gotu Kola, Green tea Extract, L-Arginine, L-Carnitine, L-Taurine, L-Tyrosine, Lion’s mane, Methylated Folate, Peppermint oil, Phosphatidylserine, Pomegranate Extract, Quercetin, Rhodiola Rosea, S-adenosyl-L-methionine, Selenium, Vitamin B1 (Thiamin), Vitamin B3 (Niacin), Vitamin B12, Vitamin D.

Compounds for Detox/Liver Health: Calcium, Choline, Glutathione, S-adenosyl-L-methionine, Vitamin B1 (Thiamin), Vitamin B3 (Niacin) , Vitamin B5 (Pantothenic acid), Vitamin B6, Vitamin B12, Vitamin C.

Digestive Aids: Aluminum hydroxide, Ginger root extract, Hawthorn Berry Extract, Jerusalem artichoke, L-Taurine, Lion’s mane, Magnesium Oxide, Peppermint oil, Psyllium, Pumpkin seed powder, S-adenosyl-L-methionine, Tree Barks Powder, Yucca root.

Compounds for Energy Boost: Acetyl-L-carnitine, Asian Ginseng, BCAAs, Beetroot Extract, Beta Alanine, Caterpillar fungus (Cordyceps sp.), Colostrum, Echinacea, Guarana, Iron, KSM-66 Ashwagandha , L-Carnitine, L-Taurine, Maca, Magnesium Oxide, Peppermint oil, Sodium Bicarbonate, Vitamin B1 (Thiamin) , Vitamin B2 (Riboflavin), Vitamin B3 (Niacin), Vitamin B5 (Pantothenic acid), Vitamin B6, Vitamin B9 (Folic Acid), Vitamin B12, Zinc.

Compounds for Eye Support: Bilberry extract, Goji Berry, Lutein, Vitamin A, Vitamin C, Vitamin D, Vitamin E, Zeaxanthin.

Compounds for Fur/Skin/Hair Health: Biotin, Collagen, Fern Extract, Fish oil, Fo-Ti Root, Hyaluronic Acid, Keratin, Omega3/Algal Oil, Peppermint oil, Primrose Oil, Saw Palmetto, Selenium, Sunflower Oil, Turmeric, Vitamin A, Vitamin B5 (Pantothenic acid), Vitamin D, Vitamin E, Zinc.

Compounds for Immune Support & Anti-allergy: Astaxanthin, Beetroot Extract, Beta Alanine, Colostrum, Copper, Echinacea, Fish Oil, Goji Berry, Iron, Magnesium Oxide, Milk thistle, Monk Fruit Extract, Nettle root extract, Pineapple powder, Quercetin, Turmeric, Vitamin A, Vitamin C, Vitamin D, Vitamin E, Yucca Root, Zinc.

Compounds for Kidney/Urinary Tract Health: Aluminum hydroxide, Astragalus root powder, Choline, Dong Quai Root, Nettle root extract, Vitamin B9 (Folic Acid).

Compounds for Pain Relief: CBD, Chondroitin sulfate, Eggshell Membrane, Fish Oil, Glucosamine, Hyaluronic Acid, Krill oil, Methylsulfonylmethane (MSM), S-adenosyl-L-methionine.

Vitamins/Minerals: beta-carotene, Calcium, Chromium, Copper, Iron, Magnesium, Manganese, Potassium, Sodium Bicarbonate, Vitamin A, Vitamin B1 (Thiamin), Vitamin B2 (Riboflavin), Vitamin B3 (Niacin), Vitamin B5 (Pantothenic acid), Vitamin B6, Vitamin B9 (Folic Acid), Vitamin B12, Vitamin C, Vitamin D, Vitamin E, Vitamin K, Zinc.

Compounds for Weight Loss/Appetite Control: Caralluma Fimbriata, Choline, CLA, Colostrum, Garcinia Cambodia, Guarana, L-Carnitine, Psyllium.

According to one aspect, disclosed is a therapeutic agent or nutraceutical composition consisting essentially of a sugar, sugar alcohol, or sugar substitute; an effective amount of the therapeutic agent or nutraceutical; wherein the therapeutic agent or nutraceutical complex, when present, comprises less than 25% water by weight of the therapeutic agent or nutraceutical complex; and wherein the therapeutic agent or nutraceutical syrup, when present, comprises 30% to 60% water by weight of the therapeutic agent or nutraceutical syrup. In a still further aspect, the therapeutic agent or nutraceutical composition consists of the sugar, sugar alcohol, or sugar substitute; the effective amount of the therapeutic agent or nutraceutical water; wherein the therapeutic agent or nutraceutical complex, when present, comprises less than 25% water by weight of the therapeutic agent or nutraceutical complex; and wherein the therapeutic agent or nutraceutical syrup, when present, comprises 30% to 60% water by weight of the therapeutic agent or nutraceutical syrup. In a further aspect, the therapeutic agent or nutraceutical complex, when present, comprises 10% to 20% water by weight of the therapeutic agent or nutraceutical complex. In another aspect, the therapeutic agent or nutraceutical complex, when present, comprises 11% to 17% water by weight of the therapeutic agent or nutraceutical complex. Also disclosed are products comprising the therapeutic agent or nutraceutical composition.

### I. Acceptable Salts and Carriers

In some aspects, a naturally-occurring therapeutic agent or nutraceutical or sugar present in the dispersion or a product prepared therefrom can be present as an acceptable, non-naturally occurring salt. Thus, for example, a naturally-occuring therapeutic agent or nutraceutical or sugar present in the composition can be present as a non-naturally occurring acid or base salt of the naturally-occurring therapeutic agent or nutraceutical or sugar. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, and quaternary ammonium (methyl iodide, ethyl iodide, and the like) salts.

Acceptable salts can be prepared by reaction of the therapeutic agent or nutraceutical or sugar with a mineral or organic acid or an inorganic base, such as salts including sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, β-hydroxybutyrates, glycolates, tartrates, methane-sulfonates, propanesulfonates, naphthalene-1-sulfonates, naphthalene-2-sulfonates, and mandelates.

According to one aspect, if the therapeutic agent or nutraceutical or sugar has one or more acidic functional groups, the desired salt can be prepared by any suitable method known in the art, for example, treatment of the free acid with an inorganic or organic base, such as an amine (primary, secondary or tertiary), an alkali metal hydroxide or alkaline earth metal hydroxide, or the like. It is understood that the acceptable salts are non-toxic and suitable for ingestion. Additional information on suitable acceptable salts can be found in Remington’s Pharmaceutical Sciences, 17th ed., Mack Publishing Company, Easton, Pa., 1985, which is incorporated herein by reference.

In some aspects, a product prepared from a disclosed dispersion can comprise a naturally-occurring therapeutic agent or nutraceutical and/or sugar present along with an acceptable, non-naturally occurring carrier. Various suitable non-naturally occurring carriers are described in Remington’s Pharmaceutical Sciences, 17th ed., Mack Publishing Company, Easton, Pa., 1985, which is incorporated herein by reference. Non-limiting examples include non-naturally occurring polymeric carriers or binders in liquid or solid form, such as polyglycolic acids, synthetic polymers, non-naturally occurring conjugates of proteins, and the like.

### II. Exemplary Dispersions With APIs/Nutraceuticals

Specific, non-limiting examples of dispersions useful for preparing the therapeutic agent or nutraceutical composition include those listed in Table 2. The compositions listed in Table 2 include dispersions comprising a sugar, sugar alcohol, or sugar substitute selected from a sugar comprising sucrose, fructose, and glucose (e.g., cane sugar or organic cane sugar); a sugar alcohol selected from erythritol or xylitol; maltodextrin, or sucralose. In one aspect, the exemplary dispersion compositions listed in Table 2 include a sugar comprising sucrose, fructose, and glucose such as cane sugar or organic cane sugar.

Exemplary Dispersion Compositions

| Mass Ratio of Cargo Entity to Sugar / Sugar Alcohol / Sugar Substitute | Mass Ratio of Sugar / Sugar Alcohol / Sugar Substitute to Water |
|---|---|
| 1:300 to 1:3 | 1:8 to 1:1 |
| 1:300 to 1:3 | 1:8 to 1:1.2 |
| 1:300 to 1:3 | 1:8 to 1:1.3 |
| 1:300 to 1:3 | 1:8 to 1:1.5 |
| 1:300 to 1:3 | 1:6 to 1:4 |
| 1:300 to 1:3 | 1:4 |
| 1:50 to 1:3 | 1:8 to 1:1 |
| 1:50 to 1:3 | 1:8 to 1:1.2 |
| 1:50 to 1:10 | 1:8 to 1:1 |
| 1:50 to 1:10 | 1:8 to 1:1.2 |
| 1:40 to 1:10 | 1:8 to 1:1 |
| 1:40 to 1:10 | 1:8 to 1:1.2 |
| 1:30 to 1:10 | 1:8 to 1:1 |
| 1:30 to 1:10 | 1:8 to 1:1.2 |
| 1:30 to 1:10 | 1:8 to 1:1.2 |
| 1:30 to 1:15 | 1:8 to 1:1 |
| 1:30 to 1:15 | 1:8 to 1:1.2 |
| 1:20 | 1:8 to 1:1 |
| 1:20 | 1:8 to 1:1.2 |
| 1:50 to 1:10 | 1:8 to 1:1.3 |
| 1:50 to 1:10 | 1:8 to 1:1.5 |
| 1:50 to 1:10 | 1:6 to 1:4 |
| 1:50 to 1:10 | 1:4 |
| 1:30 to 1:10 | 1:8 to 1:1.3 |
| 1:30 to 1:10 | 1:8 to 1:1.5 |
| 1:30 to 1:10 | 1:6 to 1:4 |
| 1:30 to 1:10 | 1:4 |
| 1:30 to 1:15 | 1:8 to 1:1.3 |
| 1:30 to 1:15 | 1:8 to 1:1.5 |
| 1:30 to 1:15 | 1:6 to 1:4 |
| 1:30 to 1:15 | 1:4 |
| 1:20 | 1:8 to 1:1.3 |
| 1:20 | 1:8 to 1:1.5 |
| 1:20 | 1:6 to 1:4 |
| 1:20 | 1:4 |

### I. Other Cargo Entities

In some aspects, the disclosed dispersion can comprise additional ingredients that can be encapsulated with the nanoparticle, along with the cargo entity. In one aspect, the dispersion comprises a nutraceutical. Any suitable nutraceutical additive can be present in the dispersion. In some aspects, the nutraceutical additive can be Vitamin E, Echinacea, St. John’s Wart, Kava Tincture, Ginseng, Black Seed Oil, Milk Thistle, Turmeric, Blank-Nega, Valerian root extract, among others. In a further aspect, the dispersion can comprise a suitable flavor additive such as a natural or artificial flavoring.

In some aspects, the disclosed compositions and nanoparticles can comprise further additives. Such additives can be added during the encapsulation process, in which the further additive(s) can be potentially encapsulated, or such additives can be mixed with the final encapsulated product. Alternatively, one or more further additives can be encapsulated using a disclosed process and then mixed with cargo entity-encapsulated nanoparticles. Such further additives include Ginger, Lemongrass, Yucca extract, Orris root, Black Tea, Green Tea, Other tea extracts or powders like Matcha powder and the like, natural essential oils and oil extracts used in the food and/or pharmaceutical industry, Food-grade nut extracts, Olive oil such as Extra virgin olive oil, Coconut oil, Lime and/or lemon extract, Fruit and berry extracts and fruit/berry dry powders, Flower extracts and Flower dry powders, Cinnamon, Cumin, and other spices, Soya Lecithin and other food-grade surfactants/emulsifiers, Guar Gum, β-glucans and other sugars, Malt extract, Mowrah butter, Kelp powder, Astaxanthin and other carotenoids, Natural Food supplements and flavorings, Coffee extract, Cocoa extract, water-soluble vitamins (Thiamine, Riboflavin, Pantothenic acid, Niacin, Pyridoxine, Folcin or Folic acid, Cyanocobalamin, Ascorbic Acid, among others), and multivitamin mixtures.

Other cargo entities include any of those listed below in Tables 2-57, in the amounts listed or in other suitable amounts.

In one aspect, the composition comprises the following cargo entities, each of which can be encapsulated by the same or a different carrier: Beet Root (Organic), Ginger Root, Ceylon Cinnamon, Beta Carotene (Vitamin A), Thiamine (Vitamin B1), Riboflavin (Vitamin B2), Pantothenic Acid (Vitamin B5), Pyridoxine (Vitamin B6), Biotin (Vitamin B7), Folate or Folic Acid (Vitamin B9), Cobalamin (Vitamin B12), Vitamin D3, Vitamin E, Sodium Bicarbonate, Selenium, Zinc (Zinc Oxide), Calcium Lactate, Iron (Ferrous Fumarate), Magnesium Oxide, and Potassium Citrate. In a further aspect, each of the foregoing cargo entities can be encapsulated in a separate nanoparticle carrier and combined together in a powdered composition suitable for incorporation into a food or beverage product.

In one aspect, the composition comprises the following cargo entities, each of which can be encapsulated by the same or a different carrier: Asian Ginseng, Beet Root, Caralluma Fimbriata, Cannabidiol (CBD), Ceylon Cinnamon, Conjugated linoleic acid (CLA), Coenzyme Q10 (CoQ10), Curcumin, Garcinia Cambogia, Ginger Root, Guarana, KSM-66 Ashwagandha, L-Arginine, L-Carnitine, L-Theanine, Magnesium Oxide, Moringa, Methylsulfonylmethane (MSM), Turmeric, Beta Hydroxy Beta, Methylbutyrate, Peppermint oil, Vitamin D3, and Beta Alanine. In a further aspect, each of the foregoing cargo entities can be encapsulated in a separate nanoparticle carrier and combined together in a powdered composition suitable for incorporation into a food or beverage product.

In one aspect, the composition comprises the following cargo entities, each of which can be encapsulated by the same or a different carrier: Lisinopril, Hydrochlorothiazide, Atenolol, Irbesartan, and Citalopram. In a further aspect, each of the foregoing cargo entities can be encapsulated in a separate nanoparticle carrier and combined together in a powdered composition suitable for incorporation into a food, beverage, or pharmaceutical product.

In one aspect, the composition comprises the following cargo entities, each of which can be encapsulated by the same or a different carrier: Ethyl estradiol, Vitamin D3, Pyroxidine (Vit B6), and Lime Doterra oil. In a further aspect, each of the foregoing cargo entities can be encapsulated in a separate nanoparticle carrier and combined together in a powdered composition suitable for incorporation into a food, beverage, or pharmaceutical product.

In one aspect, the composition comprises the following cargo entities, each of which can be encapsulated by the same or a different carrier: Erythromycin, Guaifenesin, Echinacea, Turmeric, and Prednisone. In a further aspect, each of the foregoing cargo entities can be encapsulated in a separate nanoparticle carrier and combined together in a powdered composition suitable for incorporation into a food, beverage, or pharmaceutical product.

In one aspect, the composition comprises the following cargo entities, each of which can be encapsulated by the same or a different carrier: Moringa, CBD, Choline Bitartrate, and Folic Acid. In a further aspect, each of the foregoing cargo entities can be encapsulated in a separate nanoparticle carrier and combined together in a powdered composition suitable for incorporation into a food, beverage, or pharmaceutical product.

In one aspect, the composition comprises the following cargo entities, each of which can be encapsulated by the same or a different carrier: Maca, Ceylon Cinnamon, Clove Oil, Folic Acid, and Vitamin C. In a further aspect, each of the foregoing cargo entities can be encapsulated in a separate nanoparticle carrier and combined together in a powdered composition suitable for incorporation into a food, beverage, or pharmaceutical product.

In one aspect, the composition comprises the following cargo entities, each of which can be encapsulated by the same or a different carrier: Ceylon Cinnamon, KSM-66, Tumeric, and Magnesium Oxide. In a further aspect, each of the foregoing cargo entities can be encapsulated in a separate nanoparticle carrier and combined together in a powdered composition suitable for incorporation into a food, beverage, or pharmaceutical product.

In one aspect, the composition comprises the following cargo entities, each of which can be encapsulated by the same or a different carrier: Folic Acid, L-Theanine, Asian Ginseng, and Nanoparticle Encapsulated Hop Oil. In a further aspect, each of the foregoing cargo entities can be encapsulated in a separate nanoparticle carrier and combined together in a powdered composition suitable for incorporation into a food, beverage, or pharmaceutical product.

In one aspect, the composition comprises the following cargo entities, each of which can be encapsulated by the same or a different carrier: Caralluma Fimbriata, Milk Thistle, Beet Root, Selenium, and Nanoparticle-encapsulated bacon grease. In a further aspect, each of the foregoing cargo entities can be encapsulated in a separate nanoparticle carrier and combined together in a powdered composition suitable for incorporation into a food, beverage, or pharmaceutical product.

In one aspect, the composition comprises the following cargo entities, each of which can be encapsulated by the same or a different carrier: Turmeric, Flaxseed Oil, Glucosamine, Yucca Root, Kelp Seaweed, Quercetin, Coenzyme Q10, and Lipoic Acid. In a further aspect, each of the foregoing cargo entities can be encapsulated in a separate nanoparticle carrier and combined together in a powdered composition suitable for incorporation into a food, beverage, or pharmaceutical product.

In one aspect, the composition comprises the following cargo entities, each of which can be encapsulated by the same or a different carrier: Tree Barks Powder, Psyllium, and Ginger Root Extract. In a further aspect, each of the foregoing cargo entities can be encapsulated in a separate nanoparticle carrier and combined together in a powdered composition suitable for incorporation into a food, beverage, or pharmaceutical product.

In one aspect, the composition comprises the following cargo entities, each of which can be encapsulated by the same or a different carrier: Collagen, Biotin, Vitamin E, Vitamin D3, Folate, and Beta Carotene. In a further aspect, each of the foregoing cargo entities can be encapsulated in a separate nanoparticle carrier and combined together in a powdered composition suitable for incorporation into a food, beverage, or pharmaceutical product.

In one aspect, the composition comprises the following cargo entities, each of which can be encapsulated by the same or a different carrier: Betaine, Thiamine (Vitamin B1), Riboflavin (Vitamin B2), Pantothenic Acid (Vitamin B5), Pyridoxine (Vitamin B6), Biotin (Vitamin B7), Folate or Folic Acid (Vitamin B9), Cobalamin (Vitamin B12), Choline, Turmeric, Asian Ginseng, Guarana, Sodium Chloride, Potassium Citrate, Vitamin C, Milk Thistle, and Glutathione. In a further aspect, each of the foregoing cargo entities can be encapsulated in a separate nanoparticle carrier and combined together in a powdered composition suitable for incorporation into a food, beverage, or pharmaceutical product.

In one aspect, the composition comprises the following cargo entities, each of which can be encapsulated by the same or a different carrier: Amber Liquid Extract (12.5 SRM), Pale Liquid Extract (8.0 SRM), Caramel Crystal Malt -60 ml(60 SRM), Nanoparticle Encapsulated Hop Oil (45 min), Nanoparticle Encapsulated Hop Oil, Honey (Boil 45 min), and Munton Fison Ale (Munton Fison # -). In a further aspect, each of the foregoing cargo entities can be encapsulated in a separate nanoparticle carrier and combined together in a powdered composition suitable for incorporation into a food or beverage product.

In one aspect, the composition comprises the following cargo entities, each of which can be encapsulated by the same or a different carrier: Asian Ginseng, Beet Root, Caralluma Fimbriata, Cannabidiol (CBD), Ceylon Cinnamon, Conjugated linoleic acid (CLA), Coenzyme Q10 (CoQ10), Curcumin, Garcinia Cambogia, Ginger Root, Guarana, Ashwagandha , L-Arginine, L-Carnitine, L-Theanine, Sodium Bicarbonate, Magnesium Oxide, Moringa, Methylsulfonylmethane (MSM), Turmeric, Beta Hydroxy Beta-Methylbutyrate, Peppermint oil, Vitamin D, and Beta Alanine. In a further aspect, each of the foregoing cargo entities can be encapsulated in a separate nanoparticle carrier and combined together in a powdered composition suitable for incorporation into a food or beverage product.

In one aspect, the composition comprises the following cargo entities, each of which can be encapsulated by the same or a different carrier: Beet Root, Ginger Root, Ceylon Cinnamon, Beta Carotene, Vitamin B1 (Thiamin), Vitamin B2 (Riboflavin), Vitamin B5 (Pantothenic acid), Vitamin B6, Vitamin B7 (Biotin), Vitamin B9 (Folic Acid), Vitamin B12, Vitamin D, Vitamin E, Sodium Bicarbonate, Selenium, Zinc, Calcium Lactate, Iron, Magnesium Oxide, and Potassium Citrate. In a further aspect, each of the foregoing cargo entities can be encapsulated in a separate nanoparticle carrier and combined together in a powdered composition suitable for incorporation into a food or beverage product.

In one aspect, the composition comprises the following cargo entities, each of which can be encapsulated by the same or a different carrier: Betaine, L-Taurine, Ginger root extract, S-adenosyl-L-methionine, Thiamine (Vitamin B1), Riboflavin (Vitamin B2), Vitamin B5 (Pantothenic acid), Vitamin B6, Vitamin B7 (Biotin), Vitamin B9 (Folic Acid), Vitamin B12, Choline, Turmeric, Asian Ginseng, Guarana, Sodium Chloride, Potassium Citrate, Vitamin C, Milk Thistle, and Glutathione. In a further aspect, each of the foregoing cargo entities can be encapsulated in a separate nanoparticle carrier and combined together in a powdered composition suitable for incorporation into a food or beverage product.

In one aspect, the composition comprises the following cargo entities, each of which can be encapsulated by the same or a different carrier: Vitamin A, Vitamin C, Vitamin E, Coenzyme Q10, Vitamin B7 (Biotin), Selenium, Manganese, Omega-3, Omega-6, Flaxseed Oil, Zinc, Fern Extract, Collagen, Keratin, Saw Palmetto, Peppermint Oil, Astaxanthin, and Iron. In a further aspect, each of the foregoing cargo entities can be encapsulated in a separate nanoparticle carrier and combined together in a powdered composition suitable for incorporation into a food or beverage product.

In one aspect, the composition comprises the following cargo entities, each of which can be encapsulated by the same or a different carrier: Reishi Mushroom, Vitamin A, Vitamin B1 (Thiamin), Vitamin B2 (Riboflavin), Vitamin B3 (Niacin), Vitamin B5 (Pantothenic acid), Vitamin B6, Vitamin B9 (Folic Acid), Vitamin B12, Vitamin C, Vitamin D, Vitamin E, Chromium, Copper, Iron, Magnesium Oxide, Selenium, Zinc, Potassium Citrate, Vitamin K, Monk Fruit Extract, and L-Leucine. In a further aspect, each of the foregoing cargo entities can be encapsulated in a separate nanoparticle carrier and combined together in a powdered composition suitable for incorporation into a food or beverage product.

In one aspect, the composition comprises the following cargo entities, each of which can be encapsulated by the same or a different carrier: Calcium Lactate, Chromium, Iron, Magnesium Oxide, Manganese, Potassium Citrate, Vitamin A, Vitamin B1 (Thiamin), Vitamin B2 (Riboflavin), Vitamin B3 (Niacin), Vitamin B6, Vitamin B12, Vitamin C, Vitamin D, Vitamin K, Zinc, Omega-3, Selenium, Methylated Folate, Lycopene, Lutein, Coenzyme Q10, and Astaxanthin. In a further aspect, each of the foregoing cargo entities can be encapsulated in a separate nanoparticle carrier and combined together in a powdered composition suitable for incorporation into a food or beverage product.

In one aspect, the composition comprises the following cargo entities, each of which can be encapsulated by the same or a different carrier: Magnesium Oxide, Vitamin B12, Vitamin D, Omega-3, L-Theanine, Ashwagandha, Rhodiola Rosea, Gamma-aminobutyric acid (GABA), Passionflower, Valerian Root, and Licorice Root. In a further aspect, each of the foregoing cargo entities can be encapsulated in a separate nanoparticle carrier and combined together in a powdered composition suitable for incorporation into a food or beverage product.

In one aspect, the composition comprises the following cargo entities, each of which can be encapsulated by the same or a different carrier: L-Taurine, S-adenosyl-L-methionine, Calcium, Lactate, Acetyl-L-carnitine, Vitamin D, Vitamin B6, Vitamin B12, Beta-Carotene, Selenium, Ginkgo Biloba, Vitamin E, Collagen, Omega-3, Collagen, Peppermint Oil, Citicoline, Coenzyme Q10, Phosphatidylserine, Saw Palmetto, and Curcumin. In a further aspect, each of the foregoing cargo entities can be encapsulated in a separate nanoparticle carrier and combined together in a powdered composition suitable for incorporation into a food or beverage product.

In one aspect, the composition comprises the following cargo entities, each of which can be encapsulated by the same or a different carrier: S-adenosyl-L-methionine, Pomegranate Extract, Rhodiola Rosea, L-Tyrosine, Vitamin B1 (Thiamin), Vitamin B6, Vitamin B9 (Folic Acid), Vitamin B12, Gingko Biloba, Omega-3, Creatine, Citocoline, Phosphatidylserine, Acetyl-L-carnitine, Green Tea Extract (Source of Caffeine), and Bacopa Monnieri. In a further aspect, each of the foregoing cargo entities can be encapsulated in a separate nanoparticle carrier and combined together in a powdered composition suitable for incorporation into a food or beverage product.

In one aspect, the composition comprises the following cargo entities, each of which can be encapsulated by the same or a different carrier: S-adenosyl-L-methionine, Glucosamine, Chondroitin Sulfate, Hyaluronic Acid, Collagen, MSM, Curcumin, Devil’s Claw, Turmeric, Keratin, Peppermint Oil, Hops Flowers (Humulus Lupulus), Boswellia, Omega-3, and Krill Oil. In a further aspect, each of the foregoing cargo entities can be encapsulated in a separate nanoparticle carrier and combined together in a powdered composition suitable for incorporation into a food or beverage product.

In one aspect, the composition comprises the following cargo entities, each of which can be encapsulated by the same or a different carrier: Vitamin B6, Vitamin C, Vitamin E, Vitamin B7 (Biotin), Gotu Kola, Goji Berry, Fo-Ti Root, Bladderwrack, Nori Yaki, MSM, Collagen, Keratin, Hyaluronic Acid, Astaxanthin, Calcium, Peppermint Oil, Zinc, Selenium, Reishi Mushroom, Copper, and Manganese. In a further aspect, each of the foregoing cargo entities can be encapsulated in a separate nanoparticle carrier and combined together in a powdered composition suitable for incorporation into a food or beverage product.

In one aspect, the composition comprises the following cargo entities, each of which can be encapsulated by the same or a different carrier: Aluminum hydroxide, Magnesium, Licorice Root, Peppermint Oil, Chamomile, Ginger root extract, L-Taurine, Psyllium, Yucca root, and S-adenosyl-L-methionine. In a further aspect, each of the foregoing cargo entities can be encapsulated in a separate nanoparticle carrier and combined together in a powdered composition suitable for incorporation into a food or beverage product.

In one aspect, the composition comprises the following cargo entities, each of which can be encapsulated by the same or a different carrier: Colostrum, Calcium, Vitamin D, Vitamin K, L-Arginine, Zinc, Omega-3/Fish Oils, Beetroot Extract, Phosphatidylserine, Iron, Vitamin A, Ginseng, Collagen Hydrolysate, Iodine, and Gingko Biloba. In a further aspect, each of the foregoing cargo entities can be encapsulated in a separate nanoparticle carrier and combined together in a powdered composition suitable for incorporation into a food or beverage product.

In one aspect, the composition comprises the following cargo entities, each of which can be encapsulated by the same or a different carrier: Peppermint Oil, L-Carnitine, Ashwagandha, Rhodiola Rosea, Vitamin B12, Iron, Coenzyme Q10 (CoQ10), Creatine, Beta Alanine, L-Citrulline, Beetroot Extract, L-Theanine, Tyrosine, and Green Tea Extract. In a further aspect, each of the foregoing cargo entities can be encapsulated in a separate nanoparticle carrier and combined together in a powdered composition suitable for incorporation into a food or beverage product.

In one aspect, the composition comprises the following cargo entities, each of which can be encapsulated by the same or a different carrier: Pomegranate Extract, Goji Berry, Peppermint Oil, Echinacea, Beetroot Extract, Astaxanthin, Zinc, Iron, Magnesium, Quercetin, Vitamin A, Vitamin C, Vitamin D, and Vitamin E. In a further aspect, each of the foregoing cargo entities can be encapsulated in a separate nanoparticle carrier and combined together in a powdered composition suitable for incorporation into a food or beverage product.

In one aspect, the composition comprises the following cargo entities, each of which can be encapsulated by the same or a different carrier: Goji Berry, Peppermint Oil, Echinacea, Grape Seed Extract, Pycnogenol, Goldenseal, Spirulina, Quercetin, Vitamin C, and Omega 3. In a further aspect, each of the foregoing cargo entities can be encapsulated in a separate nanoparticle carrier and combined together in a powdered composition suitable for incorporation into a food or beverage product.

In one aspect, the composition comprises the following cargo entities, each of which can be encapsulated by the same or a different carrier: Goji Berry, Bilberry Extract, Beta-Carotene, Vitamin A, Vitamin C, Vitamin D, Vitamin E, Lutein, and Zeaxanthin. In a further aspect, each of the foregoing cargo entities can be encapsulated in a separate nanoparticle carrier and combined together in a powdered composition suitable for incorporation into a food or beverage product.

In one aspect, the composition comprises the following cargo entities, each of which can be encapsulated by the same or a different carrier: Choline, CLA, Guarana, Garcinia Cambodia, Psyllium, Collagen, Omega 3, and L-Carnitine. In a further aspect, each of the foregoing cargo entities can be encapsulated in a separate nanoparticle carrier and combined together in a powdered composition suitable for incorporation into a food or beverage product.

In one aspect, the composition comprises the following cargo entities, each of which can be encapsulated by the same or a different carrier: Whey Protein, Creatine, Beta Alanine, Sunflower Oil, Monk Fruit, Beta Hydroxy Beta-Methylbutyrate, and Ginger Root. In a further aspect, each of the foregoing cargo entities can be encapsulated in a separate nanoparticle carrier and combined together in a powdered composition suitable for incorporation into a food or beverage product.

In one aspect, the composition comprises the following cargo entities, each of which can be encapsulated by the same or a different carrier: L-Taurine, Pomegranate Extract, Omega-3, Bilberry Extract, Magnesium, Vitamin B3 (Niacin), Vitamin B9 (Folic Acid), Vitamin B12, Vitamin E, and Coenzyme Q10. In a further aspect, each of the foregoing cargo entities can be encapsulated in a separate nanoparticle carrier and combined together in a powdered composition suitable for incorporation into a food or beverage product.

In one aspect, the composition comprises the following cargo entities, each of which can be encapsulated by the same or a different carrier: Aluminum hydroxide, Astragalus root powder, Choline, Dong Quai Root, Nettle root extract, Vitamin B9 (Folic Acid), and Iron. In a further aspect, each of the foregoing cargo entities can be encapsulated in a separate nanoparticle carrier and combined together in a powdered composition suitable for incorporation into a food or beverage product.

In one aspect, the composition comprises the following cargo entities, each of which can be encapsulated by the same or a different carrier: Iodine, Iron, Zinc, Vitamin B1 (Thiamin), Vitamin B3 (Niacin), Vitamin B6, Vitamin B9 (Folic Acid), Vitamin B12, Vitamin D, Vitamin A, Vitamin C, Vitamin E, DHA, Choline, Calcium, and Magnesium. In a further aspect, each of the foregoing cargo entities can be encapsulated in a separate nanoparticle carrier and combined together in a powdered composition suitable for incorporation into a food or beverage product.

In one aspect, the composition comprises the following cargo entities, each of which can be encapsulated by the same or a different carrier: S-adenosyl-L-methionine, Vitamin B1 (Thiamin), Vitamin B2 (Riboflavin), Vitamin B3 (Niacin), Vitamin B5 (Pantothenic acid), Vitamin B6, Vitamin B9 (Folic Acid), Vitamin B12, Vitamin C, Black Pepper Extract, Asian Ginseng, Calcium, Magnesium, and Beta-Carotene. In a further aspect, each of the foregoing cargo entities can be encapsulated in a separate nanoparticle carrier and combined together in a powdered composition suitable for incorporation into a food or beverage product.

In one aspect, the composition comprises the following cargo entities, each of which can be encapsulated by the same or a different carrier: Vitamin B3 (Niacin), Hops Flowers (Humulus Lupulus), Ginko Biloba, Mucuna Pruruiens, Epimedium Sagittat (Horny Goat Weed), Cayenne Extract, Black Pepper Extract, Ashwagandha, Damiana Extract, Maca, and L-Arginine. In a further aspect, each of the foregoing cargo entities can be encapsulated in a separate nanoparticle carrier and combined together in a powdered composition suitable for incorporation into a food or beverage product.

In one aspect, the composition comprises the following cargo entities, each of which can be encapsulated by the same or a different carrier: Yohimbe, Vitamin B3 (Niacin), L-Arginine, Ginko Biloba, and Epimedium Sagittat (Horny Goat Weed). In a further aspect, each of the foregoing cargo entities can be encapsulated in a separate nanoparticle carrier and combined together in a powdered composition suitable for incorporation into a food or beverage product.

In one aspect, the composition comprises the following cargo entities, each of which can be encapsulated by the same or a different carrier: L-Theanine, Tyrosine, Omega 3-Fatty Acids, Tryptophan, Vitamin B1 (Thiamin), Vitamin B2 (Riboflavin), Vitamin B3 (Niacin), Vitamin B5 (Pantothenic acid), Vitamin B6, Vitamin B9 (Folic Acid), Vitamin B12, Vitamin C, Vitamin D, Vitamin E, Ginko Biloba, Bacopa Monnieri, Resveratrol, Green Tea Extract, Ashwagandha, Choline, Creatine, Theacrine, Zinc, Asian Ginseng, Curcumin, L-Leucine, and Rhodiola Rosea. In a further aspect, each of the foregoing cargo entities can be encapsulated in a separate nanoparticle carrier and combined together in a powdered composition suitable for incorporation into a food or beverage product.

In one aspect, the composition comprises the following cargo entities, each of which can be encapsulated by the same or a different carrier: Fish Oil, Vitamin A, Vitamin B5 (Pantothenic acid), Vitamin D, Vitamin E, Chamomile, Primrose oil, Sunflower oil, Zinc, Turmeric, Hyaluronic Acid, and Vitamin B7 (Biotin). In a further aspect, each of the foregoing cargo entities can be encapsulated in a separate nanoparticle carrier and combined together in a powdered composition suitable for incorporation into a food or beverage product.

In one aspect, the composition comprises the following cargo entities, each of which can be encapsulated by the same or a different carrier: Glucosamine, Coenzyme Q10, Vitamin B1 (Thiamin), Vitamin B2 (Riboflavin), Vitamin B3 (Niacin), Vitamin B6, Vitamin B12, Vitamin C, Vitamin E, B-Carotene, Milk Thistle, Ginger Root Extract, Vitamin D, Fish Oil, and CBD. In a further aspect, each of the foregoing cargo entities can be encapsulated in a separate nanoparticle carrier and combined together in a powdered composition suitable for incorporation into a food or beverage product.

In one aspect, the composition comprises the following cargo entities, each of which can be encapsulated by the same or a different carrier: L-Trytophan, Alpha-Casozephine (Zylkene), Chamomile, CBD, Melatonin, L-Theanine, and Valerian Root. In a further aspect, each of the foregoing cargo entities can be encapsulated in a separate nanoparticle carrier and combined together in a powdered composition suitable for incorporation into a food or beverage product.

In one aspect, the composition comprises the following cargo entities, each of which can be encapsulated by the same or a different carrier: Vitamin A, Vitamin B1 (Thiamin), Vitamin B2 (Riboflavin), Vitamin B6, Vitamin B12, Vitamin C, Vitamin E, B-Carotene, alpha-lipoic acid, L-Taurine, L-Carnitine, S-adenosyl-L-methionine, Turmeric, CBD, Coenzyme Q10, Pomegranate Extract, and Quercetin. In a further aspect, each of the foregoing cargo entities can be encapsulated in a separate nanoparticle carrier and combined together in a powdered composition suitable for incorporation into a food or beverage product.

In one aspect, the composition comprises the following cargo entities, each of which can be encapsulated by the same or a different carrier: Vitamin B1 (Thiamin), Vitamin B2 (Riboflavin), Vitamin B3 (Niacin), Vitamin B6, Vitamin B12, Vitamin C, Vitamin E, Fish Oil, L-Arginine, S-adenosyl-L-methionine, Siberian Ginseng Extract, Pomegranate Extract, and Selenium. In a further aspect, each of the foregoing cargo entities can be encapsulated in a separate nanoparticle carrier and combined together in a powdered composition suitable for incorporation into a food or beverage product.

In one aspect, the composition comprises the following cargo entities, each of which can be encapsulated by the same or a different carrier: Glucosamine, S-adenosyl-L-methionine, MSM, Chondroitin sulfate, CBD, Fish Oil, Krill oil, and Hyaluronic Acid. In a further aspect, each of the foregoing cargo entities can be encapsulated in a separate nanoparticle carrier and combined together in a powdered composition suitable for incorporation into a food or beverage product.

In one aspect, the composition comprises the following cargo entities, each of which can be encapsulated by the same or a different carrier: Ginger Root Extract, Magnesium Oxide, Turmeric, Hawthorn Berry Extract, Jerusalem artichoke, Yucca Root, Aluminum hydroxide, and Pumpkin seed powder. In a further aspect, each of the foregoing cargo entities can be encapsulated in a separate nanoparticle carrier and combined together in a powdered composition suitable for incorporation into a food or beverage product.

In one aspect, the composition comprises the following cargo entities, each of which can be encapsulated by the same or a different carrier: Vitamin A, Vitamin C, Vitamin E, Pomegranate Extract, Beetroot Extract, Zinc, Echinacea, Iron, Milk Thistle, Colostrum, Fish Oil, Magnesium Oxide, and Quercetin. In a further aspect, each of the foregoing cargo entities can be encapsulated in a separate nanoparticle carrier and combined together in a powdered composition suitable for incorporation into a food or beverage product.

In one aspect, the composition comprises the following cargo entities, each of which can be encapsulated by the same or a different carrier: Vitamin A, Yucca Root, Turmeric, Fish Oil, Echinacea, Colostrum, Nettle root extract, and Quercetin. In a further aspect, each of the foregoing cargo entities can be encapsulated in a separate nanoparticle carrier and combined together in a powdered composition suitable for incorporation into a food or beverage product.

In one aspect, the composition comprises the following cargo entities, each of which can be encapsulated by the same or a different carrier: Vitamin A, Vitamin B1 (Thiamin), Vitamin B2 (Riboflavin), Vitamin B3 (Niacin), Vitamin B6, Vitamin B12, Vitamin D, Magnesium Oxide, Ashwagandha, Curcumin, BCAAs, and Zinc. In a further aspect, each of the foregoing cargo entities can be encapsulated in a separate nanoparticle carrier and combined together in a powdered composition suitable for incorporation into a food or beverage product.

In one aspect, the composition comprises the following cargo entities, each of which can be encapsulated by the same or a different carrier: Vitamin A, Vitamin C, Lutein, Zeaxanthin, Bilberry extract, and Vitamin E. In a further aspect, each of the foregoing cargo entities can be encapsulated in a separate nanoparticle carrier and combined together in a powdered composition suitable for incorporation into a food or beverage product.

In one aspect, the composition comprises the following cargo entities, each of which can be encapsulated by the same or a different carrier: Fish Oil, L-Carnitine, L-Arginine, Choline, CLA, Pysillium, Caralluma Fibriata, Garcinia Cambogia, and CBD. In a further aspect, each of the foregoing cargo entities can be encapsulated in a separate nanoparticle carrier and combined together in a powdered composition suitable for incorporation into a food or beverage product.

In one aspect, the composition comprises the following cargo entities, each of which can be encapsulated by the same or a different carrier: Vitamin B12, Vitamin D, Siberian Ginseng Extract, L-Taurine, Calcium Lactate, and Zinc. In a further aspect, each of the foregoing cargo entities can be encapsulated in a separate nanoparticle carrier and combined together in a powdered composition suitable for incorporation into a food or beverage product.

In one aspect, the composition comprises the following cargo entities, each of which can be encapsulated by the same or a different carrier: Iron, Zinc, Calcium Lactate, Magnesium Oxide, Vitamin A, Vitamin B1 (Thiamin), Vitamin B2 (Riboflavin), Vitamin B3 (Niacin), Vitamin B5 (Pantothenic acid), Vitamin B6, Vitamin B9 (Folic Acid), Vitamin B12, Vitamin D, Vitamin E, L-Taurine, and Potassium. In a further aspect, each of the foregoing cargo entities can be encapsulated in a separate nanoparticle carrier and combined together in a powdered composition suitable for incorporation into a food or beverage product.

In one aspect, the composition comprises the following cargo entities, each of which can be encapsulated by the same or a different carrier: Bilberry extract, Vitamin B3 (Niacin), Vitamin B12, Vitamin E, Coenzyme Q10, Krill oil, L-Carnitine, L-Taurine, Magnesium Oxide, Naringin Extract, Pomegranate Extract, and Hawthorn Berry Extract. In a further aspect, each of the foregoing cargo entities can be encapsulated in a separate nanoparticle carrier and combined together in a powdered composition suitable for incorporation into a food or beverage product.

In one aspect, the composition comprises the following cargo entities, each of which can be encapsulated by the same or a different carrier: Fish Oil, Zinc, Vitamin A, Vitamin B5 (Pantothenic acid), Vitamin E, and Vitamin D. In a further aspect, each of the foregoing cargo entities can be encapsulated in a separate nanoparticle carrier and combined together in a powdered composition suitable for incorporation into a food or beverage product.

In one aspect, the composition comprises the following cargo entities, each of which can be encapsulated by the same or a different carrier: Dong Quai Root, Iron, Fish Oil, Vitamin B1 (Thiamin), Vitamin B2 (Riboflavin), Vitamin B6, Vitamin B9 (Folic Acid), Vitamin B12, Aluminum hydroxide, Nettle root extract, Astragalus root powder, and Choline. In a further aspect, each of the foregoing cargo entities can be encapsulated in a separate nanoparticle carrier and combined together in a powdered composition suitable for incorporation into a food or beverage product.

### II. Acceptable Salts of Cargo Entities and Carriers

In some aspects, a naturally-occurring cargo entity or sugar present in the dispersion or a product prepared therefrom can be present as an acceptable, non-naturally occurring salt. Thus, a naturally occurring cargo entity or sugar present in the composition can be present as a non-naturally occurring acid or base salt of the naturally occurring cargo entity or sugar. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, and quaternary ammonium (methyl iodide, ethyl iodide, and the like) salts.

Acceptable salts can be prepared by reaction of the cargo entity or sugar with a mineral or organic acid or an inorganic base, such as salts including sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, isobutyrates, caproates, heptanoates, propiolates, oxalates, malonates, succinates, suberates, sebacates, fumarates, maleates, butyne-1,4-dioates, hexyne- 1 ,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, β-hydroxybutyrates, glycolates, tartrates, methane-sulfonates, propanesulfonates, naphthalene- 1 -sulfonates, naphthalene-2-sulfonates, and mandelates.

According to one aspect, if the cargo entity or sugar has one or more acidic functional groups, the desired salt can be prepared by any suitable method known in the art, for example, treatment of the free acid with an inorganic or organic base, such as an amine (primary, secondary or tertiary), an alkali metal hydroxide or alkaline earth metal hydroxide, or the like. It is understood that the acceptable salts are non-toxic and suitable for ingestion. Additional information on suitable acceptable salts can be found in Remington’s Pharmaceutical Sciences, 17th ed., Mack Publishing Company, Easton, Pa., 1985, which is incorporated herein by reference.

In some aspects, a product prepared from a disclosed dispersion can comprise a naturally-occurring cargo entity and/or sugar present along with an acceptable, non-naturally occurring carrier. Various suitable non-naturally occurring carriers are described in Remington’s Pharmaceutical Sciences, 17th ed., Mack Publishing Company, Easton, Pa., 1985, which is incorporated herein by reference. Non-limiting examples include non-naturally occurring polymeric carriers or binders in liquid or solid form, such as polyglycolic acids, synthetic polymers, non-naturally occurring conjugates of proteins, and the like.

### III. Exemplary Cannabinoid Dispersions

Specific, non-limiting examples of dispersions useful for preparing the cannabinoid nanoparticle include those listed in Table 1. The compositions listed in Table 1 include dispersions comprising a sugar, sugar alcohol, or sugar substitute selected from a sugar comprising sucrose, fructose, and glucose (e.g., cane sugar or organic cane sugar); a sugar alcohol selected from erythritol or xylitol; maltodextrin, or sucralose. In one aspect, the exemplary dispersion compositions listed in Table 1 include a sugar comprising sucrose, fructose, and glucose such as cane sugar or organic cane sugar. In various aspects, the CBD listed in Table 1 can be CBD isolate, e.g., CBD isolate comprising at least about 99% CBD. The cannabinoids listed in Table 1 can be present in the composition alone or in any combination.

TABLE 1

Exemplary Cannabinoid Dispersion Compositions

| Cannabinoid | Mass Ratio of Cargo Entity to Sugar / Sugar Alcohol / Sugar Substitute | Mass Ratio of Sugar / Sugar Alcohol / Sugar Substitute to Water |
|---|---|---|
| CBD, CBDP, CBG, THC, THCP, or CBN | 1:300 to 1:3 | 1:8 to 1:1.2 |
| CBD, CBDP, CBG, THC, THCP, or CBN | 1:300 to 1:3 | 1:8 to 1:1.3 |
| CBD, CBDP, CBG, THC, THCP, or CBN | 1:300 to 1:3 | 1:8 to 1:1.5 |
| CBD, CBDP, CBG, THC, THCP, or CBN | 1:300 to 1:3 | 1:6 to 1:4 |
| CBD, CBDP, CBG, THC, THCP, or CBN | 1:300 to 1:3 | 1:4 |
| CBD, CBDP, CBG, THC, THCP, or CBN | 1:50 to 1:3 | 1:8 to 1:1.2 |
| CBD, CBDP, CBG, THC, THCP, or CBN | 1:50 to 1:10 | 1:8 to 1:1.2 |
| CBD, CBDP, CBG, THC, THCP, or CBN | 1:40 to 1:10 | 1:8 to 1:1.2 |
| CBD, CBDP, CBG, THC, THCP, or CBN | 1:30 to 1:10 | 1:8 to 1:1.2 |
| CBD, CBDP, CBG, THC, THCP, or CBN | 1:30 to 1:10 | 1:8 to 1:1.2 |
| CBD, CBDP, CBG, THC, THCP, or CBN | 1:30 to 1:15 | 1:8 to 1:1.2 |
| CBD, CBDP, CBG, THC, THCP, or CBN | 1:20 | 1:8 to 1:1.2 |
| CBD, CBDP, CBG, THC, THCP, or CBN | 1:50 to 1:10 | 1:8 to 1:1.3 |
| CBD, CBDP, CBG, THC, THCP, or CBN | 1:50 to 1:10 | 1:8 to 1:1.5 |
| CBD, CBDP, CBG, THC, THCP, or CBN | 1:50 to 1:10 | 1:6 to 1:4 |
| CBD, CBDP, CBG, THC, THCP, or CBN | 1:50 to 1:10 | 1:4 |
| CBD, CBDP, CBG, THC, THCP, or CBN | 1:30 to 1:10 | 1:8 to 1:1.3 |
| CBD, CBDP, CBG, THC, THCP, or CBN | 1:30 to 1:10 | 1:8 to 1:1.5 |
| CBD, CBDP, CBG, THC, THCP, or CBN | 1:30 to 1:10 | 1:6 to 1:4 |
| CBD, CBDP, CBG, THC, THCP, or CBN | 1:30 to 1:10 | 1:4 |
| CBD, CBDP, CBG, THC, THCP, or CBN | 1:30 to 1:15 | 1:8 to 1:1.3 |
| CBD, CBDP, CBG, THC, THCP, or CBN | 1:30 to 1:15 | 1:8 to 1:1.5 |
| CBD, CBDP, CBG, THC, THCP, or CBN | 1:30 to 1:15 | 1:6 to 1:4 |
| CBD, CBDP, CBG, THC, THCP, or CBN | 1:30 to 1:15 | 1:4 |
| CBD, CBDP, CBG, THC, THCP, or CBN | 1:20 | 1:8 to 1:1.3 |
| CBD, CBDP, CBG, THC, THCP, or CBN | 1:20 | 1:8 to 1:1.5 |
| CBD, CBDP, CBG, THC, THCP, or CBN | 1:20 | 1:6 to 1:4 |
| CBD, CBDP, CBG, THC, THCP, or CBN | 1:20 | 1:4 |

In some aspects, the cannabinoid nanoparticles have a particle size that is much lower than existing cannabinoid delivery systems, enabling better bioabsorption among other beneficial properties. In some aspects, the disclosed nanoparticles have a size ranging from about 200 nm to about 2,000 nm, e.g., from about 200 nm to about 1,000 nm, or from about 200 nm to about 500 nm. In a further aspect, the disclosed nanoparticles have a size ranging from about 1 nm to about 1,000 nm, e.g. from about 1 nm to about 200 nm, or from about 1 nm to about 50 nm. Particle size can be determined using methods known in the art, e.g., light scattering or zeta potential measurements. Particle size, as referred to herein, refers to the mean or average particle size of a given cannabinoid nanoparticle sample. The inventors have surprisingly discovered that the disclosed method enables the production of cannabinoid nanoparticles that are 1,000 times smaller than other existing cannabinoid delivery systems. Any of the cannabinoid nanoparticles described above can be combined with one or more nanoparticle-encapsulated cargo entities that comprise a non-cannabinoid cargo entity.

### D. Products Comprising the Nanoparticles

Also described herein are products comprising the nanoparticle prepared by a disclosed method. One advantage of the disclosed method is that it allows for the infusion of a cargo entity into a comestible product that masks the taste of the cargo entity. In one aspect, for example, a water-based or alcohol-based comestible can comprise the nanoparticle-encapsulated cargo entity or combination of nanoparticle-encapsulated cargo entities prepared by a disclosed method, together with a water-based or alcohol-based liquid. In another aspect, the nanoparticle-encapsulated cargo entity can be at least partially dissolved in the comestible liquid, e.g., water-based or alcohol-based liquid. In a further aspect, the comestible liquid such as a water-based or alcohol-based liquid can comprise the nanoparticle-encapsulated cargo entity uniformly dispersed therein. In a further aspect, a solid or semi-solid comestible can comprise the nanoparticle-encapsulated cargo entity prepared by a disclosed method, together with one or more foodstuff ingredients.

In various aspects, the nanoparticle-encapsulated cargo entity prepared by a disclosed method can be infused into a water-based or alcohol-based comestible by mixing the nanoparticle-encapsulated cargo entity into the liquid, e.g., by stirring or agitating the mixture until no solid nanoparticle-encapsulated cargo entity particles are observed. The nanoparticle-encapsulated cargo entity can be infused into a variety of beverages, including without limitation flavored or unflavored carbonated or still water, beer, wine, hard seltzer, cocktails, spirits, sports beverages, beauty water, and the like.

### 1. Solid and Semi-Solid Comestibles

In further aspects, the nanoparticle-encapsulated cargo entity prepared by a disclosed method can be infused into a solid or semi-solid comestible. Examples of suitable comestibles include without limitation vitamins, supplements, concentrates, extracts, capsules, tablets, powders, lozenges, chewing gums, chewable candies, hard candies, cakes, chocolate bars, granola bars, nut bars, and the like. The comestible food product can comprise one or more foodstuff ingredients, including without limitation starch, sugar, sugar alcohols, nuts, eggs, milk, chocolate powder, cream, water, emulsifiers, food preservatives, and other ingredients common in food. In a further aspect, the cargo entity nanoparticles prepared by a disclosed method can be incorporated into various comestible sex aids, including without limitation creams, gels, oils, and the like.

In other aspects, the nanoparticle-encapsulated cargo entity prepared by a disclosed method can be incorporated into a comestible product suitable for animal or pet, such as a dog. Examples include without limitation flavored pet or dog treats, chews, bones, biscuits, cookies, jerkies, and the like. Without wishing to be bound by theory, it is believed that the cargo entity nanoparticles prepared by a disclosed method can be useful for helping with an animal or pet’s anxiety, stress, pain, inflammation, arthritis, seizures, digestion problems, among others.

### 2. Products for Therapeutic Uses

In further aspects, the nanoparticle-encapsulated cargo entity prepared by a disclosed method can be incorporated into a comestible product for therapeutic use, including products suitable for humans and animals. Without wishing to be bound by theory, the nanoparticle-encapsulated cargo entity can be useful for treating a variety of conditions, disorders, and illnesses. Examples include without limitation lack of appetite, stress, anxiety, depression, nausea, motion sickness, vomiting, weight loss, inflammation, arthritis, gout, epilepsy, addiction, drug or alcohol dependence, pain, attention deficit disorder (ADD), autism/Asperger’s disorder, psychiatric disorders including bipolar disorder, mania, obsessive compulsive disorder (OCD), insomnia, digestive disorders, among others.

In various aspects, the nanoparticle-encapsulated cargo entity prepared by a disclosed method can be formulated as a composition or formulation comprising a suitable carrier. Non-limiting examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and organic esters such as ethyl oleate. Proper fluidity can be maintained, for example, by the use of coating materials such as lecithin, by the maintenance of the required particle size in the case of dispersions and by the use of surfactants.

Suitable carriers for a product for therapeutic use can also comprise adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Suitable inert carriers can include sugars such as lactose.

In some aspects, the product for therapeutic use can include an excipient. Suitable excipients include, without limitation, saccharides, for example, glucose, lactose, or sucrose, mannitol, or sorbitol, cellulose derivatives, and/or calcium phosphate, for example, tricalcium phosphate or acidic calcium phosphate.

In further aspects, the product for a therapeutic use can include a binder. Suitable binders include, without limitation, tare compounds such as starch paste, for example, corn, wheat, rice, and potato starch, gelatin, tragacanth, methylcellulose, hydroxypropyl methylcellulose, carboxymethylcellulose, and/or polyvinylpyrrolidone. In still further aspects, there can be a disintegrating agent, such as the aforementioned starches and carboxymethyl starch, crosslinked polyvinylpyrrolidone, agar, or alginic acid or a salt thereof, such as sodium alginate.

In some aspects, the product for a therapeutic use can include an additive. Examples of additives include, but are not limited to, diluents, buffers, binders, surface-active agents, lubricants, humectants, pH adjusting agents, preservatives (including anti-oxidants), emulsifiers, occlusive agents, opacifiers, antioxidants, colorants, flavoring agents, gelling agents, thickening agents, stabilizers, and surfactants, among others. Thus, in various further aspects, the additive is vitamin E, gum acacia, citric acid, stevia extract powder, Luo Han Gou, Monoammonium Glycyrrhizinate, Ammonium Glycyrrhizinate, honey, or combinations thereof. In a still further aspect, the additive is a flavoring agent, a binder, a disintegrant, a bulking agent, or silica. In a further aspect, the additive can include flowability-control agents and lubricants, such as silicon dioxide, talc, stearic acid and salts thereof, such as magnesium stearate or calcium stearate, and/or propylene glycol.

The therapeutic product can be formulated for oral use, such as for example, a tablet, pill, or capsule, and the composition can include a coating layer that is resistant to gastric acid. Such a layer, in various aspects, can include a concentrated solution of saccharides that can comprise gum arabic, talc, polyvinylpyrrolidone, polyethylene glycol, and/or titanium dioxide, and suitable organic solvents or salts thereof.

The effective amount of the cargo entity in the therapeutic product can vary within wide limits. Such a dosage can be adjusted to the individual requirements in each particular case including the specific composition(s) being administered and the condition being treated, as well as the subject being treated. In general, single dose compositions can contain such amounts or submultiples thereof of the composition to make up the daily dose. The dosage can be adjusted in the event of any contraindications. Dosage can vary, and can be administered in one or more dose administrations daily, for one or several days.

### E. Methods for Treating a Disorder

In one aspect, disclosed is a method of treating a condition in a subject, comprising administering to the subject a disclosed nanoparticle-encapsulated cargo entity composition. In a further aspect, disclosed is a method of treating a condition in a subject, comprising administering to the subject the product of a disclosed method, a disclosed water-based or alcohol-based comestible, or a disclosed solid or semi-solid comestible.

According to one aspect, the subject is a mammal. In a further aspect, the subject is a human. In a still further aspect, the subject has been diagnosed with a need for treatment of the disorder prior to the administering step. In a further aspect, the subject is at risk for developing the disorder prior to the administering step. In a further aspect, the method further comprises the step of identifying a subject in need of treatment of the disorder.

In various aspects, the disorder is lack of appetite, stress, anxiety, depression, nausea, motion sickness, vomiting, weight loss, inflammation, arthritis, gout, epilepsy, addiction, drug or alcohol dependence, pain, attention deficit disorder (ADD), autism/Asperger’s disorder, a psychiatric disorder, bipolar disorder, mania, obsessive-compulsive disorder (OCD), insomnia, or a digestive disorder.

The product administered to the subject can be formulated for oral use, such as for example, a tablet, pill, or capsule, and the composition can include a coating layer that is resistant to gastric acid, as described above. Such a layer, in various aspects, can include a concentrated solution of saccharides that can comprise gum arabic, talc, polyvinylpyrrolidone (PVP), polyethylene glycol, and/or titanium dioxide, and suitable organic solvents or salts thereof. In a further aspect, the product can be administered to the subject as a nasal spray, inhaler, tincture, topically applied as a cream, or via syringe or dropper.

The effective amount of the cargo entities in the product administered to the subject can vary within wide limits. Such a dosage can be adjusted to the individual requirements in each particular case including the specific composition(s) being administered and the condition being treated, as well as the subject being treated. In general, single dose compositions can contain such amounts or submultiples thereof of the composition to make up the daily dose. The dosage can be adjusted in the event of any contraindications. Dosage can vary, and can be administered in one or more dose administrations daily, for one or several days.

### F. Examples

The following examples are put forth so as to provide those of ordinary skill in the art with a complete disclosure and description of how the methods and products claimed herein are made and evaluated, and are intended to be purely exemplary and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in °F or is at ambient temperature, and pressure is at or near atmospheric. The Examples are provided herein to illustrate the invention, and should not be construed as limiting the invention in any way.

To prepare the exemplary dispersions, an induction cooktop was used to maintain consistent heat, along with a stainless steel pot as a vessel for the dispersion, and stainless steel or glass utensils for stirring or agitation. The desired amount of organic cane sugar and filtered or spring water was first mixed before the addition of the cargo entity. A desired amount of cargo entity was then added to the mixture, and the induction cooktop was set at a temperature range of from 212° F. to 280° F. It was observed that setting the temperature of the induction cooktop to a temperature above 280° F. can affect the quality of the nanoparticle-encapsulated cargo entity recovered from the dispersion. The dispersion was then brought to a boil. After about ¾ of the volume of the original dispersion boiled away, the dispersion was agitated with stirring while keeping the dispersion heated on the induction cooktop. The dispersion was stirred and agitated but not so vigorously as to create a vortex in the dispersion.

Optionally, depending on the ratio of organic cane sugar to the water, an optional seeding step was performed. Incrementally, a pinch (about 0.36 grams) of organic can sugar was added to the dispersion with stirring until visible solidification (e.g., precipitation) of the nanoparticle-encapsulated cargo entity was observed. The sides of the stainless steel vessel were also scraped to dislodge solidified nanoparticle-encapsulated cargo entity and to aid in further solidification of the nanoparticle from the dispersion.

The stainless steel vessel comprising the nanoparticle-encapsulated cargo entity and remaining liquid was then removed from the induction cooktop when most of the liquid had evaporated from the dispersion and the dispersion resembled wet sand. The dispersion was continuously stirred until the remaining liquid evaporated, which yielded granulated solid nanoparticle-encapsulated cargo entity particles resembling dry sand.

The nanoparticle-encapsulated cargo entity recovered from the dispersion was then ground into a fine powder using a food processor. The ground nanoparticle was stored in a dry, dark and cool location (less than about 75° F.) until further use.

Using a stir bar or other similar device, the nanoparticle-encapsulated cargo entity was infused into the desired food or beverage comestible. When a tabletop stir bar or other similar device is used, low to medium speed was sufficient to ensure that all cargo entity nanoparticle particles were infused into the food or beverage. Infusion was complete when no solid nanoparticle-encapsulated cargo entity particles were observed in the food or beverage.

The following tables list exemplary compositions in comprising cargo-entity-encapsulated nanoparticles. Although certain amounts of cargo entities are listed, it is contemplated that any suitable amount of the stated ingredient may be used. Any of the formulations listed in the tables below can be formulated as a dry powder or alternatively added to a liquid, beverage, food, or other comestible product such as a protein bar and the like.

Table 2 lists exemplary cannabinoid nanoparticles recovered from dispersions prepared according to the preceding exemplary method.

TABLE 2

Example Dispersions/Cannabinoid Nanoparticles

| Sugar/Sugar Alcohol/Sugar Substitute (g) | Cannabinoid (g) | Water (mL) | Mass Ratio of Cannabinoid to Sugar / Sugar Alcohol / Sugar Substitute | Mass Ratio of Sugar / Sugar Alcohol / Sugar Substitute to Water |
|---|---|---|---|---|
| Organic Cane Sugar (222.73) | 2-12 (CBD) | 901.01 | 1:111.3 to 1:18.6 | about 1:4 |
| Organic Cane Sugar (55.19) | 0.5 (CBD) | 225.38 | 1:110.4 | about 1:4 |
| Erythritol (55.19)* | 0.5 (CBD) | 225.3 | 1:110.4 | about 1:4 |
| Truvia (Erythritol, Sugar, Stevia Leaf Extract) (55.19) | 1.0 (CBD) | 225.3 | 1:55.2 | about 1:4 |
| Organic Stevia Extract (35.07) | 0.5 (CBD) | 142.9 | 1:70.1 | about 1:4 |
| Liquid Organic Sugar-Free Stevia (Deionized Water, Organic Stevol Glycosides from Stevia Leaf, 11% Organic Alcohol) (55.19) | 0.5 (CBD) | 225.3 | 1:110.4 | about 1:4 |
| Swerve (Erythritol, Oligosaccharides, Natural Flavors) (55.19) | 1.0 (CBD) | 225.3 | 1:55.2 | about 1:4 |
| Sucralose/Maltodextrin* (55.19) | 0.5 (CBD) | 225.3 | 1:110.4 | about 1:4 |
| Sucralose/Maltodextrin (55.19) | 1.0 (CBD) | 225.3 | 1:55.2 | about 1:4 |
| Monk Fruit Sweetener with Erythritol (55.19) | 0.5 (CBD) | 225.3 | 1:110.4 | about 1:4 |
| Superlose (Granular Allulose, Monk Fruit Extract, Stevia Extract, Natural Flavors) (55.19) | 1.0 (CBD) | 225.3 | 1:55.2 | about 1:4 |
| Organic Cane Sugar (75.19) | 10 (Δ⁸-THC) | 225.3 | 1:7.5 | about 1:4 |

*dispersion was cooled and seeded before solidification of cannabinoid nanoparticle was observed

It is contemplated that the cannabinoid nanoparticles recovered from the dispersions listed in Table 1 can be combined with other nanoparticle-encapsulated cargo entities. Suitable cargo entites include for example nutraceuticals, active pharmaceutical ingredients (APIs), minerals, inorganic or organic salts, supplements, amino acids, peptides, polypeptides, plant extracts, ubiquinones, herbs, vitamins, or any combination thereof.

Table 3 lists a dry powder nanoparticle-encapsulated cargo entity formulation wherein each cargo entity is encapsulated by a cane sugar carrier to form a nanoparticle-encapsulated cargo entity. Each nanoparticle-encapsulated cargo entity is then combined to form a composition having a plurality of nanoparticle-encapsulated cargo entities. The combined composition can optionally be ground into a fine powder and added to 16 or 32 ounces of a fluid such as water to form a beverage. The composition listed in Table 3 is formulated to be NCAA compliant. The ratio of nanoparticle to cargo entity is 6.5:1 unless indicated otherwise. The combined composition can optionally be ground into a fine powder and added to 16 or 32 ounces of a fluid such as water to form a beverage.

TABLE 3

Powder Formulation for NCAA-Compliant Beverage

| Cargo Entity | Total weight of nanoparticles (deviation from 6.5:1 nanoparticle:cargo ratio) | Cargo Entity Amount | Wt.% Cargo Entity by Total Nanoparticle Weight |
|---|---|---|---|
| Beet Root (Organic) | 1.3 g | 200 mg | 15.4 |
| Ginger Root | 0.975 g | 150 mg | 15.4 |
| Ceylon Cinnamon | 1.3 g | 150 mg | 11.5 |
| Beta Carotene | 0.065 g | 10 mg | 15.4 |
| Thiamine (Vitamin B1) | 0.15 g (50:1 ratio) | 3mg | 2.0 |
| Riboflavin (Vitamin B2) | 0.05 g (100:1 ratio) | 0.5mg | 1.0 |
| Pantothenic Acid (Vitamin B5) | 0.65 g | 100 mg | 15.4 |
| Pyridoxine (Vitamin B6) | 0.05 g (100:1 ratio) | 0.5mg | 1.0 |
| Biotin (Vitamin B7) | 0.05 g (50:1 ratio) | 1ms | 2.0 |
| Folate or Folic Acid (Vitamin B9) | 0.04 g (100:1 ratio) | 0.4mg | 1.0 |
| Cobalamin (Vitamin B12) | 0.05 g (50:1 ratio) | 1mg | 2.0 |
| Vitamin D3 | 0.05 g (1000:1 ratio) | 0.05 mg | 0.1 |
| Vitamin E | 0.162 g | 25 mg | 15.4 |
| Sodium Bicarbonate | 1.3 g | 200 mg | 15.4 |
| Selenium | 0.04 g (100:1 ratio) | 0.4mg | 1.0 |
| Zinc (Zinc Oxide) | 1.3 g | 10 mg | 0.8 |
| Calcium Lactate | 1.3 g | 200 mg | 15.4 |
| Iron (Ferrous Fumarate) | 0.325 g | 50 mg | 15.4 |
| Magnesium Oxide | 0.78 g | 120 mg | 15.4 |
| Potassium Citrate | 1.3 g | 200 mg | 15.4 |

Table 4 lists a dry powder nanoparticle-encapsulated cargo entity formulation wherein each cargo entity is encapsulated by a cane sugar carrier to form a nanoparticle-encapsulated cargo entity. Each nanoparticle-encapsulated cargo entity is then combined to form a composition having a plurality of nanoparticle-encapsulated cargo entities. The combined composition can optionally be ground into a fine powder and added to 16 or 32 ounces of a fluid such as water to form a beverage. The ratio of nanoparticle to cargo entity is 6.5:1 unless indicated otherwise. The combined composition can optionally be ground into a fine powder and added to 16 or 32 ounces of a fluid such as water to form a beverage.

TABLE 4

Powder Formulation for Beverage for Elite Athletes

| Cargo Entity | Total weight of nanoparticles (deviation from 6.5:1 nanoparticle:cargo ratio) | Cargo Entity Amount | Wt.% Cargo Entity by Total Nanoparticle Weight |
|---|---|---|---|
| Asian Ginseng | 0.975 g | 150 mg | 15.4 |
| Beet Root | 1.3 g | 200 mg | 15.4 |
| Caralluma Fimbriata | 1.3 g | 200 mg | 15.4 |
| Cannabidiol (CBD) | 0.0325 g | 5 mg | 15.4 |
| Ceylon Cinnamon | 0.975 g | 150 mg | 15.4 |
| Conjugated linoleic acid (CLA) | 1.3 g | 200 mg | 15.4 |
| Coenzyme Q10 (CoQ10) | 0.65 g | 100 mg | 15.4 |
| Curcumin | 0.65 g | 100 mg | 15.4 |
| Garcinia Cambogia | 0.65 g | 100 mg | 15.4 |
| Ginger Root | 0.975 g | 150 mg | 15.4 |
| Guarana | 1.3 g | 200 mg | 15.4 |
| Ashwagandha | 0.975 g | 150 mg | 15.4 |
| L-Arginine | 0.65 g | 100 mg | 15.4 |
| L-Carnitine | 0.65 g | 100 mg | 15.4 |
| L-Theanine | 0.65 g | 100 mg | 15.4 |
| Magnesium Oxide | 0.78 g | 120 mg | 15.4 |
| Moringa | 1.3 g | 200 mg | 15.4 |
| Methylsulfonylmethane (MSM) | 1.3 g | 200 mg | 15.4 |
| Turmeric | 0.65 g | 100 mg | 15.4 |
| Beta Hydroxy Beta-Methylbutyrate | 1.3 g | 200 mg | 15.4 |
| Peppermint oil | 1.625 g | 250 mg | 15.4 |
| Vitamin D3 | 0.05 g (1000:1 ratio) | 0.05 mg | 0.1 |
| Beta Alanine | 1.3 g | 200 mg | 15.4 |

The following example is directed to a pharmaceutical product. The compositions and methods described above can be used to process pharmaceutical compounds in order to individualize a patient’s daily regiment into a powder formulation. This customized formulation can be consumed as it is or added to a beverage and drunk by the patient, avoiding worry that typically comes with keeping track of multiple tablets and capsules, choking hazards, and cumbersome prescription bottles. This approach would favor patients that have difficulty swallowing pills or are limited to certain medications due to a feeding tube. Nanoparticle complex powders could be utilized for in patient or outpatient settings, nursing homes, LTCF, FEMA, EMS, and even military especially when in austere environments.

The following is a recipe based on the process described above, using 10 g of API and 55 g of sugar (yielding a final 65 g of nanoparticle-encapsulated cargo entity). Hypothetical patient is currently on a daily regimen of: Lisinopril 20 mg daily; Hydrochlorothiazide 25 mg daily; Atenolol 50 mg daily; Irbesartan 150 mg twice daily; Citalopram 20 mg daily. This patient would receive 2 single serving packets labeled according to regulatory guidelines. The morning packet will contain the following nanoparticle complex powder amounts equaling the above patient prescription strengths and can be mixed in water for the patient to consume: nanoparticle/Lisinopril 130 mg, nanoparticle/ Hydrochlorothiazide 162.5 mg, nanoparticle/Atenolol 325 mg, nanoparticle/Irbesartan 975 mg, nanoparticle/Citalopram 130 mg. Each of the nanoparticle compositions would be combined together in powder form. The second packet for later in the day will contain nanoparticle/Irbesartan 975 mg. Details of the formulations are shown in Table 5.

TABLE 5

API Formulation

| | | |
|---|---|---|
| API | Total wt. nanoparticle (6.5:1 nanoparticle:API ratio) | API Amount |
| Lisinopril | 130 mg | 20 mg |
| Hydrochlorothiazide | 162.5 mg | 25 mg |
| Atenolol | 325 mg | 50 mg |
| Irbesartan | 975 mg | 150 mg |
| Citalopram | 130 mg | 20 mg |

The following example is a mixture utilizing an API, vitamin, and nutraceutical combined with a flavoring agent to mask unsavory tastes of the API.

TABLE 6

API Formulation

| | | |
|---|---|---|
| API/Cargo | Total wt. nanoparticle (deviation from 6.5:1 nanoparticle:API ratio) | API/Cargo Amount |
| Ethyl estradiol | 0.05 g (100:1 ratio) | 0.5 mg |
| Vitamin D3 | 0.05 g (1000:1 ratio) | 0.05 mg |
| Pyroxidine (Vit B6) | 2.5 g (100:1 ratio) | 25 mg |
| Lime Doterra oil | 1.0 g | N/A |

The following is another example of a pharmaceutical formulation.

TABLE 7

API Formulation

| | | |
|---|---|---|
| API/Cargo | Total wt. nanoparticle (6.5:1 nanoparticle:API ratio) | API/Cargo Amount |
| Erythromycin | 3.25 g | 500 mg |
| Guaifenesin | 2.6 g | 400 mg |
| Echinacea | 1.625 g | 250 mg |
| Turmeric | 1.625 g | 250 mg |
| Prednisone | 0.13 g | 20 mg, |

The following is an example of a nanoparticle formulation that can be added to a hard seltzer as a base.

TABLE 8

Formulation for Hard Seltzer

| | | |
|---|---|---|
| Cargo | Total wt. nanoparticle (deviation from 6.5:1 nanoparticle:cargo ratio) | Cargo Amount |
| Moringa | 1.3 g | 200 mg |
| CBD | 0.195 g | 30 mg |
| Choline Bitartrate | 0.325 g | 50 mg |
| Folic Acid | 0.04 g (100:1 ratio) | 0.4 mg |

The following is an example of a nanoparticle formulation that can be added to a wine as a base.

TABLE 9

Formulation for Wine

| | | |
|---|---|---|
| Cargo | Total wt. nanoparticle (deviation from 6.5:1 nanoparticle:cargo ratio) | Cargo Amount |
| Maca | 0.65 g | 100 mg |
| Ceylon Cinnamon | 0.325 g | 50 mg |
| Clove Oil | 1.0 g | n/a |
| Folic Acid | 0.04 g(100:1 ratio) | 0.4 mg |
| Vitamin C | 3.25 g | 500 mg |

The following is an example of a nanoparticle formulation that can be added to a cider as a base.

TABLE 10

Formulation for Cider

| | | |
|---|---|---|
| Cargo | Total wt. nanoparticle (6.5:1 nanoparticle:cargo ratio) | Cargo Amount |
| Ceylon Cinnamon | 0.325 g | 50 mg |
| KSM-66 | 0.975 g | 150 mg |
| Tumeric | 1.3 g | 200 mg |
| Magnesium Oxide | 0.325 g | 50 mg |

The following is an example of a nanoparticle formulation that can be added to a beer as a base.

TABLE 11

Formulation for Beer

| | | |
|---|---|---|
| Cargo | Total wt. nanoparticle (deviation from 6.5:1 nanoparticle:cargo ratio) | Cargo Amount |
| Folic Acid | 0.04 g (100:1 ratio) | 0.4 mg |
| L-Theanine | 0.65 g | 100 mg |
| Asian Ginseng | 0.162 g | 25 mg |
| Nanoparticle Encapsulated Hop Oil | 0.75 g | N/A |

The following is an example of a nanoparticle formulation that can be added to a distilled spirit as a base.

TABLE 12

Formulation for Distilled Spirits

| | | |
|---|---|---|
| Cargo | Total wt. nanoparticle (deviation from 6.5:1 nanoparticle:cargo ratio) | Cargo Amount |
| Caralluma Fimbriata | 1.3 g | 200 mg |
| Milk Thistle | 1.95 g | 300 mg |
| Beet Root | 1.3 g | 200 mg |
| Selenium | 0.04 g (100:1 ratio) | 0.4 mg |
| Nanoparticle-encapsulated bacon grease | 1.0 g | N/A |

The following is an example of a nanoparticle formulation suitable for administration to a pet such as a dog (e.g., a senior dog) as an infused water.

TABLE 13

Formulation for Pet Water

| | | |
|---|---|---|
| Cargo | Total wt. nanoparticle (deviation from 6.5:1 nanoparticle:cargo ratio) | Cargo Amount |
| Turmeric | 325 mg | 50 mg |
| Flaxseed Oil | 6.5 g | 1 g |
| Glucosamine | 650 mg | 100 mg |
| Yucca Root | 6.5 g | 1 g |
| Kelp Seaweed | 4.55 g | 0.7 g |
| Quercetin | 162 mg | 25 mg |
| Coenzyme Q10 | 500 mg (100:1 ratio) | 5 mg |
| Lipoic Acid | 100 mg (100:1 ratio) | 1 mg |

The following is an example of a nanoparticle formulation suitable for administration to a pet such as a dog (e.g., a dog weighing about 10 lbs.) as an infused water.

TABLE 14A

Formulation for Pet Water

| | | |
|---|---|---|
| Cargo | Total wt. nanoparticle (deviation from 6.5:1 nanoparticle:cargo ratio) | Cargo Amount |
| Tree Barks Powder | 9.75 g | 1.5 g |
| Psyllium | 6.5 g | 1.0 g |
| Ginger Root Extract | 325 mg | 50 mg |

The following is an example of a nanoparticle formulation suitable for a human as a beauty water.

TABLE 14B

Formula beauty water (for humans)

| | | |
|---|---|---|
| Cargo | Total wt. nanoparticle (deviation 6.5:1 nanoparticle:cargo ratio) | Cargo Amount |
| Collagen | 65 mg | 10 mg |
| Biotin | 75 mg (5000:1 ratio) | 0.015 mg |
| Vitamin E | 300 mg (100:1 ratio) | 5 mg |
| Vitamin D3 | 325 mg | 50 mg |
| Folate | 50 mg (100:1 ratio) | 0.5 mg |
| Beta Carotene | 250 mg (50:1 ratio) | 5 mg |

The following is an example of a nanoparticle formulation suitable for as an after-party beverage.

TABLE 14C

Formula for After-Party Drink

| | | |
|---|---|---|
| Cargo | Total wt. nanoparticle (deviation 6.5:1 nanoparticle:API ratio) | Cargo Amount |
| Betaine | 6.5 g | 1 g |
| Thiamine (Vitamin B1) | 0.15 g (50:1 ratio) | 3 mg |
| Riboflavin (Vitamin B2) | 0.05 g (100:1 ratio) | 0.5 mg |
| Pantothenic Acid (Vitamin B5) | 0.65 g | 100 mg |
| Pyridoxine (Vitamin B6) | 0.05 g (100:1 ratio) | 0.5 mg |
| Biotin (Vitamin B7) | 0.05 g (50:1 ratio) | 1 mg |
| Folate or Folic Acid (Vitamin B9) | 0.04 g (100:1 ratio) | 0.4 mg |
| Cobalamin (Vitamin B12) | 0.05 g (50:1 ratio) | 1 mg |
| Choline | 975 mg | 150 mg |
| Turmeric | 2.11 g | 325 mg |
| Asian Ginseng | 2.11 g | 325 mg |
| Guarana | 2.11 g | 325 mg |
| Sodium Chloride | 3.25 g | 500 mg |
| Potassium Citrate | 2.11 g | 325 mg |
| Vitamin C | 3.25 g | 500 mg |
| Milk Thistle | 325 mg | 50 mg |
| Glutathione | 650 mg | 100 mg |

The following is an example of a nanoparticle-encapsulated hop oil complex formulation for a beer. This utilizes nano-complexed Hop Oil in place of standard hops pellets or hop oil extract. The complexed Hop Oil will reduce waste of materials including alcohol content, storage and certain temperature constraints, changing flavor profile and will add to the stability and shelf life of the final product.

TABLE 15

Formulation for Hop Oil Beer

| | | | |
|---|---|---|---|
| mount | Ingredient | Type | % or IBU |
| 3.50 lbs. | Amber Liquid Extract (12.5 SRM) | Extract | 46.67% |
| 3.50 lbs. | Pale Liquid Extract (8.0 SRM) | Extract | 46.67% |
| 0.50 lbs. | Caramel Crystal Malt -60 ml(60 SRM) | Grain | 6.67% |
| 292.5 g | Nanoparticle Encapsulated Hop Oil (45 min) | Hops | - |
| 97.5 g | Nanoparticle Encapsulated Hop Oil | Hops | - |
| 1.0 lbs. | Honey (Boil 45 min) | Misc | - |
| 2 packages | Munton Fison Ale (Munton Fison # -) | Yeast Ale | - |

The following is an example of a powdered formulation designed for elite athletes (e.g., for recovery, hydration, etc.). The formulation can be diluted with a suitable amount of water and consumed.

TABLE 16

Formulation for Elite Athletes

| | | | |
|---|---|---|---|
| Active Ingredient | Weight of nanoparticles (deviation from 6.5:1 Complex:API ratio) | API Dosage | Therapeutic Categories |
| Asian Ginseng | 0.975 g | 150 mg | Energy Boost |
| Beet Root | 1.3 g | 200 mg | Antioxidant - Energy Boost |
| Caralluma Fimbriata | 1.3 g | 200 mg | Weight Loss |
| Cannabidiol (CBD) | 0.0325 g | 5 mg | Antioxidant - Antianxiety-Pain Relief |
| Ceylon Cinnamon | 0.975 g | 150 mg | Antioxidant |
| Conjugated linoleic acid (CLA) | 1.3 g | 200 mg | Antioxidant - Weight Loss |
| Coenzyme Q10 (CoQ10) | 0.65 g | 100 mg | Antioxidant - Brain Health -Blood/Heart Health |
| Curcumin | 0.65 g | 100 mg | Antioxidant - Immune Support |
| Garcinia Cambogia | 0.65 g | 100 mg | Weight Loss |
| Ginger Root | 0.975 g | 150 mg | Antioxidant - Digestive Aid |
| Guarana | 1.3 g | 200 mg | Energy Boost |
| Ashwagandha | 0.975 g | 150 mg | Antioxidant - Energy Boost |
| L-Arginine | 0.65 g | 100 mg | Energy Boost |
| L-Carnitine | 0.65 g | 100 mg | Energy Boost - Weight Loss -Blood/Heart Health |
| L-Theanine | 0.65 g | 100 mg | Energy Boost - Antianxiety |
| Sodium Bicarbonate | 1.3 g | 200 mg | Energy Boost -Vitamin/Minerals |
| Magnesium Oxide | 0.78 g | 120 mg | Energy Boost-Blood/Heart Health - Digestive Aid Immune Support |
| Moringa | 1.3 g | 200 mg | Antioxidant |
| Methylsulfonylmethane (MSM) | 1.3 g | 200 mg | Antioxidant - Pain Relief t |
| Turmeric | 0.65 g | 100 mg | Antioxidant - Immune Support |
| Beta Hydroxy Beta-Methylbutyrate | 1.3 g | 200 mg | Energy Boost |
| Peppermint oil | 1.625 g | | Antioxidant - Energy Boost -Digestive Aid |
| Vitamin D | 0.05 g (1000:1 ratio) | 0.05 mg | Antioxidant -Vitamin/Minerals |
| Beta Alanine | 1.3 g | 200 mg | Antioxidant - Energy Boost |

The following is an example of a powdered formulation designed for collegiate athletes (e.g., for recovery, hydration, etc.). The formulation can be diluted with a suitable amount of water and consumed.

TABLE 17

Formulation for Collegiate Athletes

| | | | |
|---|---|---|---|
| Active Ingredient | Weight of nanoparticles (deviation from 6.5:1 Complex:API ratio) | API Dosage | Therapeutic Categories |
| Beet Root | 1.3 g | 200 mg | Antioxidant - Energy Boost |
| Ginger Root | 0.975 g | 150 mg | Antioxidant - Digestive Aid |
| Ceylon Cinnamon | 1.3 g | 150 mg | Antioxidant |
| Beta Carotene | 0.065 g | 10 mg | Antioxidant - Vitamin/Minerals |
| Vitamin B1 (Thiamin) | 0.15 g (50:1 ratio) | 3 mg | Energy Boost - Vitamin/Minerals |
| Vitamin B2 (Riboflavin) | 0.05 g (100:1 ratio) | 0.5 mg | Energy Boost - Vitamin/Minerals |
| Vitamin B5 (Pantothenic acid) | 0.65 g | 100 mg | Energy Boost - Vitamin/Minerals |
| Vitamin B6 | 0.05 g (100:1 ratio) | 0.5 mg | Energy Boost - Vitamin/Minerals |
| Vitamin B7 (Biotin) | 0.05 g (50:1 ratio) | 1 mg | Energy Boost - Vitamin/Minerals |
| Vitamin B9 (Folic Acid) | 0.04 g (100:1 ratio) | 0.4 mg | Energy Boost - Vitamin/Minerals - |
| Vitamin B12 | 0.05 g (100:1 ratio) | 0.5 mg | Energy Boost - Vitamin/Minerals |
| Vitamin D | 0.05 g (1000:1 ratio) | 0.05 mg | Antioxidant- Vitamin/Minerals |
| Vitamin E | 0.162 g | 25 mg | Blood/Heart Health - Antioxidant-Vitamin/Minerals |
| Sodium Bicarbonate | 1.3 g | 200 mg | Energy Boost - Vitamin/Minerals |
| Selenium | 0.04 g (100:1 ratio) | 0.4 mg | Brain Health - Antioxidant -Fur/Skin/Hair Health |
| Zinc | 0.065 g | 10 mg | Energy Boost - Vitamin/Minerals -Immune Support |
| Calcium Lactate | 1.3 g | 200 mg | Vitamin/Minerals |
| Iron | 0.325 g | 50 mg | Energy Boost - Vitamin/Minerals -Immune Support |
| Magnesium Oxide | 0.78 g | 120 mg | Energy Boost-Blood/Heart Health - Digestive Aid Immune Support |
| Potassium Citrate | 3.25 g | 500 mg | Vitamin/Minerals |

The following is an example of a powdered formulation designed for a hydrating recovery drink. The formulation can be diluted with a suitable amount of water and consumed.

TABLE 18

Formulation for Collegiate Athletes

| | | | |
|---|---|---|---|
| Active Ingredient | Weight of nanoparticles (deviation from 6.5:1 Complex:API ratio) | API Dosage | Therapeutic Categories |
| Betaine | 6.5 g | 1 g | Detox/Liver Health |
| L-Taurine | 6.5 g | 1 g | Energy Boost - Brain Health-Blood/Heart Health - Digestive Aid |
| Ginger root extract | 6.5 g | 1 g | Antioxidant - Digestive Aid |
| S-adenosyl-L-methionine | 3.25 g | 500 mg | Detox/Liver Health |
| Thiamine (Vitamin B1) | 0.15 g (50:1 ratio) | 3 mg | Energy Boost - Vitamin/Minerals |
| Riboflavin (Vitamin B2) | 0.05 g (100:1 ratio) | 0.5 mg | Energy Boost - Vitamin/Minerals |
| Vitamin B5 (Pantothenic acid) | 0.65 g | 100 mg | Energy Boost - Vitamin/Minerals |
| Vitamin B6 | 0.05 g (100:1 ratio) | 0.5 mg | Energy Boost - Vitamin/Minerals |
| Vitamin B7 (Biotin) | 0.05 g (50:1 ratio) | 1 mg | Energy Boost - Vitamin/Minerals |
| Vitamin B9 (Folic Acid) | 0.04 g (100:1 ratio) | 0.4 mg | Energy Boost - Vitamin/Minerals - Kidney/Urinary Tract Health |
| Vitamin B12 | 0.05 g (100:1 ratio) | 0.5 mg | Energy Boost - Vitamin/Minerals - Brain Health |
| Choline | 975 mg | 150 mg | Detox/Liver Health -Kidney/Urinary Tract Health |
| Turmeric | 2.11 g | 325 mg | Antioxidant - Immune Support |
| Asian Ginseng | 2.11 g | 325 mg | Energy Boost |
| Guarana | 2.11 g | 325 mg | Energy Boost |
| Sodium Chloride | 3.25 g | 500 mg | Vitamin/Minerals |
| Potassium Citrate | 2.11 g | 325 mg | Vitamin/Minerals |
| Vitamin C | 3.25 g | 500 mg | Antioxidant - Vitamin/Minerals |
| Milk Thistle | 325 mg | 50 mg | Antioxidant - Immune Support |
| Glutathione | 650 mg | 100 mg | Detox/Liver Health |

The following is an example of a powdered formulation designed for a beverage formula designed to improve skin, hair, and/or nails. The formulation can be diluted with a suitable amount of water and consumed.

TABLE 19

Formulation for Skin/Hair/Nails

| | | | |
|---|---|---|---|
| Active Ingredient | Weight of nanoparticles (deviation from 6.5:1 Complex:API ratio) | API Dosage | Therapeutic Categories |
| Vitamin A | 65 mg | 10 mg | Fur/Skin/Hair Health-Vitamin/Minerals - Eye Support |
| Vitamin C | 1.3 g | 200 mg | Antioxidant- Vitamin/Minerals |
| Vitamin E | 325 mg | 50 mg | Blood/Heart Health -Immune Support - Antioxidant- Vitamin/Minerals |
| Coenzyme Q10 | 650 mg | 100 mg | Antioxidant - Brain Health -Blood/Heart Health |
| Vitamin B7 (Biotin) | 100 mg (100:1 ratio) | 1 mg | Fur/Skin/Hair Health - Energy Boost -Vitamin/Minerals |
| Selenium | 40 mg (100:1 ratio) | 0.4 mg | Brain Health - Antioxidant -Fur/Skin/Hair Health |
| Manganese | 65 mg | 10 mg | Vitamin/Minerals |
| Omega-3 | 1.625 g | 250 mg | Fur/Skin/Hair Health -Immune Support - Brain Health - Antioxidant - |
| Omega-6 | 1.625 g | 250 mg | Fur/Skin/Hair Health Immune Support - Brain Health - Antioxidant - |
| Flaxseed Oil | 3.25 g | 500 mg | Antioxidant- Fur/Skin/Hair Health |
| Zinc | 65 mg | 10 mg | Fur/Skin/Hair Health - Energy Boost -Vitamin/Minerals -Immune Support |
| Fern Extract | 1.3 g | 200 mg | Fur/Skin/Hair Health |
| Collagen | 6.75 g (4.5:1 ratio) | 1.5 g | Fur/Skin/Hair Health |
| Keratin | 1.625 g | 250 mg | Fur/Skin/Hair Health - Pain Relief |
| Saw Palmetto | 1.3 g | 200 mg | Fur/Skin/Hair Health |
| Peppermint Oil | 1.625 g | 250 mg | Antioxidant- Fur/Skin/Hair Health |
| Astaxanthin | 65 mg | 10 mg | Antioxidant - Immune Support |
| Iron | 325 mg | 50 mg | Energy Boost - Vitamin/Minerals -Immune Support |

The following is an example of a powdered formulation designed as a women’s multivitamin water. The formulation can be diluted with a suitable amount of water and consumed.

TABLE 20

Formulation for Women’s Multivitamin Water

| | | | |
|---|---|---|---|
| Active Ingredient | Weight of nanoparticles (deviation from 6.5:1 Complex:API ratio) | API Dosage | Therapeutic Categories |
| Reishi Mushroom | 6.5 g | 1 g | Blood/Heart Health |
| Vitamin A | 65 mg | 10 mg | Fur/Skin/Hair Health-Vitamin/Minerals - Eye Support |
| Vitamin B1 (Thiamin) | 300 mg (100:1 ratio) | 3 mg | Energy Boost - Vitamin/Minerals |
| Vitamin B2 (Riboflavin) | 100 mg (100:1 ratio) | 1 mg | Energy Boost - Vitamin/Minerals |
| Vitamin B3 (Niacin) | 195 mg | 30 mg | Energy Boost - Vitamin/Minerals -Brain Health |
| Vitamin B5 (Pantothenic acid) | 650 mg | 100 mg | Energy Boost - Vitamin/Minerals |
| Vitamin B6 | 100 mg (100:1 ratio) | 1 mg | Energy Boost - Vitamin/Minerals |
| Vitamin B9 (Folic Acid) | 50 mg (100:1 ratio) | 0.5 mg | Energy Boost - Vitamin/Minerals -Kidney/Urinary Tract Health |
| Vitamin B12 | 50 mg (100:1 ratio) | 0.5 mg | Energy Boost - Vitamin/Minerals -Brain Health |
| Vitamin C | 1.3 g | 200 mg | Antioxidant- Vitamin/Minerals |
| Vitamin D | 50 mg (1000:1 ratio) | 0.05 mg | Antioxidant- Vitamin/Minerals |
| Vitamin E | 50 mg | 50 mg | Blood/Heart Health -Immune Support - Antioxidant-Vitamin/Minerals |
| Chromium | 70 mg (10000:1 ratio) | 0.007 mg | Vitamin/Minerals |
| Copper | 50 mg (10:1 ratio) | 5 mg | Vitamin/Minerals |
| Iron | 325 mg | 50 mg | Immune Support - Energy Boost -Vitamin/Minerals |
| Magnesium Oxide | 780 mg | 120 mg | Energy Boost-Blood/Heart Health -Digestive Aid Immune Support |
| Selenium | 40 mg (100:1 ratio) | 0.4 mg | Brain Health - Antioxidant -Fur/Skin/Hair Health |
| Zinc | 65 mg | 10 mg | Fur/Skin/Hair Health - Energy Boost Vitamin/Minerals -Immune Support |
| Potassium Citrate | 3.25 g | 500 mg | Vitamin/Minerals |
| Vitamin K | 50 mg (10:1 ratio) | 5 mg | Blood/Heart Health Vitamin/Minerals |
| Monk Fruit Extract | 3.25 g | 500 mg | Antioxidant - Immune Support |
| L-Leucine | 6.5 g | 1 g | Energy Boost |

The following is an example of a powdered formulation designed as a men’s multivitamin water. The formulation can be diluted with a suitable amount of water and consumed.

TABLE 21

Formulation for Men’s Multivitamin Water

| | | | |
|---|---|---|---|
| Active Ingredient | Weight of nanoparticles (deviation from 6.5:1 Complex:API ratio) | API Dosage | Therapeutic Categories |
| Calcium Lactate | 6.5 g | 1 g | Vitamin/Minerals |
| Chromium | 70 mg (10000:1 ratio) | 0.007 mg | Vitamin/Minerals |
| Iron | 325 mg | 50 mg | Immune Support - Energy Boost -Vitamin/Minerals |
| Magnesium Oxide | 780 mg | 120 mg | Digestive Aid - Energy Boost -Blood/Heart Health - Immune Support |
| Manganese | 65 mg | 10 mg | Vitamin/Minerals |
| Potassium Citrate | 3.25 g | 500 mg | Vitamin/Minerals |
| Vitamin A | 65 mg | 10 mg | Fur/Skin/Hair Health-Vitamin/Minerals - Eye Support |
| Vitamin B1 (Thiamin) | 300 mg (100:1 ratio) | 3 mg | Energy Boost - Vitamin/Minerals |
| Vitamin B2 (Riboflavin) | 100 mg (100:1 ratio) | 1 mg | Energy Boost - Vitamin/Minerals |
| Vitamin B3 (Niacin) | 195 mg | 30 mg | Energy Boost - Vitamin/Minerals -Brain Health |
| Vitamin B6 | 100 mg (100:1 ratio) | 1 mg | Energy Boost - Vitamin/Minerals |
| Vitamin B12 | 50 mg (100:1 ratio) | 0.5 mg | Energy Boost - Vitamin/Minerals -Brain Health |
| Vitamin C | 1.3 g | 200 mg | Antioxidant- Vitamin/Minerals |
| Vitamin D | 50 mg (1000:1 ratio) | 0.05 mg | Antioxidant- Vitamin/Minerals |
| Vitamin K | 50 mg (10:1 ratio) | 5 mg | Blood/Heart Health Vitamin/Minerals |
| Zinc | 65 mg | 10 mg | Fur/Skin/Hair Health - Energy Boost |
| Omega-3 | 3.25 g | 500 mg | Fur/Skin/Hair Health -Immune Support - Brain Health -Antioxidant |
| Selenium | 40 mg (100:1 ratio) | 0.4 mg | Brain Health - Antioxidant -Fur/Skin/Hair Health |
| Methylated Folate | 65 mg | 10 mg | Brain Health |
| Lycopene | 6.5 mg | 1 mg | Antioxidant - Blood/Heart Health |
| Lutein | 65 mg | 10 mg | Antioxidant- Eye Support |
| Coenzyme Q10 | 650 mg | 100 mg | Antioxidant - Brain Health -Blood/Heart Health |
| Astaxanthin | 65 mg | 10 mg | Antioxidant - Immune Support |

The following is an example of a powdered formulation designed as a calming water. The formulation can be diluted with a suitable amount of water and consumed.

TABLE 22

Formulation for Calming

| | | | |
|---|---|---|---|
| Active Ingredient | Weight of nanoparticles (deviation from 6.5:1 Complex:API ratio) | API Dosage | Therapeutic Categories |
| Magnesium Oxide | 780 mg | 120 mg | Digestive Aid - Energy Boost -Blood/Heart Health - Immune Support |
| Vitamin B12 | 50 mg (100:1 ratio) | 0.5 mg | Energy Boost - Vitamin/Minerals - Brain Health |
| Vitamin D | 50 mg (1000:1 ratio) | 0.05 mg | Antioxidant- Vitamin/Minerals |
| Omega-3 | 3.25 g | 500 mg | Fur/Skin/Hair Health -Immune Support - Brain Health -Antioxidant |
| L-Theanine | 1.3 g | 200 mg | Antianxiety |
| Ashwagandha | 3.25 g | 500 mg | Antianxiety |
| Rhodiola Rosea | 650 mg | 100 mg | Antianxiety |
| Gamma-aminobutyric acid (GABA) | 650 mg | 100 mg | Antianxiety |
| Passionflower | 650 mg | 100 mg | Antianxiety |
| Valerian Root | 1.95 g | 300 mg | Antianxiety |
| Licorice Root | 325 mg | 50 mg | Antianxiety |

The following is an example of a powdered formulation designed for seniors (e.g., an anti-aging formula). The formulation can be diluted with a suitable amount of water and consumed.

TABLE 23

Formulation for Seniors/Anti-Aging

| | | | |
|---|---|---|---|
| Active Ingredient | Weight of nanoparticles (deviation from 6.5:1 Complex:API ratio) | API Dosage | Therapeutic Categories |
| L-Taurine | 6.5 g | 1 g | Energy Boost - Brain Health-Blood/Heart Health - Digestive Aid |
| S-adenosyl-L-methionine | 3.25 g | 500 mg | Brain Health - Pain Relief |
| Calcium Lactate | 6.5 g | 1 g | Vitamin/Minerals |
| Acetyl-L-carnitine | 3.25 g | 500 mg | Energy Boost - Brain Health-Blood/Heart Health |
| Vitamin D | 50 mg (1000:1 ratio) | 0.05 mg | Antioxidant- Vitamin/Minerals |
| Vitamin B6 | 100 mg (100:1 ratio) | 1 mg | Energy Boost - Vitamin/Minerals |
| Vitamin B12 | 50 mg (100:1 ratio) | 0.5 mg | Energy Boost - Vitamin/Minerals - Brain Health |
| Beta-Carotene | 65 mg | 10 mg | Antioxidant - Vitamin/Minerals -Eye Support |
| Selenium | 40 mg (100:1 ratio) | 0.4 mg | Brain Health - Antioxidant -Fur/Skin/Hair Health |
| Ginkgo Biloba | 1.3 g | 200 mg | Brain Health |
| Vitamin E | 50 mg | 50 mg | Blood/Heart Health -Immune Support - Antioxidant-Vitamin/Minerals |
| Collagen | 6.75 g (4.5:1 ratio) | 1.5 g | Fur/Skin/Hair Health |
| Omega-3 | 3.25 g | 500 mg | Fur/Skin/Hair Health -Immune Support - Brain Health -Antioxidant |
| Collagen | 6.5 g | 1 g | Fur/Skin/Hair Health |
| Peppermint Oil | 1.625 g | 250 mg | Antioxidant- Fur/Skin/Hair Health |
| Citicoline | 3.25 g | 500 mg | Brain Health |
| Coenzyme Q10 | 650 mg | 100 mg | Antioxidant - Brain Health -Blood/Heart Health |
| Phosphatidylserine | 1.3 g | 200 mg | Brain Health |
| Saw Palmetto | 1.3 g | 200 mg | Fur/Skin/Hair Health |
| Curcumin | 650 mg | 100 mg | Antioxidant |

The following is an example of a powdered formulation designed for brain health and/or mental focus. The formulation can be diluted with a suitable amount of water and consumed.

TABLE 24

Formulation for Brain Health/Mental Focus

| | | | |
|---|---|---|---|
| Active Ingredient | Weight of nanoparticles (deviation from standard 6.5:1 Complex:API ratio) | API Dosage | Therapeutic Categories |
| S-adenosyl-L-methionine | 3.25 g | 500 mg | Brain Health - Pain Relief |
| Pomegranate Extract | 6.5 g | 1 g | Antioxidant - Brain Health -Blood/Heart Health |
| Rhodiola Rosea | 650 mg | 100 mg | Brain Health - Antianxiety |
| L-Tyrosine | 6.5 g | 1 g | Brain Health |
| Vitamin B1 (Thiamin) | 300 mg (100:1 ratio) | 3 mg | Energy Boost -Vitamin/Minerals |
| Vitamin B6 | 100 mg (100:1 ratio) | 1 mg | Energy Boost -Vitamin/Minerals |
| Vitamin B9 (Folic Acid) | 50 mg (100:1 ratio) | 0.5 mg | Energy Boost -Vitamin/Minerals - Brain Health - Kidney/Urinary Tract Health |
| Vitamin B12 | 50 mg (100:1 ratio) | 0.5 mg | Energy Boost -Vitamin/Minerals - Brain Health |
| Gingko Biloba | 1.3 g | 200 mg | Brain Health |
| Omega-3 | 3.25 g | 500 mg | Fur/Skin/Hair Health -Immune Support - Brain Health -Antioxidant |
| Creatine | 6.5 g | 1 g | Energy Boost - Brain Health-Blood/Heart Health |
| Citocoline | 3.25 g | 500 mg | Brain Health |
| Phosphatidylserine | 1.3 g | 200 mg | Brain Health |
| Acetyl-L-carnitine | 3.25 g | 500 mg | Energy Boost - Brain Health-Blood/Heart Health |
| Green Tea Extract (Source of Caffeine) | 1.625 g | 250 mg | Brain Health |
| Bacopa Monnieri | 650 mg | 100 mg | Brain Health |

The following is an example of a powdered formulation designed for joint health. The formulation can be diluted with a suitable amount of water and consumed.

TABLE 25

Formulation for Joint Health

| | | | |
|---|---|---|---|
| Active Ingredient | Weight of nanoparticles (deviation from 6.5:1 Complex:API ratio) | API Dosage | Therapeutic Categories |
| S-adenosyl-L-methionine | 3.25 g | 500 mg | Brain Health - Pain Relief |
| Glucosamine | 3.25 g | 500 mg | Antioxidant - Pain Relief |
| Chondroitin Sulfate | 3.9 g | 600 mg | Antioxidant - Pain Relief |
| Hyaluronic Acid | 650 mg | 100 mg | Pain Relief - Fur/Skin/Hair Health |
| Collagen | 6.5 g | 1 g | Fur/Skin/Hair Health |
| MSM | 1.3 g | 200 mg | Antioxidant - Pain Relief |
| Curcumin | 650 mg | 100 mg | Antioxidant |
| Devil’s Claw | 3.25 g | 500 mg | Antioxidant |
| Turmeric | 650 mg | 100 mg | Antioxidant |
| Keratin | 1.625 g | 250 mg | Pain Relief - Fur/Skin/Hair Health |
| Peppermint Oil | 1.625 g | 250 mg | Antioxidant- Fur/Skin/Hair Health |
| Hops Flowers (Humulus Lupulus) | 650 mg | 100 mg | Antioxidant |
| Boswellia | 1.3 g | 200 mg | Antioxidant |
| Omega-3 | 3.25 g | 500 mg | Fur/Skin/Hair Health -Immune Support - Brain Health -Antioxidant |
| Krill Oil | 3.25 g | 500 mg | Antioxidant - Pain Relief -Blood/Heart Health |

The following is an example of a powdered formulation designed for hair loss. The formulation can be diluted with a suitable amount of water and consumed.

TABLE 26

Formulation for Hair Loss

| | | | |
|---|---|---|---|
| Active Ingredient | Weight of nanoparticles (deviation from 6.5:1 Complex:API ratio) | API Dosage | Therapeutic Categories |
| Vitamin B6 | 100 mg (100:1 ratio) | 1 mg | Energy Boost - Vitamin/Minerals |
| Vitamin C | 1.3 g | 200 mg | Antioxidant- Vitamin/Minerals |
| Vitamin E | 50 mg | 50 mg | Fur/Skin/Hair Health -Vitamin/Minerals - Antioxidant |
| Vitamin B7 (Biotin) | 100 mg (100:1 ratio) | 1 mg | Fur/Skin/Hair Health - Energy Boost - Vitamin/Minerals |
| Gotu Kola | 3.25 g | 500 mg | Brain Health |
| Goji Berry | 975 mg | 150 mg | Antioxidant - Immune Support - Eye Support |
| Fo-Ti Root | 6.5 g | 1 g | Fur/Skin/Hair Health |
| Bladderwrack | 1.625 g | 250 mg | Antioxidant |
| Nori Yaki | 6.5 g | 1 g | Antioxidant |
| MSM | 1.3 g | 200 mg | Antioxidant - Pain Relief |
| Collagen | 6.5 g | 1 g | Fur/Skin/Hair Health |
| Keratin | 1.625 g | 250 mg | Fur/Skin/Hair Health |
| Hyaluronic Acid | 650 mg | 100 mg | Pain Relief - Fur/Skin/Hair Health |
| Astaxanthin | 65 mg | 10 mg | Antioxidant - Immune Support |
| Calcium | 6.5 g | 1 g | Vitamin/Minerals |
| Peppermint Oil | 1.625 g | 250 mg | Antioxidant- Fur/Skin/Hair Health |
| Zinc | 65 mg | 10 mg | Fur/Skin/Hair Health - Energy Boost - Vitamin/Minerals -Immune Support |
| Selenium | 40 mg (100:1 ratio) | 0.4 mg | Brain Health - Antioxidant -Fur/Skin/Hair Health |
| Reishi Mushroom | 6.5 g | 1 g | Fur/Skin/Hair Health - Immune Support |
| Copper | 50 mg (10:1 ratio) | 5 mg | Vitamin/Minerals |
| Manganese | 65 mg | 10 mg | Vitamin/Minerals |

The following is an example of a powdered formulation designed to help digestion. The formulation can be diluted with a suitable amount of water and consumed.

TABLE 27

Formulation for Digestive Aid

| | | | |
|---|---|---|---|
| Active Ingredient | Weight of nanoparticles (deviation from 6.5:1 Complex:API ratio) | API Dosage | Therapeutic Categories |
| Aluminum hydroxide | 2.6 g | 400 mg | Digestive Aid -Kidney/Urinary Tract Health |
| Magnesium | 780 mg | 120 mg | Digestive Aid - Energy Boost -Blood/Heart Health - Immune Support |
| Licorice Root | 325 mg | 50 mg | Digestive Aid |
| Peppermint Oil | 1.625 g | 250 mg | Antioxidant- Fur/Skin/Hair Health -Digestive Aid |
| Chamomile | 13 g | 2 g | Antianxiety |
| Ginger root extract | 6.5 g | 1 g | Antioxidant - Digestive Aid |
| L-Taurine | 6.5 g | 1 g | Energy Boost - Brain Health-Blood/Heart Health - Digestive Aid |
| Psyllium | 6.5 g | 1 g | Digestive Aid |
| Yucca root | 6.5 g | 1 g | Digestive Aid |
| S-adenosyl-L-methionine | 3.5 g | 500 mg | Brain Health - Pain Relief |

The following is an example of a powdered formulation designed to support the growth of children. The formulation can be diluted with a suitable amount of water and consumed.

TABLE 28

Formulation for Kids Growth Support

| | | | |
|---|---|---|---|
| Active Ingredient | Weight of nanoparticles (deviation from 6.5:1 Complex:API ratio) | API Dosage | Therapeutic Categories |
| Colostrum | 1.3 g | 200 mg | Immune Support - Blood/Heart Health - Energy Boost |
| Calcium | 1.3 g | 200 mg | Vitamin/Minerals |
| Vitamin D | 30 mg (1000:1 ratio) | 0.03 mg | Fur/Skin/Hair Health -Vitamin/Minerals |
| Vitamin K | 100 mg (100:1 ratio) | 1 mg | Blood/Heart Health |
| L-Arginine | 325 mg | 50 mg | Brain Health - Blood/Heart Health |
| Zinc | 50 mg (10:1 ratio) | 5 mg | Fur/Skin/Hair Health - Energy Boost - Vitamin/Minerals -Immune Support |
| Omega-3/Fish Oils | 650 mg | 100 mg | Fur/Skin/Hair Health - Immune Support - Blood/Heart Health -Antioxidant |
| Beetroot Extract | 455 mg | 70 mg | Immune Support - Antioxidant -Energy Boost |
| Phosphatidylserine | 195 mg | 30 mg | Brain Health |
| Iron | 30 mg (10:1 ratio) | 3 mg | Immune Support - Energy Boost -Vitamin/Minerals |
| Vitamin A | 200 mg (1000:1 ratio) | 0.2 mg | Fur/Skin/Hair Health-Vitamin/Minerals - Eye Support |
| Ginseng | 455 mg | 70 mg | Antioxidant - Energy Boost |
| Collagen Hydrolysate | 80 mg (100:1 ratio) | 0.8 mg | Fur/Skin/Hair Health |
| Iodine | 25 mg (1000:1 ratio) | 0.025 mg | Immune Support |
| Gingko Biloba | 150 mg (10:1 ratio) | 15 mg | immune Support - Brain Health |

The following is an example of a powdered formulation designed to increase energy. The formulation can be diluted with a suitable amount of water and consumed.

TABLE 29

Formulation for Energy Increase

| | | | |
|---|---|---|---|
| Active Ingredient | Weight of nanoparticles (deviation from 6.5:1 Complex:API ratio) | API Dosage | Therapeutic Categories |
| Peppermint Oil | 1.625 g | 250 mg | Antioxidant- Fur/Skin/Hair Health - Digestive Aid |
| L-Carnitine | 6.5 g | 1 g | Energy Boost - Brain Health-Blood/Heart Health |
| Ashwagandha | 0.975 g | 150 mg | Antioxidant - Energy Boost |
| Rhodiola Rosea | 650 mg | 100 mg | Brain Health - Energy Boost |
| Vitamin B12 | 0.05 g (100:1 ratio) | 0.5 mg | Energy Boost -Vitamin/Minerals - Brain Health |
| Iron | 0.325 g | 50 mg | Immune Support - Energy Boost -Vitamin/Minerals |
| Coenzyme Q10 (CoQ10) | 0.65 g | 100 mg | Antioxidant - Brain Health -Blood/Heart Health |
| Creatine | 0.65 g | 100 mg | Energy Boost - Brain Health-Blood/Heart Health |
| Beta Alanine | 1.3 g | 200 mg | Antioxidant - Energy Boost |
| L-Citrulline | 0.65 g | 100 mg | Energy Boost - Brain Health-Blood/Heart Health |
| Beetroot Extract | 1.95 g | 300 mg | Immune Support - Antioxidant -Energy Boost |
| L-Theanine | 1.3 g | 200 mg | Antianxiety |
| Tyrosine | 3.25 g | 500 mg | Energy Boost |
| Green Tea Extract | 1.3 g | 200 mg | Antioxidant - Energy Boost |

The following is an example of a powdered formulation designed to support immune function. The formulation can be diluted with a suitable amount of water and consumed.

TABLE 30

Formulation for Immune Support

| | | | |
|---|---|---|---|
| Active Ingredient | Weight of nanoparticles (deviation from 6.5:1 Complex:API ratio) | API Dosage | Therapeutic Categories |
| Pomegranate Extract | 6.5 g | 1 g | Antioxidant - Brain Health -Blood/Heart Health |
| Goji Berry | 975 mg | 150 mg | Immune Support - Antioxidant |
| Peppermint Oil | 1.625 g | 250 mg | Antioxidant- Fur/Skin/Hair Health -Digestive Aid |
| Echinacea | 6.5 g | 1 g | Immune Support - Energy Boost |
| Beetroot Extract | 1.95 g | 300 mg | Immune Support - Antioxidant -Energy Boost |
| Astaxanthin | 65 mg | 10 mg | Immune Support |
| Zinc | 65 mg | 10 mg | Fur/Skin/Hair Health - Energy Boost - Vitamin/Minerals -Immune Support |
| Iron | 325 mg | 50 mg | Immune Support - Energy Boost -Vitamin/Minerals |
| Magnesium | 780 mg | 120 mg | Digestive Aid - Energy Boost -Blood/Heart Health - Immune Support |
| Quercetin | 1.3 g | 200 mg | Antioxidant |
| Vitamin A | 65 mg | 10 mg | Fur/Skin/Hair Health-Vitamin/Minerals - Eye Support |
| Vitamin C | 1.3 g | 200 mg | Antioxidant- Vitamin/Minerals |
| Vitamin D | 50 mg (1000:1 ratio) | 0.05 mg | Fur/Skin/Hair Health -Vitamin/Minerals |
| Vitamin E | 50 mg | 50 mg | Fur/Skin/Hair Health -Vitamin/Minerals - Antioxidant |

The following is an example of a powdered formulation designed to help with allergies. The formulation can be diluted with a suitable amount of water and consumed.

TABLE 31

Formulation for Allergies Support

| | | | |
|---|---|---|---|
| Active Ingredient | Weight of nanoparticles (deviation from 6.5:1 Complex:API ratio) | API Dosage | Therapeutic Categories |
| Goji Berry | 975 mg | 150 mg | Immune Support - Antioxidant |
| | | | Antioxidant- Fur/Skin/Hair Health - |
| Peppermint Oil | 1.625 g | 250 mg | Digestive Aid |
| Echinacea | 3.25 g | 500 mg | Immune Support - Energy Boost |
| Grape Seed Extract | 650 mg | 100 mg | Immune Support - Antioxidant |
| Pycnogenol | 325 mg | 50 mg | Antioxidant |
| Goldenseal | 1.3 g | 200 mg | Antioxidant |
| Spirulina | 3.25 g | 500 mg | Immune Support - Antioxidant-Blood/Heart Health |
| Quercetin | 1.3 g | 200 mg | Antioxidant |
| Vitamin C | 1.3 g | 200 mg | Antioxidant- Vitamin/Minerals |
| Omega3 | 3.25 g | 500 mg | Fur/Skin/Hair Health Immune Support - Blood/Heart Health - Antioxidant |

The following is an example of a powdered formulation designed to support eye function. The formulation can be diluted with a suitable amount of water and consumed.

TABLE 32

Formulation for Eye Support

| | | | |
|---|---|---|---|
| Active Ingredient | Weight of nanoparticles (deviation from 6.5:1 Complex:API ratio) | API Dosage | Therapeutic Categories |
| Goji Berry | 975 mg | 150 mg | Immune Support -Antioxidant |
| Bilberry Extract | 1.3 g | 200 mg | Eye Support -Blood/Heart Health |
| Beta-Carotene | 65 mg | 10 mg | Vitamin/Minerals - Eye Support - Antioxidant |
| Vitamin A | 65 mg | 10 mg | Fur/Skin/Hair Health-Vitamin/Minerals - Eye Support |
| Vitamin C | 1.3 g | 200 mg | Antioxidant-Vitamin/Minerals |
| Vitamin D | 50 mg (1000:1 ratio) | 0.05 mg | Fur/Skin/Hair Health -Vitamin/Minerals |
| Vitamin E | 50 mg | 50 mg | Fur/Skin/Hair Health -Vitamin/Minerals -Antioxidant |
| Lutein | 65 mg | 10 mg | Eye Support-Antioxidant |
| Zeaxanthin | 200 mg (100:1 ratio) | 2 mg | Eye Support |

The following is an example of a powdered formulation designed to support weight loss. The formulation can be diluted with a suitable amount of water and consumed.

TABLE 33

Formulation for Weight Loss

| | | | |
|---|---|---|---|
| Active Ingredient | Weight of nanoparticles (deviation from 6.5:1 Complex:API ratio) | API Dosage | Therapeutic Categories |
| Choline | 1.3 g | 200 mg | Weight Loss- Digestive Aid |
| CLA | 6.5 g | 1 g | Weight Loss |
| Guarana | 1.3 g | 200 mg | Antioxidant - Energy Boost -Weight Loss |
| Garcinia Cambodia | 3.25 g | 500 mg | Weight Loss |
| Psyllium | 6.5 g | 1 g | Digestive Aid |
| Collagen | 6.75 g (4.5:1 ratio) | 1.5 g | Fur/Skin/Hair Health |
| Omega 3 | 1.625 g | 250 mg | Fur/Skin/Hair Health Immune Support - Blood/Heart Health -Antioxidant |
| L-Carnitine | 6.5 g | 1 g | Energy Boost -Weight Loss - Brain Health |

The following is an example of a powdered formulation designed to support weight gain. The formulation can be diluted with a suitable amount of water and consumed.

TABLE 34

Formulation for Weight Gain

| | | | |
|---|---|---|---|
| Active Ingredient | Weight of nanoparticles (deviation from 6.5:1 Complex:API ratio) | API Dosage | Therapeutic Categories |
| Whey Protein | 6.5 g | 1 g | |
| Creatine | 6.5 g | 1 g | Energy Boost -Weight Gain - Brain Health |
| Beta Alanine | 1.3 g | 200 mg | Antioxidant - Energy Boost |
| Sunflower Oil | 3.25 g | 500 mg | Fur/Skin/Hair Health -Weight Gain |
| Monk Fruit | 3.25 g | 500 mg | Antioxidant |
| Beta Hydroxy Beta-Methylbutyrate | 1.3 g | 200 mg | Energy Boost |
| Ginger Root | 0.975 g | 150 mg | Antioxidant - Digestive Aid |

The following is an example of a powdered formulation designed to support blood and heart function. The formulation can be diluted with a suitable amount of water and consumed.

TABLE 35

Formulation for Blood and Heart Support.

| | | | |
|---|---|---|---|
| Active Ingredient | Weight of nanoparticles (deviation from 6.5:1 Complex:API ratio) | API Dosage | Therapeutic Categories |
| L-Taurine | 6.5 g | 1 g | Energy Boost - Blood/Heart Health -Brain Health |
| Pomegranate Extract | 6.5 g | 1 g | Antioxidant - Brain Health - Blood/Heart Health |
| Omega-3 | 3.25 g | 500 mg | Fur/Skin/Hair Health Immune Support -Blood/Heart Health - Antioxidant |
| Bilberry Extract | 1.3 g | 200 mg | Eye Support - Blood/Heart Health |
| Magnesium | 780 mg | 120 mg | Digestive Aid - Energy Boost -Blood/Heart Health - Immune Support |
| Vitamin B3 (Niacin) | 195 mg | 30 mg | Energy Boost - Vitamin/Minerals -Blood/Heart Health |
| Vitamin B9 (Folic Acid) | 50 mg (100:1 ratio) | 0.5 mg | Vitamin/Minerals - Blood/Heart Health -Kidney/Urinary Tract HealthVitamin/Minerals - Blood/Heart Health |
| Vitamin B12 | 50 mg (100:1 ratio) | 0.5 mg | Energy Boost - Vitamin/Minerals - Brain Health |
| Vitamin E | 50 mg | 50 mg | Fur/Skin/Hair Health - Vitamin/Minerals -Antioxidant |
| Coenzyme Q10 | 650 mg | 100 mg | Antioxidant - Brain Health - Blood/Heart Health |

The following is an example of a powdered formulation designed to support kidney and urinary tract function. The formulation can be diluted with a suitable amount of water and consumed.

TABLE 36

Formulation for Kidney and Urinary Tract Support.

| | | | |
|---|---|---|---|
| Active Ingredient | Weight of nanoparticles (deviation from 6.5:1 Complex:API ratio) | API Dosage | Therapeutic Categories |
| Aluminum hydroxide | 2.6 g | 400 mg | Digestive Aid -Kidney/Urinary Tract Health |
| Astragalus root powder | 6.5 g | 1 g | Kidney/Urinary Tract Health |
| Choline | 1.3 g | 200 mg | Weight Loss- Kidney/Urinary Tract Health |
| Dong Quai Root | 6.5 g | 1 g | Kidney/Urinary Tract Health |
| Nettle root extract | 1.625 g | 250 mg | Kidney/Urinary Tract Health |
| Vitamin B9 (Folic Acid) | 50 mg (100:1 ratio) | 0.5 mg | Vitamin/Minerals - Blood/Heart Health - Kidney/Urinary Tract Health |
| Iron | 325 mg | 50 mg | Immune Support - Energy Boost -Vitamin/Minerals |

The following is an example of a powdered formulation designed to support women during pregnancy. The formulation can be diluted with a suitable amount of water and consumed.

TABLE 37

Formulation for Pregnancy Support

| | | | |
|---|---|---|---|
| Active Ingredient | Weight of nanoparticles (deviation from 6.5:1 Complex:API ratio) | API Dosage | Therapeutic Categories |
| Iodine | 50 mg (1000:1 ratio) | 0.05 mg | Immune Support |
| Iron | 0.325 g | 50 mg | Immune Support - Energy Boost -Vitamin/Minerals |
| Zinc | 65 mg | 10 mg | Fur/Skin/Hair Health - Energy Boost -Vitamin/Minerals -Immune Support |
| Vitamin B1 (Thiamin) | 300 mg (100:1 ratio) | 3 mg | Energy Boost - Vitamin/Minerals |
| Vitamin B3 (Niacin) | 195 mg | 30 mg | Energy Boost - Vitamin/Minerals -Blood/Heart Health |
| Vitamin B6 | 100 mg (100:1 ratio) | 1 mg | Energy Boost - Vitamin/Minerals -Brain Health |
| Vitamin B9 (Folic Acid) | 50 mg (100:1 ratio) | 0.5 mg | Vitamin/Minerals - Blood/Heart Health - Kidney/Urinary Tract Health |
| Vitamin B12 | 50 mg (100:1 ratio) | 0.5 mg | Energy Boost - Vitamin/Minerals -Brain Health |
| Vitamin D | 50 mg (1000:1 ratio) | 0.05 mg | Fur/Skin/Hair Health -Vitamin/Minerals |
| .Vitamin A | 65 mg | 10 mg | Fur/Skin/Hair Health-Vitamin/Minerals - Eye Support |
| Vitamin C | 1.3 g | 200 mg | Antioxidant- Vitamin/Minerals |
| Vitamin E | 50 mg | 50 mg | Fur/Skin/Hair Health -Vitamin/Minerals - Antioxidant |
| DHA | 0.65 g | 100 mg | Fur/Skin/Hair Health Immune Support - Blood/Heart Health - Antioxidant |
| Choline | 1.3 g | 200 mg | Weight Loss- Kidney/Urinary Tract Health |
| Calcium | 3.25 g | 500 mg | Vitamin/Minerals |
| Magnesium | 780 mg | 120 mg | Digestive Aid - Energy Boost -Blood/Heart Health - Immune Support |

The following is an example of a powdered formulation designed for smoking abstinence. The formulation can be diluted with a suitable amount of water and consumed.

TABLE 38

Formulation for Smoking Abstinence.

| | | | |
|---|---|---|---|
| Active Ingredient | Weight of nanoparticles (deviation from 6.5:1 Complex:API ratio) | API Dosage | Therapeutic Categories |
| S-adenosyl-L-methionine | 3.25 g | 500 mg | Brain Health - Pain Relief |
| Vitamin B1 (Thiamin) | 300 mg (100:1 ratio) | 3 mg | Energy Boost - Vitamin/Minerals |
| Vitamin B2 (Riboflavin) | 100 mg (100:1 ratio) | 1 mg | Energy Boost - Vitamin/Minerals |
| Vitamin B3 (Niacin) | 195 mg | 30 mg | Energy Boost - Vitamin/Minerals -Brain Health |
| Vitamin B5 (Pantothenic acid) | 650 mg | 100 mg | Energy Boost - Vitamin/Minerals |
| Vitamin B6 | 100 mg (100:1 ratio) | 1 mg | Energy Boost - Vitamin/Minerals |
| Vitamin B9 (Folic Acid) | 50 mg (100:1 ratio) | 0.5 mg | Vitamin/Minerals - Blood/Heart Health - Kidney/Urinary Tract Health |
| Vitamin B12 | 50 mg (100:1 ratio) | 0.5 mg | Energy Boost - Vitamin/Minerals -Brain Health |
| Vitamin C | 1.3 g | 200 mg | Antioxidant- Vitamin/Minerals |
| Black Pepper Extract | 1.3 g | 200 mg | Antioxidant - Brain Health |
| Asian Ginseng | 0.975 g | 150 mg | Energy Boost |
| Calcium | 6.5 g | 1 g | Vitamin/Minerals |
| Magnesium | 780 mg | 120 mg | Digestive Aid - Energy Boost -Blood/Heart Health - Immune Support |
| Beta-Carotene | 65 mg | 10 mg | Vitamin/Minerals - Eye Support -Antioxidant |

The following is an example of a powdered formulation designed to support sexual function. The formulation can be diluted with a suitable amount of water and consumed.

TABLE 39A

Formulation for Sexual Function Support.

| | | | |
|---|---|---|---|
| Active Ingredient | Weight of nanoparticles (deviation from 6.5:1 Complex:API ratio) | API Dosage | Therapeutic Categories |
| Vitamin B3 (Niacin) | 195 mg | 30 mg | Energy Boost -Vitamin/Minerals - Brain Health |
| Hops Flowers (Humulus Lupulus) | 650 mg | 100 mg | Sexual Function Support |
| Ginko Biloba | 1.3 g | 200 mg | Brain Health |
| Mucuna Pruruiens | 3.25 g | 500 mg | Sexual Function Support |
| Epimedium Sagittat (Horny Goat Weed) | 3.25 g | 500 mg | Sexual Function Support |
| Cayenne Extract | 1.3 g | 200 mg | Sexual Function Support |
| Black Pepper Extract | 1.3 g | 200 mg | Antioxidant - Brain Health |
| Ashwagandha | 0.975 g | 150 mg | Energy Boost |
| Damiana Extract | 3.25 g | 500 mg | Sexual Function Support |
| Maca | 3.25 g | 500 mg | Antioxidant -Energy Boost |
| L-Arginine | 0.65 g | 100 mg | Brain Health - Blood/Heart Health |

A similar powder formulation is listed in Table 39B. In this case some ingredients are removed due to pharmaceutical incompatibility with Yohimbe.

TABLE 39B

Formulation for Sexual Function Support.

| | | | |
|---|---|---|---|
| Active Ingredient | Weight of nanoparticles (deviation from 6.5:1 Complex:API ratio) | API Dosage | Therapeutic Categories |
| Yohimbe | 65 mg | 10 mg | Sexual Function Support |
| Vitamin B3 (Niacin) | 195 mg | 30 mg | Energy Boost -Vitamin/Minerals - Brain Health |
| L-Arginine | 0.65 g | 100 mg | Brain Health - Blood/Heart Health |
| Ginko Biloba | 1.3 g | 200 mg | Brain Health |
| Epimedium Sagittat (Horny Goat Weed) | 3.25 g | 500 mg | Sexual Function Support |

The following is an example of a powdered formulation designed to support e-gaming athletes. The formulation can be diluted with a suitable amount of water and consumed.

TABLE 40

Formulation for e-Gaming Athletes

| | | | |
|---|---|---|---|
| Active Ingredient | Weight of nanoparticles (deviation from 6.5:1 Complex:API ratio) | API Dosage | Therapeutic Categories |
| L-Theanine | 0.65 g | 100 mg | Antianxiety |
| Tyrosine | 3.25 g | 500 mg | Energy Boost - Brain Health |
| Omega 3-Fatty Acids | 3.25 g | 500 mg | Fur/Skin/Hair Health Immune Support - Blood/Heart Health -Antioxidant |
| Tryptophan | 1.3 g | 200 mg | Energy Boost - Brain Health |
| Vitamin B1 (Thiamin) | 300 mg (100:1 ratio) | 3 mg | Energy Boost - Vitamin/Minerals |
| Vitamin B2 (Riboflavin) | 100 mg (100:1 ratio) | 1 mg | Energy Boost - Vitamin/Minerals |
| Vitamin B3 (Niacin) | 195 mg | 30 mg | Energy Boost - Vitamin/Minerals -Brain Health |
| Vitamin B5 (Pantothenic acid) | 650 mg | 100 mg | Energy Boost - Vitamin/Minerals |
| Vitamin B6 | 100 mg (100:1 ratio) | 1 mg | Energy Boost - Vitamin/Minerals |
| Vitamin B9 (Folic Acid) | 50 mg (100:1 ratio) | 0.5 mg | Energy Boost - Vitamin/Minerals -Kidney/Urinary Tract Health |
| Vitamin B12 | 50 mg (100:1 ratio) | 0.5 mg | Energy Boost - Vitamin/Minerals -Brain Health |
| Vitamin C | 1.3 g | 200 mg | Antioxidant- Vitamin/Minerals |
| Vitamin D | 50 mg (1000:1 ratio) | 0.05 mg | Antioxidant- Vitamin/Minerals |
| Vitamin E | 50 mg | 50 mg | Blood/Heart Health -Immune Support - Antioxidant-Vitamin/Minerals |
| Ginko Biloba | 1.3 g | 200 mg | Energy Boost - Brain Health |
| Bacopa Monnieri | 650 mg | 100 mg | Brain Health |
| Resveratrol | 1.625 g | 250 mg | Antioxidant |
| Green Tea Extract | 1.3 g | 200 mg | Antioxidant -Energy Boost |
| Ashwagandha | 0.975 g | 150 mg | Antioxidant -Energy Boost |
| Choline | 1.3 g | 200 mg | Weight Loss- Kidney/Urinary Tract Health |
| Creatine | 1.625 g | 250 mg | Energy Boost - Brain Health |
| Theacrine | 0.975 g | 150 mg | Energy Boost - Brain Health |
| Zinc | 65 mg | 10 mg | Fur/Skin/Hair Health - Energy Boost - Vitamin/Minerals -Immune Support |
| Asian Ginseng | 0.975 g | 150 mg | Antioxidant -Energy Boost |
| Curcumin | 650 mg | 100 mg | Antioxidant |
| L-Leucine | 1.3 g | 200 mg | Energy Boost - Brain Health |
| Rhodiola Rosea | 650 mg | 100 mg | Brain Health -Antianxiety |

The following is an example of a powdered formulation designed for a water for the improvement of a pet’s (e.g., a dog) skin and hair health. The formulation can be diluted with a suitable amount of water and consumed.

TABLE 41

Formulation for Skin and Hair Pet Water

| Active Ingredient | Weight of nanoparticles (deviation from 6.5:1 Complex:API ratio) | API Dosage | Therapeutic Categories |
|---|---|---|---|
| Fish Oil | 650 mg | 100 mg | Fur/Skin/Hair Health Immune Support - Brain Health - Antioxidant - Pain Relief |
| Vitamin A | 65 mg | 10 mg | Fur/Skin/Hair Health-Vitamin/Minerals - Eye Support |
| Vitamin B5 (Pantothenic acid) | 65 mg | 10 mg | Fur/Skin/Hair Health -Vitamin/Minerals |
| Vitamin D | 200 mg (1000:1 ratio) | 0.02 mg | Fur/Skin/Hair Health -Vitamin/Minerals |
| Vitamin E | 1.95 g | 300 mg | Fur/Skin/Hair Health -Vitamin/Minerals - Antioxidant |
| Chamomile | 19.5 g | 3 g | Antianxiety |
| Primrose oil | 3.25 g | 500 mg | Fur/Skin/Hair Health |
| Sunflower oil | 3.25 g | 500 mg | Fur/Skin/Hair Health |
| Zinc | 32.5 mg | 5 mg | Fur/Skin/Hair Health - Energy Boost - Vitamin/Minerals -Immune Support |
| Turmeric | 487.5 mg | 75 mg | Antioxidant - Immune Support |
| Hyaluronic Acid | 130 mg | 20 mg | Pain Relief - Fur/Skin/Hair Health |
| Vitamin B7 (Biotin) | 100 mg (100:1 ratio) | 1 mg | Fur/Skin/Hair Health - Energy Boost - Vitamin/Minerals |

The following is an example of a powdered formulation designed for a multivitamin pet water. The formulation can be diluted with a suitable amount of water and consumed.

TABLE 42

Formulation for Multivitamin Pet Water

| | | | |
|---|---|---|---|
| Active Ingredient | Weight of nanoparticles (deviation from 6.5:1 Complex:API ratio) | API Dosage | Therapeutic Categories |
| Glucosamine | 650 mg | 100 mg | Antioxidant - Pain Relief |
| Coenzyme Q10 | 500 mg (100:1 ratio) | 5 mg | Antioxidant - Brain Health -Blood/Heart Health |
| Vitamin B1 (Thiamin) | 300 mg (100:1 ratio) | 3 mg | Energy Boost - Vitamin/Minerals |
| Vitamin B2 (Riboflavin) | 65 mg | 10 mg | Energy Boost - Vitamin/Minerals |
| Vitamin B3 (Niacin) | 325 mg | 50 mg | Energy Boost - Vitamin/Minerals |
| Vitamin B6 | 325 mg | 50 mg | Energy Boost - Vitamin/Minerals |
| Vitamin B12 | 200 mg (100:1 ratio) | 0.2 mg | Energy Boost - Vitamin/Minerals |
| Vitamin C | 65 mg | 10 mg | Antioxidant- Vitamin/Minerals |
| Vitamin E | 1.95 g | 300 mg | Blood/Heart Health -Immune Support - Antioxidant-Vitamin/Minerals |
| B-Carotene | 162 mg | 25 mg | Antioxidant- Vitamin/Minerals |
| Milk Thistle | 130 mg | 20 mg | Antioxidant - Immune Support |
| Ginger Root Extract | 325 mg | 50 mg | Antioxidant - Digestive Aid |
| Vitamin D | 200 mg (1000:1 ratio) | 0.02 mg | Antioxidant- Vitamin/Minerals |
| Fish Oil | 650 mg | 100 mg | Immune Support - Brain Health -Antioxidant - Pain Relief -Fur/Skin/Hair Health |
| CBD | 500 mg (100:1 ratio) | 5 mg | Antioxidant - Antianxiety- Pain Relief |

The following is an example of a powdered formulation designed for a calming pet water. The formulation can be diluted with a suitable amount of water and consumed.

TABLE 43

Formulation for Calming Pet Water

| | | | |
|---|---|---|---|
| Active Ingredient | Weight of nanoparticles (deviation from 6.5:1 Complex:API ratio) | API Dosage | Therapeutic Categories |
| L-Trytophan | 65 mg | 10 mg | Antianxiety |
| Alpha-Casozephine (Zylkene) | 500 mg (100:1 ratio) | 5 mg | Antianxiety |
| Chamomile | 19.5 g | 3 g | Antianxiety |
| CBD | 500 mg (100:1 ratio) | 5 mg | Antioxidant - Antianxiety-Pain Relief |
| Melatonin | 100 mg (100:1 ratio) | 1 mg | Antianxiety |
| L-Theanine | 65 mg | 10 mg | Antianxiety |
| Valerian Root | 130 mg | 20 mg | Antianxiety |

The following is an example of a powdered formulation designed for a senior/anti-aging pet water. The formulation can be diluted with a suitable amount of water and consumed.

TABLE 44

Formulation for Senior/Anti-Aging Pet Water

| | | | |
|---|---|---|---|
| Active Ingredient | Weight of nanoparticles (deviation from 6.5:1 Complex:API ratio) | API Dosage | Therapeutic Categories |
| Vitamin A | 65 mg | 10 mg | Antioxidant- Vitamin/Minerals -Eye Support |
| Vitamin B1 (Thiamin) | 300 mg (100:1 ratio) | 3 mg | Energy Boost - Vitamin/Minerals -Brain Health |
| Vitamin B2 (Riboflavin) | 65 mg | 10 mg | Energy Boost - Vitamin/Minerals |
| Vitamin B6 | 325 mg | 50 mg | Energy Boost - Vitamin/Minerals |
| Vitamin B12 | 200 mg (100:1 ratio) | 0.2 mg | Energy Boost - Vitamin/Minerals -Brain Health |
| Vitamin C | 65 mg | 10 mg | Antioxidant- Vitamin/Minerals |
| Vitamin E | 1.95 g | 300 mg | Blood/Heart Health -Immune Support - Antioxidant-Vitamin/Minerals |
| B-Carotene | 162 mg | 25 mg | Antioxidant- Vitamin/Minerals- Eye Support |
| alpha-lipoic acid | 65 mg | 10 mg | Antioxidant -Brain Health |
| L-Taurine | 3.25 g | 500 mg | Energy Boost - Brain Health-Blood/Heart Health - Digestive Aid |
| L-Carnitine | 1.3 g | 200 mg | Energy Boost - Brain Health-Blood/Heart Health |
| S-adenosyl-L-methionine | 325 mg | 50 mg | Brain Health - Pain Relief |
| Turmeric | 325 mg | 50 mg | Antioxidant - Immune Support |
| CBD | 500 mg (100:1 ratio) | 5 mg | Antioxidant - Antianxiety- Pain Relief |
| Coenzyme Q10 | 500 mg (100:1 ratio) | 5 mg | Antioxidant - Brain Health -Blood/Heart Health |
| Pomegranate Extract | 1.3 g | 200 mg | Antioxidant - Brain Health -Blood/Heart Health |
| Quercetin | 325 mg | 50 mg | Antioxidant - Brain Health |

The following is an example of a powdered formulation designed for brain health/mental focus. The formulation can be diluted with a suitable amount of water and consumed.

TABLE 45

Formulation for Brain Health/Mental Focus

| | | | |
|---|---|---|---|
| Active Ingredient | Weight of nanoparticles (deviation from 6.5:1 Complex:API ratio) | API Dosage | Therapeutic Categories |
| Vitamin B1 (Thiamin) | 300 mg (100:1 ratio) | 3 mg | Energy Boost - Vitamin/Minerals -Brain Health |
| Vitamin B2 (Riboflavin) | 65 mg | 10 mg | Energy Boost - Vitamin/Minerals |
| Vitamin B3 (Niacin) | 325 mg | 50 mg | Energy Boost - Vitamin/Minerals -Brain Health |
| Vitamin B6 | 325 mg | 50 mg | Energy Boost - Vitamin/Minerals |
| Vitamin B12 | 200 mg (100:1 ratio) | 0.2 mg | Energy Boost - Vitamin/Minerals -Brain Health |
| Vitamin C | 65 mg | 10 mg | Antioxidant- Vitamin/Minerals |
| Vitamin E | 1.95 g | 300 mg | Blood/Heart Health -Immune Support - Antioxidant-Vitamin/Minerals |
| Fish Oil | 650 mg | 100 mg | Brain Health - Antioxidant - Pain Relief -Fur/Skin/Hair Health Immune Support |
| L-Arginine | 6.5 g | 1 g | Brain Health - Blood/Heart Health |
| S-adenosyl-L-methionine | 325 mg | 50 mg | Brain Health - Pain Relief |
| Siberian Ginseng Extract | 0.65 g | 100 mg | Brain Health |
| Pomegranate Extract | 1.3 g | 200 mg | Antioxidant - Brain Health -Blood/Heart Health |
| Selenium | 100 mg (100:1 ratio) | 1 mg | Brain Health - Antioxidant |

The following is an example of a powdered formulation designed for pet joint support. The formulation can be diluted with a suitable amount of water and consumed.

TABLE 46

Formulation for Pet Joint Support

| | | | |
|---|---|---|---|
| Active Ingredient | Weight of nanoparticles (deviation from 6.5:1 Complex:API ratio) | API Dosage | Therapeutic Categories |
| Glucosamine | 650 mg | 100 mg | Antioxidant - Pain Relief |
| S-adenosyl-L-methionine | 325 mg | 50 mg | Brain Health - Pain Relief |
| MSM | 650 mg | 100 mg | Antioxidant - Pain Relief |
| Chondroitin sulfate | 325 mg | 50 mg | Antioxidant - Pain Relief |
| CBD | 500 mg (100:1 ratio) | 5 mg | Antioxidant - Antianxiety- Pain Relief |
| Fish Oil | 650 mg | 100 mg | Brain Health - Antioxidant - Pain Relief -Fur/Skin/Hair Health Immune Support |
| Krill oil | 650 mg | 100 mg | Antioxidant - Pain Relief -Blood/Heart Health |
| Hyaluronic Acid | 130 mg | 20 mg | Pain Relief - Fur/Skin/Hair Health |

The following is an example of a powdered formulation designed for a pet digestive aid. The formulation can be diluted with a suitable amount of water and consumed.

TABLE 47

Formulation for Digestive Aid Pet Water

| | | | |
|---|---|---|---|
| Active Ingredient | Weight of nanoparticles (deviation from standard 6.5:1 Complex:API ratio) | API Dosage | Therapeutic Categories |
| Ginger Root Extract | 325 mg | 50 mg | Antioxidant - Digestive Aid |
| Magnesium Oxide | 325 mg | 50 mg | Digestive Aid - Energy Boost -Blood/Heart Health - Immune Support |
| Turmeric | 487.5 mg | 75 mg | Antioxidant - Immune Support |
| Hawthorn Berry Extract | 1.3 g | 200 mg | Antioxidant - Digestive Aid-Blood/Heart Health |
| Jerusalem artichoke | 650 mg | 100 mg | Digestive Aid |
| Yucca Root | 325 mg | 50 mg | Digestive Aid - - Immune Support |
| Aluminum hydroxide | 650 mg | 100 mg | Digestive Aid -Kidney/Urinary Tract Health |
| Pumpkin seed powder | 1.3 g | 200 mg | Digestive Aid |

The following is an example of a powdered formulation designed for pet immune support. The formulation can be diluted with a suitable amount of water and consumed.

TABLE 48

Formulation for Immune Support Pet Water

| | | | |
|---|---|---|---|
| Active Ingredient | Weight of nanoparticles (deviation from 6.5:1 Complex:API ratio) | API Dosage | Therapeutic Categories |
| Vitamin A | 65 mg | 10 mg | Immune Support - Antioxidant-Vitamin/Minerals - Eye Support |
| Vitamin C | 65 mg | 10 mg | Immune Support - Antioxidant-Vitamin/Minerals |
| Vitamin E | 1.95 g | 300 mg | Blood/Heart Health -Immune Support - Antioxidant- Vitamin/Minerals |
| Pomegranate Extract | 1.3 g | 200 mg | Immune Support - Antioxidant - Brain Health - Blood/Heart Health |
| Beetroot Extract | 487.5 mg | 75 mg | Immune Support - Antioxidant -Energy Boost |
| Zinc | 32.5 mg | 5 mg | Immune Support - Energy Boost -Vitamin/Minerals |
| Echinacea | 325 mg | 50 mg | Immune Support - Energy Boost |
| Iron | 195 mg | 30 mg | Immune Support - Energy Boost -Vitamin/Minerals |
| Milk Thistle | 130 mg | 20 mg | Immune Support - Antioxidant |
| Colostrum | 3.25 g | 500 mg | Immune Support - Blood/Heart Health- Energy Boost - Weight Loss |
| Fish Oil | 650 mg | 100 mg | Immune Support - Brain Health -Antioxidant - Pain Relief -Fur/Skin/Hair Health |
| Magnesium Oxide | 325 mg | 50 mg | Immune Support - Energy Boost -Blood/Heart Health - Digestive Aid |
| Quercetin | 325 mg | 50 mg | Immune Support - Antioxidant - Brain Health |

The following is an example of a powdered formulation designed for anti-allergy pet water. The formulation can be diluted with a suitable amount of water and consumed.

TABLE 49

Formulation for Anti-Allergy Pet Water

| | | | |
|---|---|---|---|
| .Active Ingredient | Weight of nanoparticles (deviation from 6.5:1 Complex:API ratio) | API Dosage | Therapeutic Categories |
| Vitamin A | 65 mg | 10 mg | Immune Support - Antioxidant-Vitamin/Minerals - Eye Support |
| Yucca Root | 325 mg | 50 mg | Digestive Aid - - Immune Support |
| Turmeric | 487.5 mg | 75 mg | Antioxidant - Immune Support |
| Fish Oil | 650 mg | 100 mg | Immune Support - Brain Health -Antioxidant - Pain Relief -Fur/Skin/Hair Health |
| Echinacea | 325 mg | 50 mg | Immune Support - Energy Boost |
| Colostrum | 3.25 g | 500 mg | Immune Support - Blood/Heart Health-Energy Boost - Weight Loss |
| Nettle root extract | 3.25 g | 500 mg | Immune Support |
| Quercetin | 325 mg | 50 mg | Immune Support - Antioxidant - Brain Health |

The following is an example of a powdered formulation designed for pet motion water. The formulation can be diluted with a suitable amount of water and consumed.

TABLE 50

Formulation for Motion Pet Water

| | | | |
|---|---|---|---|
| Active Ingredient | Weight of nanoparticles (deviation from 6.5:1 Complex:API ratio) | API Dosage | Therapeutic Categories |
| Vitamin A | 65 mg | 10 mg | Immune Support -Antioxidant-Vitamin/Minerals -Eye Support |
| Vitamin B1 (Thiamin) | 300 mg (100:1 ratio) | 3 mg | Energy Boost -Vitamin/Minerals -Brain Health |
| Vitamin B2 (Riboflavin) | 65 mg | 10 mg | Energy Boost -Vitamin/Minerals |
| Vitamin B3 (Niacin) | 325 mg | 50 mg | Energy Boost -Vitamin/Minerals -Brain Health |
| Vitamin B6 | 325 mg | 50 mg | Energy Boost -Vitamin/Minerals |
| Vitamin B12 | 200 mg (100:1 ratio) | 0.2 mg | Energy Boost-Vitamin/Minerals -Brain Health |
| Vitamin D | 200 mg (1000:1 ratio) | 0.02 mg | Antioxidant-Vitamin/Minerals |
| Magnesium Oxide | 325 mg | 50 mg | Energy Boost-Blood/Heart Health - Digestive Aid Immune Support |
| Ashwagandha | 1.95 g | 300 mg | Energy Boost |
| Curcumin | 325 mg | 50 mg | Antioxidant -Immune Support |
| BCAAs | 6.5 g | 1 g | Energy Boost |
| Zinc | 32.5 mg | 5 mg | Energy Boost -Vitamin/Minerals -Immune Support |

The following is an example of a powdered formulation designed for pet eye support. The formulation can be diluted with a suitable amount of water and consumed.

TABLE 51

Formulation for Eye Support Pet Water

| | | | |
|---|---|---|---|
| Active Ingredient (API) | Weight of nanoparticles (deviation from 6.5:1 Complex:API ratio) | API Dosage | Therapeutic Categories |
| Vitamin A | 65 mg | 10 mg | Antioxidant- Vitamin/Minerals - Eye Support |
| Vitamin C | 65 mg | 10 mg | Immune Support - Antioxidant-Vitamin/Minerals |
| Lutein | 32.5 mg | 5 mg | Antioxidant- Eye Support |
| Zeaxanthin | 32.5 mg | 5 mg | Eye Support |
| Bilberry extract | 650 mg | 100 mg | Eye Support - Blood/Heart Health |
| Vitamin E | 1.95 g | 300 mg | Blood/Heart Health -Immune Support - Antioxidant-Vitamin/Minerals |

The following is an example of a powdered formulation designed for pet weight loss. The formulation can be diluted with a suitable amount of water and consumed.

TABLE 52

Formulation for Weight Loss Pet Water

| | | | |
|---|---|---|---|
| Active Ingredient (API) | Weight of nanoparticles (deviation from 6.5:1 Complex:API ratio) | API Dosage | Therapeutic Categories |
| Fish Oil | 650 mg | 100 mg | Immune Support - Brain Health -Antioxidant - Pain Relief -Fur/Skin/Hair Health |
| L-Carnitine | 1.3 g | 200 mg | Energy Boost - Weight Loss -Blood/Heart Health |
| L-Arginine | 6.5 g | 1 g | Brain Health - Blood/Heart Health |
| Choline | 325 mg | 50 mg | Weight Loss -Detox/Liver Health |
| CLA | 6.5 g | 1 g | Antioxidant - Weight Loss |
| Pysillium | 6.5 g | 1 g | Weight Loss - Digestive Aid -Blood/Heart Health |
| Caralluma Fibriata | 1.3 g | 200 mg | Weight Loss |
| Garcinia Cambogia | 1.3 g | 200 mg | Weight Loss |
| CBD | 500 mg (100:1 ratio) | 5 mg | Antioxidant - Antianxiety- Pain Relief |

The following is an example of a powdered formulation designed for pet weight gain. The formulation can be diluted with a suitable amount of water and consumed.

TABLE 53

Formulation for Weight Gain Pet Water

| | | | |
|---|---|---|---|
| Active Ingredient | Weight of nanoparticles (deviation from 6.5:1 Complex:API ratio) | API Dosage | Therapeutic Categories |
| Vitamin B12 | 200 mg (100:1 ratio) | 0.2 mg | Energy Boost - Vitamin/Minerals -Brain Health |
| Vitamin D | 200 mg (1000:1 ratio) | 0.02 mg | Antioxidant- Vitamin/Minerals |
| Siberian Ginseng Extract | 0.65 g | 100 mg | Brain Health |
| L-Taurine | 3.25 g | 500 mg | Energy Boost - Brain Health-Blood/Heart Health - Digestive Aid |
| Calcium Lactate | 1.3 g | 200 mg | Vitamin/Minerals |
| Zinc | 32.5 mg | 5 mg | Energy Boost - Vitamin/Minerals -Immune Support |

The following is an example of a powdered formulation designed for pregnancy pet water. The formulation can be diluted with a suitable amount of water and consumed.

TABLE 54

Formulation for Pregnancy Pet Water

| | | | |
|---|---|---|---|
| Active Ingredient | Weight of nanoparticles (deviation from 6.5:1 Complex:API ratio) | API Dosage | Therapeutic Categories |
| Iron | 195 mg | 30 mg | Immune Support - Energy Boost -Vitamin/Minerals |
| Zinc | 32.5 mg | 5 mg | Energy Boost - Vitamin/Minerals -Immune Support |
| Calcium Lactate | 1.3 g | 200 mg | Vitamin/Minerals |
| Magnesium Oxide | 325 mg | 50 mg | Energy Boost-Blood/Heart Health -Digestive Aid Immune Support |
| Vitamin A | 65 mg | 10 mg | Antioxidant- Vitamin/Minerals -Eye Support |
| Vitamin B1 (Thiamin) | 300 mg (100:1 ratio) | 3 mg | Energy Boost - Vitamin/Minerals |
| Vitamin B2 (Riboflavin) | 65 mg | 10 mg | Energy Boost - Vitamin/Minerals |
| Vitamin B3 (Niacin) | 325 mg | 50 mg | Energy Boost - Vitamin/Minerals -Brain Health |
| Vitamin B5 (Pantothenic acid) | 65 mg | 10 mg | Energy Boost - Vitamin/Minerals |
| Vitamin B6 | 325 mg | 50 mg | Energy Boost - Vitamin/Minerals |
| Vitamin B9 (Folic Acid) | 50 mg (100:1 ratio) | 0.5 mg | Energy Boost - Vitamin/Minerals -Kidney/Urinary Tract Health |
| Vitamin B12 | 200 mg (100:1 ratio) | 0.2 mg | Energy Boost - Vitamin/Minerals -Brain Health |
| Vitamin D | 200 mg (1000:1 ratio) | 0.02 mg | Antioxidant- Vitamin/Minerals |
| Vitamin E | 1.95 g | 300 mg | Blood/Heart Health -Immune Support - Antioxidant-Vitamin/Minerals |
| L-Taurine | 3.25 g | 500 mg | Energy Boost - Brain Health-Blood/Heart Health - Digestive Aid |
| Potassium | 3.25 g | 500 mg | Vitamin/Minerals |

The following is an example of a powdered formulation designed for heart support pet water. The formulation can be diluted with a suitable amount of water and consumed.

TABLE 55

Formulation for Heart Support Pet Water

| | | | |
|---|---|---|---|
| Active Ingredient | Weight of nanoparticles (deviation from 6.5:1 Complex:API ratio) | API Dosage | Therapeutic Categories |
| Bilberry extract | 650 mg | 100 mg | Eye Support - Blood/Heart Health |
| Vitamin B3 (Niacin) | 325 mg | 50 mg | Energy Boost - Vitamin/Minerals -Brain Health - Blood/Heart Health |
| Vitamin B12 | 200 mg (100:1 ratio) | 0.2 mg | Energy Boost - Vitamin/Minerals -Brain Health - Blood/Heart Health |
| Vitamin E | 1.95 g | 300 mg | Blood/Heart Health -Immune Support - Antioxidant-Vitamin/Minerals |
| Coenzyme Q10 | 500 mg (100:1 ratio) | 5 mg | Antioxidant - Brain Health -Blood/Heart Health |
| Krill oil | 650 mg | 100 mg | Antioxidant - Pain Relief -Blood/Heart Health |
| L-Carnitine | 1.3 g | 200 mg | Energy Boost - Weight Loss -Blood/Heart Health |
| L-Taurine | 3.25 g | 500 mg | Energy Boost - Brain Health-Blood/Heart Health - Digestive Aid |
| Magnesium Oxide | 325 mg | 50 mg | Energy Boost-Blood/Heart Health -Digestive Aid Immune Support |
| Naringin Extract | 650 mg | 100 mg | Blood/Heart Health |
| Pomegranate Extract | 1.3 g | 200 mg | Antioxidant - Brain Health -Blood/Heart Health |
| Hawthorn Berry Extract | 1.3 g | 200 mg | Antioxidant - Digestive Aid-Blood/Heart Health |

The following is an example of a powdered formulation designed for no-shedding support pet water. The formulation can be diluted with a suitable amount of water and consumed.

TABLE 56

Formulation for No Shedding Pet Water

| | | | |
|---|---|---|---|
| Active Ingredient | Weight of nanoparticles (deviation from 6.5:1 Complex:API ratio) | API Dosage | Therapeutic Categories |
| Fish Oil | 650 mg | 100 mg | Fur/Skin/Hair Health Immune Support - Brain Health - Antioxidant - Pain Relief |
| Zinc | 32.5 mg | 5 mg | Fur/Skin/Hair Health - Energy Boost - Vitamin/Minerals -Immune Support |
| Vitamin A | 65 mg | 10 mg | Fur/Skin/Hair Health-Vitamin/Minerals |
| Vitamin B5 (Pantothenic acid) | 65 mg | 10 mg | Fur/Skin/Hair Health -Vitamin/Minerals |
| Vitamin E | 1.95 g | 300 mg | Fur/Skin/Hair Health -Vitamin/Minerals - Antioxidant |
| Vitamin D | 200 mg (1000:1 ratio) | 0.02 mg | Fur/Skin/Hair Health -Vitamin/Minerals |

The following is an example of a powdered formulation designed for kidney/urinary tract support pet water. The formulation can be diluted with a suitable amount of water and consumed.

TABLE 57

Formulation for Kidney/Urinary Tract Support Pet Water

| | | | |
|---|---|---|---|
| Active Ingredient | Weight of nanoparticles (deviation from 6.5:1 Complex:API ratio) | API Dosage | Therapeutic Categories |
| Dong Quai Root | 6.5 g | 1 g | Kidney/Urinary Tract Health |
| Iron | 195 mg | 30 mg | Energy Boost - Vitamin/Minerals -Immune Support |
| Fish Oil | 650 mg | 100 mg | Fur/Skin/Hair Health Immune Support - Brain Health - Antioxidant -Pain Relief |
| Vitamin B1 (Thiamin) | 300 mg (100:1 ratio) | 3 mg | Energy Boost - Vitamin/Minerals |
| Vitamin B2 (Riboflavin) | 65 mg | 10 mg | Energy Boost - Vitamin/Minerals |
| Vitamin B6 | 325 mg | 50 mg | Energy Boost - Vitamin/Minerals |
| Vitamin B9 (Folic Acid) | 50 mg (100:1 ratio) | 0.5 mg | Energy Boost - Vitamin/Minerals -Kidney/Urinary Tract Health |
| Vitamin B12 | 200 mg (100:1 ratio) | 0.2 mg | Energy Boost - Vitamin/Minerals -Brain Health - Blood/Heart Health |

| | | | |
|---|---|---|---|
| Active Ingredient | Weight of nanoparticles (deviation from 6.5:1 Complex:API ratio) | API Dosage | Therapeutic Categories |
| Aluminum hydroxide | 650 mg | 100 mg | Digestive Aid -Kidney/Urinary Tract Health |
| Nettle root extract | 1.3 g | 200 mg | Kidney/Urinary Tract Health |
| Astragalus root powder | 6.5 g | 1 g | Kidney/Urinary Tract Health |
| Choline | 325 mg | 50 mg | Detox/Liver Health - Kidney/Urinary Tract Health |

The following two tables are an example of powdered formulations designed for pain and inflammation support. The formulation can be diluted with a suitable amount of water and consumed.

TABLE 58

Formulation for Pain and Inflammation Support

| | | | |
|---|---|---|---|
| Active Ingredient | Weight of nanoparticles (deviation from 6.5:1 Complex:API ratio) | API Dosage | Therapeutic Categories |
| Tree Bark | 0.244 g | 38 mg | Antioxidant - Anti-inflammatory |
| CBD | 0.195 g | 30 mg | Antioxidant - Antianxiety- Pain Relief |
| Turmeric | 0.195 g | 30 mg | Antioxidant - Immune Support |

TABLE 59

Formulation for Pain and Inflammation Support

| | | | |
|---|---|---|---|
| Active Ingredient | Weight of nanoparticles (deviation from 6.5:1 Complex:API ratio) | API Dosage | Therapeutic Categories |
| Tree Bark | 0.244 g | 38 mg | Antioxidant - Anti-inflammatory |
| CBD | 0.195 g | 30 mg | Antioxidant - Antianxiety- Pain Relief |

The following is an example of powdered formulations designed for support for depression and anxiety. The formulation can be dilutied with a suitable amount of water and consumed.

TABLE 60

Formulation for Depression and Anxiety Support

| | | | |
|---|---|---|---|
| Active Ingredient | Weight of nanoparticles (deviation from 6.5:1 Complex:API ratio) | API Dosage | Therapeutic Categories |
| Kratom | 1.3 g | 200 mg | Antianxiety- Pain Relief |
| Curcumin | 1.3 g | 200 mg | Antioxidant - Immune Support |

It will be apparent to those skilled in the art that various modifications and variations can be made without departing from the scope or spirit of this disclosure. Other embodiments will be apparent to those skilled in the art from consideration of the specification and practice disclosed herein.

What is claimed is:

1. A composition comprising a plurality of particles encapsulating a cargo entity;
wherein the particles comprise a sugar, sugar alcohol, or sugar substitute carrier that encapsulates the cargo entity;
wherein at least two particles comprise a different cargo entity;
wherein at least a first particle does not comprise the same cargo entity as at least a second particle;
wherein each cargo entity is independently selected from cannabinoids, nutraceuticals, active pharmaceutical ingredients (APIs), minerals, inorganic or organic salts or compounds, supplements, amino acids, peptides, polypeptides, plant extracts, ubiquinones, herbs, vitamins, fatty acids, and any combination thereof; and
wherein the particles have an average particle size ranging from about 200 nm to about 2,000 nm.

2. The composition of claim 1, wherein the composition is a fine powder.

3. A comestible or pharmaceutical product comprising the composition of claim 1.

4. A method for making a composition comprising a plurality of nanoparticle-encapsulated cargo entities, the method comprising:
a) recovering a first nanoparticle from a first dispersion comprising:
i) a sugar, sugar alcohol, or sugar substitute carrier that upon recovery encapsulates a first cargo entity;
ii) water; and
iii) the first cargo entity;
wherein the mass ratio of the first cargo entity to the sugar, sugar alcohol, or sugar substitute is from about 1:1,000 to about 1:3;
wherein the first cargo entity is selected from cannabinoids, nutraceuticals, active pharmaceutical ingredients (APIs), minerals, inorganic or organic salts or compounds, supplements, amino acids, peptides, polypeptides, plant extracts, ubiquinones, herbs, vitamins, fatty acids, and any combination thereof; and
b) recovering a second nanoparticle from a second dispersion comprising:
i) a sugar, sugar alcohol, or sugar substitute carrier that upon recovery encapsulates a second cargo entity;
ii) water; and
iii) the second cargo entity;
wherein the mass ratio of the second cargo entity to the sugar, sugar alcohol, or sugar substitute is from about 1:1,000 to about 1:3;
wherein the second cargo entity is selected from cannabinoids, nutraceuticals, active pharmaceutical ingredients (APIs), minerals, inorganic or organic salts or compounds, supplements, amino acids, peptides, polypeptides, plant extracts, ubiquinones, herbs, vitamins, fatty acids, and any combination thereof;
wherein the second cargo entity is different from the first cargo entity; and
c) combining the first and second nanoparticles to provide the composition comprising the plurality of nanoparticle-encasulated cargo entities.

5. The method of claim 4, further comprising grinding the composition into a fine powder.

6. The method of claim 4, wherein the first and/or second dispersion comprises less than 10% by weight of alcohol.

7. The method of claim 4, wherein recovering the first and second nanoparticles from the first and second dispersions comprises heating the first and second dispersions at a temperature equivalent to from about 210° F. to about 280° F. at a pressure of 1 atm for a sufficient time to reduce the volume of the dispersion.

8. The method of claim 7, further comprising cooling the first and second dispersions after the heating step.

9. The method of claim 7, wherein the mass ratio of the cargo entity to the sugar, sugar alcohol, or sugar substitute is from about 1:50 to 1:3.

10. The method of claim 7, wherein the mass ratio of the cargo entity to the sugar, sugar alcohol, or sugar substitute is about 1:5.

11. The method of claim 7, wherein the mass ratio of the sugar, sugar alcohol, or sugar substitute to the water is from about 1:8 to about 1:1.5 prior to the recovering step.

12. The method of claim 7, wherein the mass ratio of the sugar, sugar alcohol, or sugar substitute to the water is from about 1:6 to about 1:4 prior to the recovering step.

13. The method of claim 7, wherein the mass ratio of the sugar, sugar alcohol, or sugar substitute to the water is about 1:4 prior to the recovering step.

14. The method of claim 7, wherein the first and second dispersions are substantially free of ethanol.

15. The method of claim 7, wherein the first and second dispersions are substantially free of isopropyl alcohol.

16. The method of claim 7, wherein the first and second dispersions are free of ethanol.

17. The method of claim 7, wherein the first and second dispersions are free of isopropyl alcohol.

18. The method of claim 7, wherein the first and second dispersions are free of a carrier oil.

19. The method of claim 7, wherein the first and second dispersions consist essentially of the sugar, sugar alcohol, or sugar substitute, the water, and the cargo entity.

20. The method of claim 7, wherein the first and second dispersions consist of the sugar, sugar alcohol, or sugar substitute, the water, and the cargo entity.

21. The method of claim 7, wherein the sugar, when present, comprises allulose, glucose, dextrose, fructose, galactose, sucrose, lactose, maltose, trehalose, maltodextrin, or a combination thereof.
